SEQUENCE LISTING (33 sequences)

SEQ ID NO: 1 - protein, 193 aa, synthetic construct
MALPVTALLLPLALLLHAARPEVQLQASGGGLAQPGGSLRLSCAASGRTFSTYFMAWFRQPPGKGLEYVGGIRWSDGVPHYADSVKGRFTISRDNAKNTVYLQMNSLRAEDTAVYFCASRGIADGSDFGSYGQGTQVTVSSTTTPAPRPPTPAPTIASQPLSLRPEACRPAAGGAVHTRGLDFACSQLCCQLK

SEQ ID NO: 2 - protein, 38 aa, synthetic construct
CWLTKKKYSSSVHDPNGEYMFMRAVNTAKKSRLTDVTL

SEQ ID NO: 3 - protein, 30 aa, synthetic construct
QPFMRPVQTTQEEDGCSCRFPEEEEGGCEL

SEQ ID NO: 4 - protein, 48 aa, synthetic construct
RVKFSRSADAPAYQQGQNQLYNELNLGRREEYDVLDKRRGRDPEMGGK

SEQ ID NO: 5 - protein, 330 aa, synthetic construct
MALPVTALLLPLALLLHAARPEVQLQASGGGLAQPGGSLRLSCAASGRTFSTYFMAWFRQPPGKGLEYVGGIRWSDGVPHYADSVKGRFTISRDNAKNTVYLQMNSLRAEDTAVYFCASRGIADGSDFGSYGQGTQVTVSSTTTPAPRPPTPAPTIASQPLSLRPEACRPAAGGAVHTRGLDFACSQLCCQLKFWLPIGCAAFVVVCILGCILICWLTKKKYSSSVHDPNGEYMFMRAVNTAKKSRLTDVTLQPFMRPVQTTQEEDGCSCRFPEEEEGGCELRVKFSRSADAPAYQQGQNQLYNELNLGRREEYDVLDKRRGRDPEMGGK

SEQ ID NO: 6 - protein, 21 aa, synthetic construct
MALPVTALLLPLALLLHAARP

SEQ ID NO: 7 - protein, 44 aa, synthetic construct
TTTPAPRPPTPAPTIASQPLSLRPEACRPAAGGAVHTRGLDFAC

SEQ ID NO: 8 - protein, 120 aa, synthetic construct
EVQLQASGGGLAQPGGSLRLSCAASGRTFSTYFMAWFRQPPGKGLEYVGGIRWSDGVPHYADSVKGRFTISRDNAKNTVYLQMNSLRAEDTAVYFCASRGIADGSDFGSYGQGTQVTVSS

SEQ ID NO: 9 - protein, 8 aa, synthetic construct
SQLCCQLK

SEQ ID NO: 10 - protein, 21 aa, synthetic construct
FWLPIGCAAFVVVCILGCILI

SEQ ID NO: 11 - DNA, 993 bp, synthetic construct
  source 1..993: mol_type = other DNA
atggctctgcctgtgacagctctgctgctgcctctggctctgcttctgcatgccgctagacctgaagtgcagttgcaggcttctggcggaggacttgctcaacctggcggaagcctgagactgtcttgtgccgcctctggcagaaccttcagcacctacttcatggcctggttcagacagcctcctggcaaaggcctggaatacgttggcggaatccgttggagtgatggcgtgccacactacgccgatagcgtgaagggcagattcaccatcagccgggacaacgccaagaacaccgtgtacctccagatgaacagcctgagagccgaggataccgccgtgtacttctgtgccagcagaggaatcgccgacggcagcgattttggctcttatggccagggcacccaagtgaccgtgtccagcacaacaacccctgctcctagacctcctacaccagctcctacaatcgccagccagcctctgtctctgaggccagaggcttgtagacctgctgctggcggagccgtgcatacaagaggactggatttcgcctgcagccagctgtgctgtcagctgaagttctggctgcctatcggctgcgccgcctttgtggttgtgtgtatcctgggctgcatcctgatctgctggctgaccaagaaaaagtacagcagcagcgtgcacgaccccaacggcgagtacatgttcatgagagccgtgaacaccgccaagaagtccagactgaccgacgtgaccctccagcctttcatgaggcctgtgcagaccacacaagaagaggacggctgctcctgtcggttccccgaggaagaggaaggcggttgcgagctgagagtgaagttcagcagatccgccgacgctcctgcctatcagcagggccaaaaccagctgtacaacgagctgaacctggggagaagagaagagtacgacgtgctggacaagcggagaggcagagatcctgaaatgggcggcaaatga

SEQ ID NO: 12 - protein, 374 aa, synthetic construct
MALPVTALLLPLALLLHAARPEVQLQASGGGLAQPGGSLRLSCAASGRTFSTYFMAWFRQPPGKGLEYVGGIRWSDGVPHYADSVKGRFTISRDNAKNTVYLQMNSLRAEDTAVYFCASRGIADGSDFGSYGQGTQVTVSSTTTPAPRPPTPAPTIASQPLSLRPEACRPAAGGAVHTRGLDFACLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 13 - protein, 365 aa, synthetic construct
MALPVTALLLPLALLLHAARPEVQLQASGGGLAQPGGSLRLSCAASGRTFSTYFMAWFRQPPGKGLEYVGGIRWSDGVPHYADSVKGRFTISRDNAKNTVYLQMNSLRAEDTAVYFCASRGIADGSDFGSYGQGTQVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 14 - protein, 407 aa, synthetic construct
MALPVTALLLPLALLLHAARPEVQLQASGGGLAQPGGSLRLSCAASGRTFSTYFMAWFRQPPGKGLEYVGGIRWSDGVPHYADSVKGRFTISRDNAKNTVYLQMNSLRAEDTAVYFCASRGIADGSDFGSYGQGTQVTVSSTTTPAPRPPTPAPTIASQPLSLRPEACRPAAGGAVHTRGLDFACSQLCCQLKFWLPIGCAAFVVVCILGCILICWLTKKKYSSSVHDPNGEYMFMRAVNTAKKSRLTDVTLKRGRKKLLYIFKQPFMRPVQTTQEEDGCSCRFPEEEEGGCELRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 15 - protein, 395 aa, synthetic construct
MALPVTALLLPLALLLHAARPEVQLQASGGGLAQPGGSLRLSCAASGRTFSTYFMAWFRQPPGKGLEYVGGIRWSDGVPHYADSVKGRFTISRDNAKNTVYLQMNSLRAEDTAVYFCASRGIADGSDFGSYGQGTQVTVSSTTTPAPRPPTPAPTIASQPLSLRPEACRPAAGGAVHTRGLDFACSQLCCQLKFWLPIGCAAFVVVCILGCILICWLTKKKYSSSVHDPNGEYMFMRAVNTAKKSRLTDVTLQPFMRPVQTTQEEDGCSCRFPEEEEGGCELRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 16 - protein, 22 aa, synthetic construct
GSGATNFSLLKQAGDVEENPGP

SEQ ID NO: 17 - protein, 357 aa, synthetic construct
MLLLVTSLLLCELPHPAFLLIPRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFM

SEQ ID NO: 18 - DNA, 66 bp, synthetic construct
  source 1..66: mol_type = other DNA
ggatccggcgccaccaatttcagcctgctgaaacaggctggcgacgtggaagagaaccctggacct

SEQ ID NO: 19 - DNA, 1074 bp, synthetic construct
  source 1..1074: mol_type = other DNA
atgctgctgctggttacatctctgctgctgtgcgagctgccccatcctgcctttctgctgatccccagaaaagtgtgcaacggcatcggcatcggagagttcaaggacagcctgagcatcaacgccaccaacatcaagcacttcaagaactgcaccagcatcagcggcgacctgcacattctgcctgtggcctttagaggcgacagcttcacccacacacctccactggaccctcaagagctggacatcctgaaaaccgtgaaagagatcaccggatttctgttgatccaggcttggcccgagaaccggacagatctgcacgccttcgagaacctggaaatcatcagaggccggaccaagcagcacggccagttttctctggctgtggtgtccctgaacatcaccagcctgggcctgagaagcctgaaagaaatcagcgacggcgacgtgatcatctccggcaacaagaacctgtgctacgccaacaccatcaactggaagaagctgttcggcaccagcggccagaaaacaaagatcatcagcaaccggggcgagaacagctgcaaggctacaggccaagtgtgccacgctctgtgtagccctgaaggctgttggggacccgagcctagagattgcgtgtcctgtcggaatgtgtcccggggcagagaatgcgtggacaagtgcaatctgctggaaggcgagccccgcgagttcgtggaaaacagcgagtgcatccagtgtcaccccgagtgtctgccccaggccatgaacattacctgtaccggcagaggccccgacaactgtattcagtgcgcccactacatcgacggccctcactgcgtgaaaacatgtcctgctggcgtgatgggagagaacaacaccctcgtgtggaagtatgccgacgccggacatgtgtgccacctgtgtcaccctaattgcacctacggctgtacaggccctggcctggaaggctgtccaacaaacggacctaagatcccctctatcgccaccggcatggttggagccctgctgcttctgctggtggtggcccttggaatcggcctgttcatgtga

SEQ ID NO: 20 - DNA, 72 bp, synthetic construct
  source 1..72: mol_type = other DNA
atggctgtgatggcccctagaacactggtgctgctgctgtctggtgccctggctctgactcagacatgggcc

SEQ ID NO: 21 - DNA, 66 bp, synthetic construct
  source 1..66: mol_type = other DNA
atgctgctgctggttacatctctgctgctgtgcgagctgccccatcctgcctttctgctgatcccc

SEQ ID NO: 22 - protein, 380 aa, synthetic construct
MALPVTALLLPLALLLHAARPEVQLLESGGGLVQPGGSLRLSCAASGFTFSSYAMSWVRQAPGKGLEWVSSISGSGDYIYYADSVKGRFTISRDISKNTLYLQMNSLRAEDTAVYYCAKEGTGANSSLADYRGQGTLVTVSSFVPVFLPAKPTTTPAPRPPTPAPTIASQPLSLRPEACRPAAGGAVHTRGLDFACDIYIWAPLAGTCGVLLLSLVITLYCNHRNKRGRKKLLYIFKQPFMRPVQTTQEEDGCSCRFPEEEEGGCELRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 23 - protein, 510 aa, synthetic construct
MALPVTALLLPLALLLHAARPQVKLEESGGGLVQAGRSLRLSCAASEHTFSSHVMGWFRQAPGKERESVAVIGWRDISTSYADSVKGRFTISRDNAKKTLYLQMNSLKPEDTAVYYCAARRIDAADFDSWGQGTQVTVSSGGGGSGGGGSGGGGSGGGGSGGGGSAVQLVESGGGLVQAGDSLRLTCTASGRAFSTYFMAWFRQAPGKEREFVAGIAWSGGSTAYADSVKGRFTISRDNAKNTVYLQMNSLKSEDTAVYYCASRGIEVEEFGAWGQGTQVTVSSTSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 24 - protein, 416 aa, synthetic construct
MALPVTALLLPLALLLHAARPEVQLQASGGGLAQPGGSLRLSCAASGRTFSTYFMAWFRQPPGKGLEYVGGIRWSDGVPHYADSVKGRFTISRDNAKNTVYLQMNSLRAEDTAVYFCASRGIADGSDFGSYGQGTQVTVSSTTTPAPRPPTPAPTIASQPLSLRPEACRPAAGGAVHTRGLDFACLFPGPSKPFWVLVVVGGVLACYSLLVTVAFIIFWVRSKRSRLLHSDYMNMTPRRPGPTRKHYQPYAPPRDFAAYRSKRGRKKLLYIFKQPFMRPVQTTQEEDGCSCRFPEEEEGGCELRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 25 - protein, 404 aa, synthetic construct
MALPVTALLLPLALLLHAARPEVQLQASGGGLAQPGGSLRLSCAASGRTFSTYFMAWFRQPPGKGLEYVGGIRWSDGVPHYADSVKGRFTISRDNAKNTVYLQMNSLRAEDTAVYFCASRGIADGSDFGSYGQGTQVTVSSTTTPAPRPPTPAPTIASQPLSLRPEACRPAAGGAVHTRGLDFACLFPGPSKPFWVLVVVGGVLACYSLLVTVAFIIFWVRSKRSRLLHSDYMNMTPRRPGPTRKHYQPYAPPRDFAAYRSQPFMRPVQTTQEEDGCSCRFPEEEEGGCELRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 26 - protein, 395 aa, synthetic construct
MALPVTALLLPLALLLHAARPEVQLQASGGGLAQPGGSLRLSCAASGRTFSTYFMAWFRQPPGKGLEYVGGIRWSDGVPHYADSVKGRFTISRDNAKNTVYLQMNSLRAEDTAVYFCASRGIADGSDFGSYGQGTQVTVSSTTTPAPRPPTPAPTIASQPLSLRPEACRPAAGGAVHTRGLDFACLFPGPSKPFWVLVVVGGVLACYSLLVTVAFIIFWVRSKRSRLLHSDYMNMTPRRPGPTRKHYQPYAPPRDFAAYRSGGGSFRTPIQEEQADAHSTLARVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 27 - protein, 365 aa, synthetic construct
MALPVTALLLPLALLLHAARPEVQLQASGGGLAQPGGSLRLSCAASGRTFSTYFMAWFRQPPGKGLEYVGGIRWSDGVPHYADSVKGRFTISRDNAKNTVYLQMNSLRAEDTAVYFCASRGIADGSDFGSYGQGTQVTVSSTTTPAPRPPTPAPTIASQPLSLRPEACRPAAGGAVHTRGLDFACSQLCCQLKFWLPIGCAAFVVVCILGCILICWLTKKKYSSSVHDPNGEYMFMRAVNTAKKSRLTDVTLRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 28 - protein, 386 aa, synthetic construct
MALPVTALLLPLALLLHAARPEVQLQASGGGLAQPGGSLRLSCAASGRTFSTYFMAWFRQPPGKGLEYVGGIRWSDGVPHYADSVKGRFTISRDNAKNTVYLQMNSLRAEDTAVYFCASRGIADGSDFGSYGQGTQVTVSSTTTPAPRPPTPAPTIASQPLSLRPEACRPAAGGAVHTRGLDFACSQLCCQLKFWLPIGCAAFVVVCILGCILICWLTKKKYSSSVHDPNGEYMFMRAVNTAKKSRLTDVTLGGGSFRTPIQEEQADAHSTLARVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 29 - protein, 309 aa, synthetic construct
MALPVTALLLPLALLLHAARPEVQLQASGGGLAQPGGSLRLSCAASGRTFSTYFMAWFRQPPGKGLEYVGGIRWSDGVPHYADSVKGRFTISRDNAKNTVYLQMNSLRAEDTAVYFCASRGIADGSDFGSYGQGTQVTVSSTTTPAPRPPTPAPTIASQPLSLRPEACRPAAGGAVHTRGLDFACLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGK

SEQ ID NO: 30 - protein, 300 aa, synthetic construct
MALPVTALLLPLALLLHAARPEVQLQASGGGLAQPGGSLRLSCAASGRTFSTYFMAWFRQPPGKGLEYVGGIRWSDGVPHYADSVKGRFTISRDNAKNTVYLQMNSLRAEDTAVYFCASRGIADGSDFGSYGQGTQVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGK

SEQ ID NO: 31 - protein, 339 aa, synthetic construct
MALPVTALLLPLALLLHAARPEVQLQASGGGLAQPGGSLRLSCAASGRTFSTYFMAWFRQPPGKGLEYVGGIRWSDGVPHYADSVKGRFTISRDNAKNTVYLQMNSLRAEDTAVYFCASRGIADGSDFGSYGQGTQVTVSSTTTPAPRPPTPAPTIASQPLSLRPEACRPAAGGAVHTRGLDFACLFPGPSKPFWVLVVVGGVLACYSLLVTVAFIIFWVRSKRSRLLHSDYMNMTPRRPGPTRKHYQPYAPPRDFAAYRSQPFMRPVQTTQEEDGCSCRFPEEEEGGCELRVKFSRSADAPAYQQGQNQLYNELNLGRREEYDVLDKRRGRDPEMGGK

SEQ ID NO: 32 - protein, 330 aa, synthetic construct
MALPVTALLLPLALLLHAARPEVQLQASGGGLAQPGGSLRLSCAASGRTFSTYFMAWFRQPPGKGLEYVGGIRWSDGVPHYADSVKGRFTISRDNAKNTVYLQMNSLRAEDTAVYFCASRGIADGSDFGSYGQGTQVTVSSTTTPAPRPPTPAPTIASQPLSLRPEACRPAAGGAVHTRGLDFACLFPGPSKPFWVLVVVGGVLACYSLLVTVAFIIFWVRSKRSRLLHSDYMNMTPRRPGPTRKHYQPYAPPRDFAAYRSGGGSFRTPIQEEQADAHSTLARVKFSRSADAPAYQQGQNQLYNELNLGRREEYDVLDKRRGRDPEMGGK

SEQ ID NO: 33 - protein, 321 aa, synthetic construct
MALPVTALLLPLALLLHAARPEVQLQASGGGLAQPGGSLRLSCAASGRTFSTYFMAWFRQPPGKGLEYVGGIRWSDGVPHYADSVKGRFTISRDNAKNTVYLQMNSLRAEDTAVYFCASRGIADGSDFGSYGQGTQVTVSSTTTPAPRPPTPAPTIASQPLSLRPEACRPAAGGAVHTRGLDFACSQLCCQLKFWLPIGCAAFVVVCILGCILICWLTKKKYSSSVHDPNGEYMFMRAVNTAKKSRLTDVTLGGGSFRTPIQEEQADAHSTLARVKFSRSADAPAYQQGQNQLYNELNLGRREEYDVLDKRRGRDPEMGGK

(19) United States
(12) Patent Application Publication
(10) Pub. No.: US 2025/0195657 A1
(43) Pub. Date: Jun. 19, 2025

(54) CHIMERIC ANTIGEN RECEPTOR COMPRISING BCMA NANOBODY LINKED TO A CHIMERIC INTRACELLULAR SIGNALING DOMAIN

(71) Applicant: Neomics Pharmaceuticals LLC, Rockville, MD (US)

(72) Inventors: William A. COMRIE, Silver Spring, MD (US); Wenshan HAO, Parsippany, NJ (US)

(21) Appl. No.: 19/063,540

(22) Filed: Feb. 26, 2025

Related U.S. Application Data
(62) Division of application No. 18/347,487, filed on Jul. 5, 2023, now Pat. No. 12,263,221.
(60) Provisional application No. 63/358,399, filed on Jul. 5, 2022.

(51) Int. Cl. A61K 40/42; A61K 40/11; A61K 40/31; A61P 35/00; C07K 14/705; C07K 14/725; C07K 16/28; C12N 15/62

(52) U.S. Cl. CPC A61K 40/4215; A61K 40/11; A61K 40/31; A61P 35/00; C07K 14/7051; C07K 14/70521; C07K 16/2878; C12N 15/62; A61K 2239/13; A61K 2239/22; A61K 2239/48; C07K 2317/569; C07K 2319/33

(57) ABSTRACT

The present application relates to functionally improved third generation BCMA-CARs comprising modified intracellular co-stimulatory domains, which can be used in adoptive cell therapy, e.g., in treatment of diseases and disorders such as cancer.

### CROSS-REFERENCE TO RELATED APPLICATIONS

This application is a division of U.S. patent application Ser. No. 18/347,487, filed Jul. 5, 2023, which claims the benefit of and priority to U.S. Provisional Patent Application No. 63/358,399, filed Jul. 5, 2022, the contents of each of which are incorporated by reference herein in their entirety.

### REFERENCE TO AN ELECTRONIC SEQUENCE LISTING

The contents of the electronic sequence listing (NEOM_005_001US_SeqList_ST26.xml; Size 38,729 bytes; and Date of Creation: Jul. 5, 2023) are herein incorporated by reference in their entirety.

### TECHNICAL FIELD

The present disclosure provides a third-generation chimeric antigen receptor (CAR) that binds to B cell maturation antigen (BCMA). Such BCMA-CAR is effective in inducing T cell activation and proliferation, and provides enhanced cytotoxic effect in response to target cells.

### BACKGROUND

While adoptive cell therapies show efficacy in cancer treatment, the effectiveness of these therapies can be further improved through genetic engineering of T cells for better expansion and persistence. In developing genetically engineered T cells for such adoptive cell therapies, there is a need to introduce chimeric T cell co-stimulatory molecules that can be locally activated upon T cell engagement with pathological antigens to potently augment T cell activation and increase therapeutic efficacy. Second generation chimeric co-stimulatory molecules incorporating one co-stimulatory signaling domain from proteins of either CD28 family or TNFR family may not be optimal for induction of durable tumor remissions. Third-generation chimeric molecules combining two co-stimulatory signaling domains from CD28 family and TNER family members to further enhance T cell therapeutic potential, capitalizing on non-overlapping functions of the two families of co-stimulatory molecules, often suffer from reduced cell surface expression of the chimeric proteins combining two co-stimulatory signaling domains, preventing realization of the functional potential of the chimeric proteins.

The present application discloses a chimeric antigen receptor (CAR) that binds to B cell maturation antigen (BCMA) (BCMA-CAR) comprising a BCMA nanobody and third-generation chimeric T cell co-stimulatory molecule that incorporates a CD28 family signaling domain and a TNFR family signaling domain for enhanced T cell function. These enhanced functions include enhanced proliferation, killing of target cells and reduced adverse events associated with current leading clinical CARs. This application discloses exemplary BCMA-CAR proteins for use in the treatment of cancer, including but not limited to multiple myeloma, and methods of making and using these BCMA-CARs.

### SUMMARY

The present disclosure provides a chimeric antigen receptor that binds to a B cell maturation antigen (BCMA-CAR), comprising: (a) an extracellular domain comprising an amino acid sequence according to SEQ ID NO: 8; (b) a transmembrane domain of ICOS; and (c) a chimeric intracellular domain comprising a first, a second and at least a third signal transduction domain, wherein the first signal transduction domain comprises an ICOS intracellular domain, the second signal transduction domain comprises a truncated CD137 (4-1BB) intracellular domain, and the at least third signal transduction domain comprises a truncated CD3ζ domain.

In some embodiments, the BCMA-CAR comprises: (a) an extracellular domain comprising an amino acid sequence according to SEQ ID NO: 8; (b) a transmembrane domain of ICOS; and (c) a chimeric intracellular domain comprising a first, a second and a third signal transduction domains, wherein the first signal transduction domain comprises an ICOS intracellular domain, the second signal transduction domain comprises a truncated CD137 (4-1BB) intracellular domain, and the third signal transduction domain comprises a truncated CD3ζ domain.

The present disclosure provides a BCMA-CAR, comprising: (a) an extracellular domain comprising an amino acid sequence according to SEQ ID NO: 1; (b) a transmembrane domain; and (c) a chimeric intracellular domain comprising a first, a second and at least a third signal transduction domains, wherein the first signal transduction domain comprises an ICOS intracellular domain according to SEQ ID NO: 2, the second signal transduction domain comprises a truncated CD137 (4-1BB) intracellular domain according to SEQ ID NO: 3, and the at least third signal transduction domain comprises a truncated CD3ζ domain according to SEQ ID NO: 4.

In some embodiments, the BCMA-CAR comprises: (a) an extracellular domain comprising an amino acid sequence according to SEQ ID NO: 1; (b) a transmembrane domain; and (c) a chimeric intracellular domain comprising a first, a second and a third signal transduction domains, wherein the first signal transduction domain comprises an ICOS intracellular domain according to SEQ ID NO: 2, the second signal transduction domain comprises a truncated CD137 (4-1BB) intracellular domain according to SEQ ID NO: 3, and the third signal transduction domain comprises a truncated CD3ζ domain according to SEQ ID NO: 4.

In some embodiments, the extracellular domain further comprises a signal peptide, a hinge, or an ICOS extracellular stalk, or a combination thereof. In some embodiments, the extracellular domain further comprises a CD8a signal peptide, a CD8a hinge, an ICOS extracellular stalk, or a combination thereof. In some embodiments, the extracellular domain further comprises a CD8a signal peptide according to SEQ ID NO: 6, a CD8a hinge according to SEQ ID NO: 7, an ICOS extracellular stalk according to SEQ ID NO: 9, or a combination thereof.

In some embodiments, the extracellular domain comprises the amino acid sequence of SEQ ID NO: 1.

In some embodiments, the transmembrane domain comprises the amino acid sequence of SEQ ID NO: 10.

The present disclosure also provides a BCMA-CAR comprising the amino acid sequence of SEQ ID NO: 5. The present disclosure also provides a nucleic acid encoding the BCMA-CAR of the present disclosure. The present disclosure also provides a vector comprising the nucleic acid of the present disclosure. The present disclosure also provides a cell comprising the nucleic acid or the vector of the present disclosure. Compositions, e.g., pharmaceutical compositions, comprising the BCMA-CAR, the nucleic acid, the vector, and/or the cell of the present disclosure are also provided herein.

The present disclosure also provides a modified T cell, comprising: (a) a modification of an endogenous sequence encoding a T cell Receptor (TCR), wherein the modification reduces or eliminates a level of expression or activity of the TCR; and (b) a BCMA-CAR disclosed herein.

The present disclosure also provides a method of producing a plurality of modified T cells, wherein the method comprises: a) providing a plurality of primary T cells; b) providing a composition comprising the BCMA-CAR of the present disclosure, the nucleic acid of the present disclosure, or the vector of the present disclosure; and c) introducing into the plurality of primary T cells of (a) the composition of (b), to produce a plurality of modified T cells under conditions that stably express the BCMA-CAR within the plurality of modified T cells.

The present disclosure also provides a composition comprising any one of the BCMA-CAR of the present disclosure, the nucleic acid of the present disclosure, the vector of the present disclosure, the cell of the present disclosure, or the modified T cell of the present disclosure.

The present disclosure also provides a method of treating a disease or disorder, comprising administering to a subject in need thereof a therapeutically effective number of the cell of the present disclosure, a therapeutically effective number of the modified T cell of the present disclosure, a therapeutically effective amount of the composition of the present disclosure, or a therapeutically effective number of the plurality of modified T cells produced by the method of the present disclosure.

In some embodiments, the disease or disorder is a cancer, an autoimmune disease or disorder, or an inflammatory disease. In some embodiments, the cancer is a hematologic cancer. In some embodiments, the hematological cancer is a leukemia, a lymphoma, or a myeloma. In some embodiments, the cancer is selected from acute leukemia, acute lymphoblastic leukemia (ALL), acute lymphocytic leukemia, B cell, T cell or FAB ALL, acute myeloid leukemia (AML), acute myelogenous leukemia, chronic myelocytic leukemia (CML), chronic lymphocytic leukemia (CLL), hairy cell leukemia, myelodysplastic syndrome (MDS), Hodgkin's lymphoma, Hodgkin's disease, non-Hodgkin's lymphoma, and multiple myeloma. In some embodiments, the cancer is a multiple myeloma. In some embodiments, the cancer expresses BCMA. In some embodiments, the subject is a mammal. In some embodiments, the mammal is a human.

### BRIEF DESCRIPTION OF THE DRAWINGS

FIG. 1 shows a schematic depicting the structure and orientation of the extracellular, transmembrane, and intracellular signaling domains of the exemplary BCMA-CAR disclosed herein (“NPB5005-ICOSBBtZt” also referred to as “Receptor 1”). SP: signal peptide, BCMA: BCMA-binding nanobody, H: hinge, S: stalk, TM: transmembrane domain, ICOS: ICOS intracellular domain, 4-1BBt: 4-1BB intracellular domain with N-terminal truncation, CD3ζt: CD3ζ domain with C-terminal truncation of ITAM2 and ITAM3.

FIGS. 2A-2D show graphs depicting increased CAR surface expression by a BCMA-CAR of the present disclosure (“NPB5005-ICOSBBtZt”), as compared to several exemplary second and third generation CARs utilizing the same extracellular BCMA domain (see Table 2). FIG. 2A shows bar graphs depicting the surface expression of CAR receptors. FIG. 2B shows flow cytometry histograms of data from FIG. 2A. FIG. 2C shows BCMA mean fluorescence intensity (MFI) of the transduced T cell population. FIG. 2D shows flow cytometry histograms of data from FIG. 2C.

FIGS. 3A-3C show line graphs depicting reduced cytokine production by a BCMA-CAR of the present disclosure (“NPB5005-ICOSBBtZt”), as compared to several exemplary second and third generation CARs utilizing the same extracellular BCMA domain as controls (see Table 2). FIG. 3A shows IL-2 production. FIG. 3B shows TNFα (“TNF”) production. FIG. 3C shows IFNγ production. The x-axis depicts the ratio of effector CAR T cells to target tumor cells (E:T). The y-axis depicts the cytokine production as measured by multiplex assay on 18-hr co-culture supernatants.

FIGS. 4A-4C show line graphs depicting the killing of tumor cell lines by a BCMA-CAR of the present disclosure (“NPB5005-ICOSBBtZt”) during co-culture compared to effector CAR T cell therapy controls targeting BCMA (bb2121, FHVH33, and LCARB38M). FIG. 4A shows percent killing of MM.1S tumor cells. FIG. 4B shows the percent killing of U266 tumor cells. FIG. 4C shows percent killing of RPMI8226 tumor cells. The x-axis depicts the ratio of effector CAR T cells to target tumor cells (E:T). The y-axis depicts the percentage of tumor cells killed. The effector CAR T cells are as indicated.

FIGS. 5A-5C depict effects of multiple stimulations by a BCMA-CAR of the present disclosure (“NPB5005-ICOSBBtZt”). FIG. 5A shows a schematic depiction of a method of testing proliferation and function of T cells expressing an exemplary BCMA-CAR disclosed herein (“NPB5005-ICOSBBtZt”) compared to T cells expressing control CARs. RPMI8226 myeloma cells expressing BCMA were co-cultured with BCMA-CAR T cells for a total of 16 days and stimulated with 100,000 additional fresh RPMI8226 cells on Day 4, Day 8, and Day 12. FIG. 5B shows line graphs depicting cumulative T cell number over multiple stimulations. FIG. 5C shows line graphs depicting target cell number at the end of each stimulation.

FIGS. 6A-6C shows a series of graphs depicting surface expression of PD-1 on BCMA-CAR T cells of the present disclosure (“NPB5005-ICOSBBtZt”) compared to control CAR T cells following the third stimulation with target cells in the exemplary model depicted in FIG. 5. FIG. 6A are PD1 flow cytometry plots with T cells from two separate donors (top and bottom panels). FIG. 6B shows bar graphs depicting percentage cells expressing PD1 for RPMI-8226 cells. FIG. 6C shows bar graphs depicting percentage cells expressing PD1 for U266 cells.

FIGS. 7A-7B are line graphs depicting functionality of a BCMA-CAR of the present disclosure (“NPB5005-ICOSBBtZt”). FIG. 7A depicts line graphs of IL2 production after an 18-hour co-culture of CAR-transduced cells with RPMI-8226 cells at the indicated effector to target (E:T) ratio. FIG. 7B depicts line graphs of CAR positive cells over multiple stimulations with RPMI-8226 multiple myeloma cells.

FIGS. 8A-8D depict the proliferative and cytolytic properties of a BCMA-CAR of the present disclosure (“NPB5005-ICOSBBtZt”) are maintained in clinical vector format, which lacks a eukaryotic selection marker. FIG. 8A shows flow cytometry plots of T cells transduced with NPB5005-ICOSBBtZt and reference CAR constructs and K562-BCMA engineered target cells over multiple repeated stimulations. FIG. 8B are plots depicting K562.GFP (left) and K562.BCMA.GFP (right) cell number after a 96-hour co-culture with T cells transduced with the indicated CAR constructs. FIG. 8C are plots depicting T cell number after a 96-hour co-culture with either K562.GFP (left) or K562.BCMA.GFP (right) cells. FIG. 8D depicts bar graphs of target (left) and T cell numbers (right) of the indicated CAR-T cell population and K562.BCMA.GFP target cell population after multiple stimulations.

FIGS. 9A-9B depict the cytokine production and PD1 expression properties of a BCMA-CAR of the present disclosure (“NPB5005-ICOSBBtZt”) are maintained in clinical vector format, which lacks a eukaryotic selection marker. NPB5005-ICOSBBtZt maintains lower cytokine production and PD1 expression. FIG. 9A shows line graphs evaluating cytokine production by CAR-transduced T cells at various E:T ratios in response to stimulation with K562.GFP and K562.BCMA.GFP target cells. FIG. 9B shows plots of PD1 expression on the surface of T cells transduced with the indicated CAR constructs in response to K562.BCMA.GFP target cells.

FIGS. 10A-10C depict intermediate CAR-intrinsic tonic signaling of a BCMA-CAR of the present disclosure (“NPB5005-ICOSBBtZt”). FIG. 10A shows flow cytometry plots depicting T cell proliferation marked by dilution of Tag-It Violet dye of T cells after 96 hours treated with the indicated stimulation condition. FIG. 10B depicts bar graphs quantifying the cell numbers shown in FIG. 10A, with a zoomed-in panel showing T cell number in the absence of exogenous stimulation. FIG. 10C shows bar graphs depicting cytokine production at the indicated E:T ratio in the absence of the BCMA target protein.

FIGS. 11A-11D depict dose response of a BCMA-CAR of the present disclosure in a murine model in vivo. FIG. 11A shows intravital imaging of MM.1S xenograft tumor burden in mice after being treated with increasing doses of NPB5005-ICOSBBtZt at post-infusion days 4, 10, and 18. FIG. 11B shows measurement of tumor burden (using soluble luciferase) from MM.1S tumor-bearing mice treated with the indicated T cell population. FIG. 11C shows the survival curve for MM.1S tumor-bearing female mice treated with the indicated T cell population. FIG. 11D shows the survival curve for MM.1S tumor-bearing male mice treated with the indicated T cell population.

In certain Figures and Examples described herein, the following abbreviations are used to describe domains of BCMA-CARs: “CD28” or “28” for CD28; “2” for CD2; “3” for CD3; “4” for CD4; “CD8” for CD8a; “BB” for 4-1BB; “BBt” for truncated 4-1BB; “Z” for CD3ζ; “Zt” for truncated CD3ζ; “OX40” or “40” for OX-40, and “40t” or “OX40t” for truncated OX-40. Domains are ordered from BCMA-binding domain to extracellular domain(s) to intracellular domain(s).

### DETAILED DESCRIPTION

Provided herein are improved chimeric antigen receptors that bind to B cell maturation antigen (BCMA-CAR) comprising: (a) an extracellular domain; (b) a transmembrane domain; and (c) a chimeric intracellular domain comprising a first, a second and at least a third signal transduction domain, wherein the first signaling domain is based on the intracellular signaling domain of a CD28 family protein (e.g., ICOS), the second signaling domain comprises a mutant intracellular signaling domain of a tumor necrosis factor receptor (TNFR) family protein (e.g., 4-1BB), and the at least third intracellular signaling domain is derived from a CD3 signaling domain (e.g., CD3ζ).

Provided herein is a BCMA-CAR (e.g., NPB5005-ICOSBBtZt) that displays high on-target efficacy and low levels of adverse phenotypic events. The BCMA-CAR of the present disclosure (e.g., NPB5005-ICOSBBtZt) provides increased effector cell (e.g., T cell) function, proliferation, and retains such phenotypes over multiple stimulations. Further, the BCMA-CAR of the present disclosure (e.g., NPB5005-ICOSBBtZt) produces low cytokine levels and low surface PD-1 expression. Such properties of the BCMA-CAR of the disclosure (e.g., NPB5005-ICOSBBtZt) provide numerous technical advantages over control BCMA-CARs tested.

In some embodiments, a BCMA-CAR of the present disclosure (e.g., NPB5005-ICOSBBtZt) retains increased effector cell function (e.g., T cell proliferation) over multiple rounds of stimulation, e.g., two rounds, three rounds, four rounds, with target cells, e.g., cancer cells. In certain embodiments, the increase in effector cell function (e.g., T cell proliferation) is at least two-fold, at least three-fold, at least four-fold, or at least five-fold over a control BCMA-CAR. In some embodiments, a BCMA-CAR of the present disclosure (e.g., NPB5005-ICOSBBtZt) retains increased effector cell function (e.g., target cell cytolysis, e.g., cancer cell cytolysis) over multiple rounds of stimulation, e.g., two rounds, three rounds, four rounds, with target cells, e.g., cancer cells. In certain embodiments, the increase in effector cell function (e.g., target cell cytolysis, e.g., cancer cell cytolysis) is at least two-fold, at least three-fold, at least four-fold, or at least five-fold over a control BCMA-CAR.

In some embodiments, a BCMA-CAR of the present disclosure (e.g., NPB5005-ICOSBBtZt) retains decreased adverse phenotypic events (e.g., surface PD-1 expression) over multiple rounds of stimulation, e.g., two rounds, three rounds, four rounds, with target cells, e.g., cancer cells. In certain embodiments, the decreased adverse phenotypic events (e.g., surface PD-1 expression) is at least two-fold, at least three-fold, at least four-fold, or at least five-fold over a control BCMA-CAR. In some embodiments, a BCMA-CAR of the present disclosure (e.g., NPB5005-ICOSBBtZt) retains decreased adverse phenotypic events (e.g., tonic cytokine production, e.g., IL-2, IFNγ, or TNFα) over multiple rounds of stimulation, e.g., two rounds, three rounds, four rounds, with target cells, e.g., cancer cells. In certain embodiments, the decreased adverse phenotypic events (e.g., tonic cytokine production, e.g., IL-2, IFNγ, or TNFα) is at least two-fold, at least three-fold, at least four-fold, or at least five-fold over a control BCMA-CAR.

In some aspects, the chimeric intracellular domain of the BCMA-CAR of the present disclosure enhances the activity and efficacy of the BCMA-CAR. For example, when expressed in T cells, the BCMA-CAR of the present disclosure enhances T cell stimulation, proliferation, persistence, and killing of target myeloma cells expressing BCMA. BCMA-CAR T cells can eliminate BCMA-positive multiple myeloma cells with equivalent potency and superior efficacy to the leading clinical CAR T cell therapies directed against BCMA (e.g., bb2121, FHVH33, and LCARB38M) while producing significantly less cytokines. Accordingly, in some embodiments, the combination of improved T cell proliferation, persistence, tumor cell killing, reduced inflammatory cytokine production, and lower surface expression of PD-1, make the BCMA-CAR proteins of the present disclosure superior to leading BCMA-CAR T cell therapies.

Tumor associated antigens and tumor specific antigens allow for the immunological targeting of the tumor with relatively minimal risk of off-tumor, on-target side effects. Tumor cells can upregulate these antigens which can then be targeted by the human immune response. The disclosure herein provides a chimeric intracellular domain that combines an ICOS intracellular domain and a 4-1BB intracellular domain to provide a co-stimulatory molecule that exhibits superior functionality to other CD28-or 4-1BB-based receptors to generate a BCMA-CAR product that contributes to enhanced T cell proliferation, killing efficacy and resistance to the suppressive function of the tumor microenvironment.

In some aspects, the present disclosure also provides a nucleic acid encoding the BCMA-CAR disclosed herein. In some embodiments, the nucleic acid encoding the BCMA-CAR disclosed herein comprises a nucleotide sequence according to SEQ ID NO: 11.

In some embodiments, the nucleic acid disclosed herein comprises a nucleic acid sequence encoding a chimeric intracellular domain. In some embodiments, the BCMA-CAR disclosed herein is for expression in a T cell, wherein the T cell co-expresses at least one of the endogenous co-stimulatory molecules CD28, CD2, OX-40, ICOS, CD28, CD3, CD4, CD8 and CD40L or a combination thereof.

In some aspects, the present disclosure also provides a vector comprising the nucleic acid disclosed herein. In some embodiments, the vector disclosed herein is any one of a viral vector, a plasmid, a mini-circle DNA, a cosmid, a yeast artificial chromosome, a bacterial artificial chromosome, or a transposon/transposase system. In some embodiments, the viral vector is an adeno-viral vector or a lentiviral vector. In some embodiments, the vector is a lentiviral vector.

In some aspects, the present disclosure also provides a cell comprising the nucleic acid or the vector disclosed herein. In some embodiments, the cell disclosed herein is a modified T cell. In some embodiments, the modified T cell is an allogeneic T cell. In some embodiments, the modified T cell is an autologous T cell. In some embodiments, the modified T cell is any one of a naïve T cell, an early memory T cell, a stem cell-like T cell, a stem memory T cell (TSCM), a central memory T cell (TCM) and a regulatory T cell (Treg).

In some embodiments, the BCMA-CAR comprises an extracellular domain of SEQ ID NO: 1. In some embodiments, the BCMA-CAR comprises a first signal transduction domain of SEQ ID NO: 2, a second signal transduction domain of SEQ ID NO: 3, and a third signal transduction domain of SEQ ID NO: 4. In some embodiments, the BCMA-CAR comprises the sequence of SEQ ID NO: 5.

### Definitions

Unless otherwise indicated, “B cell maturation antigen”, “recombinant B cell maturation antigen”, and “BCMA” are used interchangeably. B cell maturation antigen, also known as CD269, is a member of the tumor necrosis factor receptor superfamily 17 is highly selectively expressed on the surface of multiple myeloma (MM) cells (Guo, R et al. Front Immunol vol. 13:839097). Under physiological conditions, BCMA is mainly expressed on plasmablasts and terminally differentiated plasma cells (PCs). In the pathological case, BCMA is expressed nearly on all MM tumor cell lines (80%-100%), and the quantity of BCMA on the surface of malignant PCs is much higher than regulator PCs (Lee L, et al. Br J Haematol (2016) 174:911-22). Its expression is restricted to the B-cell lineage and has been shown to be important for B cell development and autoimmune response.

As used herein, “BCMA specific” (e.g., a BCMA specific T cell receptor or BCMA specific chimeric antigen receptor) refers to a binding agent that binds selectively to an antigen or epitope of BCMA, such as with a high affinity, and does not significantly bind to other unrelated antigens or epitopes.

“CD137” as described herein is a member of the tumor necrosis factor (TNF) receptor family, and also referred to as 4-1BB, CD137, tumor necrosis factor receptor superfamily member 9 (TNFRSF9) and induced by lymphocyte activation (ILA). As described herein, the terms “CD137”, “4-1BB”, “4-1BB wt”, “4-1BB wild type”, “BB”, “BB wt” and “BB wild type” are used interchangeably throughout, for example, when describing constructs or co-stimulatory molecules of the present application, unless otherwise indicated.

A truncated CD137 intracellular domain as described herein, is referred to as “truncated CD137”, “CD137t”, “truncated 4-1BB”, “4-1BBt”, “truncated BB” or “BBt” interchangeably throughout, for example, when describing constructs or co-stimulatory molecules of the present application, unless otherwise indicated. In some embodiments, the mutant CD137 intracellular domain comprises a deletion of one, two, three or four lysine residue(s) from amino acid position 1 to amino acid position 12 of the N-terminus of the CD137 intracellular domain, of the present disclosure. In some embodiments, the mutant CD137 intracellular domain comprises one or more lysine mutation(s) from amino acid position 1 to amino acid position 12 of the N-terminus of the CD137 intracellular domain, of the present disclosure. In some embodiments, the mutant CD137 intracellular domain comprises one or more lysine mutation(s) at amino acid positions selected from amino acid positions 1, 5, 6 and 12 of the N-terminus of the CD137 intracellular domain, of the present disclosure. In some embodiments, the one or more lysine mutation(s) are lysine to alanine mutations. In some embodiments, the CD137 intracellular domain comprises an amino acid sequence according to SEQ ID NO: 3.

Unless indicated otherwise, the terms “co-stimulatory molecule”, “costimulatory molecule”, “co stimulatory molecule”, “co-stimulatory protein”, “costimulatory protein”, “co stimulatory protein”, “co-stimulatory receptor”, “costimulatory receptor” “co stimulatory receptor” and “switch receptor” are used interchangeably, to refer to the recombinant T cell co-stimulatory receptors (RTCRs) comprising the novel chimeric co-stimulatory intracellular domains of the present application. These terms may be used in combination with terms such as “recombinant T cell”, “recombinant”, “chimeric T cell”, and “chimeric”, to refer to the RTCRs of the present application.

As described herein, “a recombinant T cell co-stimulatory receptor” or “switch receptor” of the present disclosure is a “costimulatory molecule” “co-stimulatory receptor” or “co-stimulatory protein” generated by operably linking an extracellular domain to an intracellular chimeric intracellular protein of the present disclosure.

The terms T cell, T-cell, t cell, t-cell, and T lymphocyte can be used interchangeably in the present disclosure.

The terms “NPB5005-ICOSBBtZt”, “BCMA-ICOSBBtZt”, “NPB5005-BCMA-ICOSBBtZt”, “Receptor 1”, “receptor 1”, “Construct 1”, “construct 1”, and “V283” are used interchangeably in the present disclosure to refer to an exemplary BCMA CAR of the present disclosure or T cell expressing an exemplary BCMA CAR of the present disclosure.

The terms “subject” and “patient” refer to an organism to be treated by the methods and compositions described herein.

The term “about” or “approximately” can be understood as within 10%, 9%, 8%, 7%, 6%, 5%, 4%, 3%, 2%, 1%, 0.5%, 0.1%, 0.05%, or 0.01% of the stated value. In some embodiments, “about” or “approximately” can be understood as within 5%, 4%, 3%, 2%, 1%, 0.5%, 0.1%, 0.05%, or 0.01% of the stated value. In some embodiments, “about” or “approximately” can be understood as within 2%, 1%, 0.5%, 0.1%, 0.05%, or 0.01% of the stated value. In some embodiments, “about” or “approximately” can be understood as within 1%, 0.5%, 0.1%, 0.05%, or 0.01% of the stated value.

Throughout the disclosure, where compositions are described as having, including, or comprising specific components, or where processes and methods are described as having, including, or comprising specific steps, it is contemplated that, additionally, there are compositions of the present disclosure that consist essentially of, or consist of, the recited components, and that there are processes and methods according to the present disclosure that consist essentially of, or consist of, the recited processing steps.

### Extracellular Domain

Provided herein is a BCMA-CAR comprising an extracellular domain.

In some embodiments, the extracellular domain comprises a protein, a peptide, a glycoprotein, an antibody or a fragment thereof that binds to BCMA, e.g., an antigen-binding fragment. In some embodiments, the antibody or fragment thereof is a Fab fragment, a F(ab)₂ fragment, a diabody, a nanobody, a sdAb, Fv, a V_{H}H fragment, or a single chain Fv fragment. In some embodiments, the antibody or fragment thereof is a nanobody. In some embodiments, the nanobody comprises the amino acid sequence having at least 50%, 55%, 60%, 65%, 70%, 75%, 80%, 85%, 90%, 95%, 97% or 99% identity to the amino acid sequence of SEQ ID NO: 8. In some embodiments, the nanobody comprises the amino acid sequence of SEQ ID NO: 8.

In some embodiments, the extracellular domain is a B cell maturation antigen (BCMA) binding protein. In some embodiments, the BCMA binding protein is a BCMA specific T cell receptor (TCR). In some embodiments, the BCMA binding protein is a nanobody. In some embodiments, the BCMA binding protein is a BCMA specific chimeric antigen receptor (CAR).

In some embodiments, the extracellular domain comprises two or more binding sites for targeting two or more non-identical target antigens. In some embodiments, the extracellular domain comprises two or more binding sites for targeting two or more non-identical sites on a target antigen. In some embodiments, the extracellular domain comprises two antigen binding domains or fragments of a bispecific antibody. In some embodiments, the extracellular domain comprises a F(ab)₂ fragment of a bispecific antibody. In some embodiments, the extracellular domain comprises two or more antigen binding domains or fragments of a multi-specific antibody.

### Signal Peptide

In some embodiments, the extracellular domain comprises a signal peptide, e.g., at the N-terminus. In some embodiments, the signal peptide can be derived from a surface expressing protein or a secretory protein. In some embodiments, the signal peptide can be derived from Preprolactin, HIV pre-Env, HCV polyprotein, CB virus polyprotein, Pestivirus polyprotein, Precalreticulin, pre-VSV-G, HLA class I histocompatibility antigen or PD-1 signal peptide (PD-1 SP), interleukin 12 (IL12), GM-CSF, or CD8 alpha chain (CD8a) signal peptide.

In some embodiments, the signal peptide is derived from a CD8a signal peptide. In some embodiments, the CD8a signal peptide comprises an amino acid sequence having at least 50%, 55%, 60%, 65%, 70%, 75%, 80%, 85%, 90%, 95%, 97% or 99% identity to the amino acid sequence of SEQ ID NO: 6. In some embodiments, the CD8a signal peptide comprises an amino acid sequence of SEQ ID NO: 6.

### Hinge

In some embodiments, the extracellular domain comprises a hinge region. In some embodiments, the hinge region is derived from CD8, PD-1, CD28, ICOS, or IgG hinge domain. In some embodiments, the hinge region is derived from CD8 alpha chain (CD8a) hinge. In some embodiments, the CD8a hinge region comprises an amino acid sequence having at least 50%, 55%, 60%, 65%, 70%, 75%, 80%, 85%, 90%, 95%, 97% or 99% identity to the amino acid sequence of SEQ ID NO: 7. In some embodiments, the CD8a hinge region comprises the amino acid sequence of SEQ ID NO: 7.

### Stalk

In some embodiments, the extracellular domain comprises a stalk region. In some embodiments, the stalk region is derived from CD8, PD-1, CD28, ICOS, or IgG. In some embodiments, the hinge region is derived from ICOS. In some embodiments, the ICOS stalk region comprises an amino acid sequence having at least 50%, 55%, 60%, 65%, 70%, 75%, 80%, 85%, 90%, 95%, 97% or 99% identity to the amino acid sequence of SEQ ID NO: 9. In some embodiments, the ICOS stalk region comprises the amino acid sequence of SEQ ID NO: 9.

In some embodiments, the extracellular domain further comprises one or more of a signal peptide, a hinge, and an ICOS extracellular stalk, or a combination thereof. In some embodiments, the extracellular domain further comprises one or more of a CD8a signal peptide, a CD8a hinge, an ICOS extracellular stalk, or a combination thereof.

In some embodiments, the CD8a signal peptide comprises an amino acid sequence according to SEQ ID NO: 6. In some embodiments, the CD8a hinge comprises an amino acid sequence according to SEQ ID NO: 7. In some embodiments, the ICOS extracellular stalk comprises an amino acid sequence according to SEQ ID NO: 9. In some embodiments, the extracellular domain further comprises one or more of a CD8a signal peptide according to SEQ ID NO: 6, a CD8a hinge according to SEQ ID NO: 7, and an ICOS extracellular stalk according to SEQ ID NO: 9, or a combination thereof.

In some embodiments, the extracellular domain comprises the amino acid sequence of SEQ ID NO: 1.

### Transmembrane Domain

In some embodiments, the transmembrane domain of the BCMA-CAR disclosed herein is derived from CD8, PD1, CD28, ICOS, or IgG transmembrane domain. In some embodiments, the transmembrane domain is located between the extracellular domain and the first signal transduction domain. In some embodiments, the transmembrane domain is derived from an ICOS transmembrane domain. In some embodiments, the ICOS transmembrane domain comprises an amino acid sequence having at least 50%, 55%, 60%, 65%, 70%, 75%, 80%, 85%, 90%, 95%, 97% or 99% identity to the amino acid sequence of SEQ ID NO: 10. In some embodiments, the ICOS transmembrane domain comprises the amino acid sequence of SEQ ID NO: 10.

### Intracellular Signaling Domains

### ICOS Domain

In some embodiments, the BCMA-CAR of the present disclosure comprises a first signal transduction domain derived from an ICOS protein.

The CD28 family proteins have a single extracellular immunoglobulin variable-like (IgV) domain followed by a short cytoplasmic tail. Members of the CD28 family proteins include CD28, CD28H, inducible costimulator (ICOS), cytotoxic T lymphocyte-associated antigen 4 (CTLA-4, CD152), program death-1 (PD-1), and B-and T-lymphocyte attenuator (BTLA). CD28, CD28H, and ICOS are co-stimulatory proteins that are expressed on T cells and, for example, promote their activation, high levels of cytokine/chemokine expression, resistance to apoptosis, and proliferation of T cells. In some embodiments, the first signaling domain that is based on the intracellular signaling domain of a CD28 family protein is an ICOS protein.

The “ICOS protein” as described herein is an inducible T cell co-stimulatory protein, also referred to as AILIM, CD278, CCLP, CRP-1, H4, Ly115 and CVID1. In some embodiments, the ICOS intracellular domain can be from a mammalian ICOS. In some embodiments, the mammalian ICOS can be a human ICOS, a mouse ICOS, a rat ICOS or a monkey ICOS. In some embodiments, the ICOS intracellular domain can be derived from a human ICOS, or an isoform or a variant thereof, e.g., comprising an amino acid sequence identical to any one of the human ICOS amino acid sequences according to GenBank Accession Nos: AAH28006.1, NP_036224.1, AIC51287.1, AIC60036.1, NP_036224.1, Q9Y6W8.1, EAW70357.1, EAW70356.1, EAW70355.1, AAL40934.1, AAL40933.1, CAC06612.1, AAX93073.1, AAM00909.1, AAH28210.1 and CAD59742.1. In some embodiments, the ICOS intracellular domain can be derived from a mouse ICOS, or an isoform or a variant thereof, e.g., comprising an amino acid sequence identical to any one of the mouse ICOS amino acid sequences according to GenBank Accession Nos: NP_059508.2, Q9WVS0.2, EDL00161.1, CAM13242.1, CAM13241.1, CAB71153.1, AAG48732.1, AAH34852.1, XP_006496203.1, XP_006496202.1, XP_006496201.1, ACX50464.1, ACX50463.1, AAH28006.1, XP_021052880.1, XP_029334968.1 and XP_021030282.1. In some embodiments, the ICOS intracellular domain can be derived from a rat ICOS, or an isoform or a variant thereof, e.g., comprising an amino acid sequence identical to any one of the rat ICOS amino acid sequences according to GenBank Accession Nos: NP_072132.1, Q9R1T7.1, XP_008765358.1, XP_006245100.1, XP_006245099.1, EDL98922.1, EDL98921.1, XP 038940099.1, XP 032755449.1, XP_017457364.1, XP_006256324.1, XP 006256323.1, XP 006256322.1, XP_029425757.1, XP_029425757.1, XP_021119236.1, XP 012929934.1, XP 012867370.1 and XP_012867363.1. In some embodiments, the ICOS intracellular domain can be derived from a non-human primate ICOS, or an isoform or a variant thereof, e.g., comprising an amino acid sequence identical to any one of the monkey ICOS amino acid sequences according to GenBank Accession Nos: XP_007964137.1, NP_001253918.1, XP_010350939.1, XP_012301785.1, XP_012301784.1, XP_017739861.1, XP 010334714.1, XP 003925677.1, AFH29328.1, XP_008997520.1, XP_023075107.1, XP 023075099.1, XP 021779593.1, XP_003907887.1, XP_025260988.1, XP_025260987.1, XP 025260986.1, XP 011716287.1, XP_011716285.1, XP_005574075.1, XP_011903009.1, XP 011805288.1, XP 011805287.1, XP 011847867.1, XP_011847866.1, XP_017392362.1, XP 033086489.1, XP 032134414.1, XP_032134413.1, and XP_017802331.1.

In some embodiments, the chimeric intracellular domain comprises a first signal transduction domain derived from a protein of the CD28 family. In some embodiments, the first signal transduction domain is derived from any one of CD28, CD28H, ICOS, or a combination thereof.

In some embodiments, the first signal transduction domain derived from ICOS comprises an amino acid sequence according to SEQ ID NO: 2. In some embodiments, the first signal transduction domain derived from ICOS comprises an amino acid sequence comprising having at least 50%, 55%, 60%, 65%, 70%, 75%, 80%, 85%, 90%, 95%, 97% or 99% identity to the amino acid sequence of SEQ ID NO: 2.

### 4-1BB Intracellular Domain

The Tumor Necrosis Factor Receptor (TNFR) family proteins includes TNFRI (tumor necrosis factor receptor 1/TNFRSFIA), TNFR2 (tumor necrosis factor receptor 2/TNFRSF1B), lymphotoxin β receptor/TNFRSF3, OX40/TNFRSF4, CD40/TNFRSF5, Fas/TNFRSF6, decoy receptor 3/TNFRSF6B, CD27/TNFRSF7, CD30/TNFRSF8, 4-1BB/TNFRSF9, DR4 (death receptor 4/TNFRSF10A), DR5 (death receptor 5/TNFRSF10B), decoy receptor 1/TNFRSF10C, decoy receptor 2/TNFRSF10D, RANK (receptor activator of NF-kappa B/TNFRSF11A), OPG (osteoprotegerin/TNFRSF11B), DR3 (death receptor 3/TNFRSF25), TWEAK receptor/TNFRSF12A, TACI/TNFRSF13B, BAFF-R (BAFF receptor/TNFRSF13C), HVEM (herpes virus entry mediator/TNFRSF14), nerve growth factor receptor/TNFRSF16, BCMA (B cell maturation antigen/TNFRSF17, GITR (glucocorticoid-induced TNF receptor/TNFRSF18), TAJ (toxicity and JNK inducer/TNFRSF19), RELT/TNFRSF19L, DR6 (death receptor 6/TNFRSF21), TNFRSF22, TNFRSF23, ectodysplasin A2 isoform receptor/TNFRS27 and ectodysplasin 1-anhidrotic receptor. Interactions between tumor necrosis factor superfamily (TNFSF) ligands and TNF receptor superfamily (TNFRSF) receptors provide the co-stimulatory signals that control the survival, proliferation, differentiation, and effector function of immune cells. Depending upon the specific intracellular signal induced by TNFRSF members, they can be categorized into three groups—death domain (DD)-containing receptors, decoy receptors, and TNF receptor-associated factor (TRAF)-binding receptors. Some TNFRSFs such as TNFR-1, Fas, DR3, DR4, DR5, and DR6, contain their own DDs and/or interact with other cytoplasmic DD-containing adaptor molecules. Some other TNFRSFs, such as TNFR-2, CD27, CD30, CD40, glucocorticoid-induced TNFR family-related gene (GITR), Fn1, lymphotoxin beta-receptor (LTβR), OX40, receptor activator of NF-κB (RANK), and XEDAR, lack a DD and contain motifs with four to six amino acids called TRAF-interacting motifs (TIMs) which recruits TRAF proteins. TRAF proteins are adaptor molecules that activate multiple downstream signaling pathways such as NF-κB, Janus kinase (JNK), ERK, p38MAPK, and PI3K that help in cell survival, proliferation, and cytokine production. In some embodiments, the second signaling domain is based on a mutant of the intracellular signaling domain of a TNFR family protein and is CD137 (4-1BB).

In some embodiments, the CD137 intracellular domain can be derived from a mammalian CD137. In some embodiments, the mammalian CD137 can be derived from a human CD137, a mouse CD137, a rat CD137, or a non-human primate CD137. In some embodiments, the CD137 intracellular domain can be derived from a human CD137, or an isoform or a variant thereof, e.g., comprising an amino acid sequence identical to any one of the human CD137 amino acid sequences according to GenBank Accession Nos: U03397, AAA62478, NP_001552, Q07011, AAH06196 and XP_006710681. In some embodiments, the CD137 intracellular domain can be derived from a mouse CD137, or an isoform or a variant thereof, e.g., comprising an amino acid sequence identical to any one of the mouse CD137 amino acid sequences according to GenBank Accession Nos: NP_001070977.1, NP_001070976.1, NP_035742.1, NP_033430.1, P20334.1, XP_011248530.1, XP 011248530.1, ABI30213.1, BAE32724.1 and AAH28507.1. In some embodiments, the CD137 intracellular domain can be derived from a rat CD137, or an isoform or a variant thereof, e.g., comprising an amino acid sequence identical to any one of the rat CD137 amino acid sequences according to GenBank Accession Nos: NP_852049.1, NP_001020944.1, BAD99404.1, XP_008762504.1, XP_006239534.1, EDL81196.1, AAH97483.1, EHB16663.1, EHB16663.1, KFO38282.1, XP_010618177.1, XP_029414155.1, XP 029414154.1, XP 021099219.1 and XP_012888584.1. In some embodiments, the CD137 intracellular domain can be from a non-human primate CD137, or an isoform or a variant thereof, comprising an amino acid sequence identical to any one of the non-human primates CD137 amino acid sequences according to GenBank Accession Nos: ABY47575.1, ABI30212.1, ABY47577.1, ABY47576.1 and ABY47578.1.

In some embodiments, the CD137 intracellular domain, as described herein, comprises an amino acid sequence starting from the amino acid position 214 to the last amino acid at the C-terminal end of the amino acid sequence of the human CD137 protein, as described herein.

In some embodiments, the CD137 intracellular domain, as described herein, comprises an amino acid sequence starting from the amino acid position 215 to the last amino acid at the C-terminal end of the amino acid sequence of the mouse CD137 protein, as described herein.

In some embodiments, the mutant CD137 intracellular domain described herein is derived from any one of the CD137 proteins as described herein, comprising one or more mutation(s), wherein the mutation can be an addition/insertion, deletion/truncation, or substitution/replacement of one or more amino acids within the amino acid sequence of the CD137 protein. In some embodiments, the mutant CD137 intracellular domain described herein is any one of the CD137 intracellular domain sequences, as described herein, comprising one or more mutation(s), wherein the mutation can be an addition/insertion, deletion/truncation, or substitution/replacement of one or more amino acids within the amino acid sequence of the CD137 intracellular domain. In some embodiments, the mutant CD137 intracellular domain described herein is a CD137 intracellular domain as described herein, comprising a deletion or substitution of one or more amino acids within the amino acid sequence of the CD137 intracellular domain that can be targets for ubiquitination. In some embodiments, the mutant CD137 intracellular domain described herein is a CD137 protein as described herein, comprising a deletion or substitution of one or more lysine residues within the amino acid sequence of the CD137 intracellular domain that can be targets for ubiquitination. In some embodiments, the mutant CD137 intracellular domain described herein is a CD137 protein as described herein, comprising a deletion or substitution of one, two, three or four lysine residues within the amino acid sequence of the CD137 intracellular domain that can be targets for ubiquitination. In some embodiments, the lysine residues within the amino acid sequence of the CD137 intracellular domain described herein, that can be deleted or substituted are at amino acid positions 214, 218, 219 and/or 225 of the CD137 intracellular domain. In some embodiments, the mutant CD137 intracellular domain described herein is a CD137 protein as described herein, comprising a deletion or substitution of K214. In some embodiments, the mutant CD137 intracellular domain described herein is a CD137 protein as described herein, comprising a deletion or substitution of K218. In some embodiments, the mutant CD137 intracellular domain described herein is a CD137 protein as described herein, comprising a deletion or substitution of K219. In some embodiments, the mutant CD137 intracellular domain described herein is a CD137 protein as described herein, comprising a deletion or substitution of K225.

In some embodiments, the mutant CD137 intracellular domain can be a truncated CD137 intracellular domain. A truncated CD137 intracellular domain as described herein can be any one of the CD137 proteins described herein, in which a continuous stretch of more than one, two, three, four, five, six, seven, eight, nine, ten, eleven, twelve, thirteen, fourteen, fifteen, twenty, twenty-five, fifty, hundred, two hundred or more amino acids are deleted from the N-terminus the CD137 protein as described herein. A truncated CD137 intracellular domain as described herein can be any one of the CD137 intracellular domain sequences described herein, in which a continuous stretch of more than one, two, three, four, five, six, seven, eight, nine, ten or more amino acids are deleted from the N-terminus the CD137 intracellular domain as described herein. In some embodiments, the amino acids deleted from the N-terminus the CD137 intracellular domain includes one or more proximal polybasic amino acids of the CD137 intracellular domain.

In some embodiments, the mutant CD137 intracellular domain can be a truncated CD137 intracellular domain. In some embodiments, the truncated CD137 intracellular domain comprises an amino acid sequence according to amino acid position 13 to amino acid position 42 of the CD137 intracellular domain, of the present disclosure. In some embodiments, the truncated CD137 intracellular domain comprises a deletion of a continuous stretch of one, two, three, four, five, six, seven, eight, nine, ten or more amino acids from the N-terminus of the CD137 intracellular domain, of the present disclosure. In some embodiments, the truncated CD137 intracellular domain comprises a deletion of one, two, three, four, five, six, seven, eight, nine, ten or more amino acids from amino acid position 1 to amino acid position 12 of the N-terminus of the CD137 intracellular domain, of the present disclosure. In some embodiments, the truncated CD137 intracellular domain comprises a deletion of amino acid position 1 to amino acid position 12 of the N-terminus of the CD137 intracellular domain, of the present disclosure. In some embodiments, the CD137 intracellular domain comprises an amino acid sequence according to SEQ ID NO: 3.

In some embodiments, the mutant CD137 intracellular domain comprises a deletion of one or more proximal basic amino acids from amino acid position 1 to amino acid position 12 of the N-terminus of the CD137 intracellular domain, of the present disclosure. In some embodiments, the mutant CD137 intracellular domain comprises one or more proximal basic amino acid mutation(s) from amino acid position 1 to amino acid position 12 of the N-terminus of the CD137 intracellular domain, of the present disclosure. In some embodiments, the mutant CD137 intracellular domain comprises one or more proximal basic amino acid mutation(s) at amino acid positions selected from amino acid positions 1, 2, 3, 4, 5 and 6 of the N-terminus of the CD137 intracellular domain, of the present disclosure. In some embodiments, the mutant CD137 intracellular domain comprising one or more proximal basic amino acid mutation(s) of the present disclosure, further comprises a lysine mutation at amino acid position 12 of the N-terminus of the CD137 intracellular domain, of the present disclosure. In some embodiments, the lysine mutation is a lysine to alanine mutation, e.g., K12A. In some embodiments, the CD137 intracellular domain comprises an amino acid sequence according to SEQ ID NO: 3. In some embodiments, the truncated CD137 intracellular domain comprises an amino acid sequence according to SEQ ID NO: 3. In some embodiments, the truncated CD137 intracellular domain comprises an amino acid sequence having at least 50%, 55%, 60%, 65%, 70%, 75%, 80%, 85%, 90%, 95%, 97% or 99% identity to SEQ ID NO: 3.

In some embodiments, the second signal transduction domain of the BCMA-CAR disclosed herein comprises a truncated CD137 intracellular domain comprising an amino acid sequence according to SEQ ID NO: 3. In some embodiments, the second signal transduction domain of the BCMA-CAR disclosed herein is a truncated CD137 intracellular domain comprising an amino acid sequence having at least 50%, 55%, 60%, 65%, 70%, 75%, 80%, 85%, 90%, 95%, 97% or 99% identity to according to SEQ ID NO: 3.

### CD3ζ Domain

In some embodiments of the BCMA-CAR disclosed herein, the third signal transduction domain is derived from a CD3ζ domain.

In some embodiments, the third signal transduction domain of the BCMA-CAR disclosed herein is a truncated CD3ζ domain comprising an amino acid sequence according to SEQ ID NO: 4. In some embodiments, the third signal transduction domain of the BCMA-CAR disclosed herein, the third signal transduction domain of the BCMA-CAR disclosed herein is a truncated CD3ζ domain comprising an amino acid sequence having at least 50%, 55%, 60%, 65%, 70%, 75%, 80%, 85%, 90%, 95%, 97% or 99% identity to according to SEQ ID NO: 4.

### Ribosome Skipping Sequences

In some embodiments, a BCMA-CAR of the disclosure is multicistronic, i.e., comprises more than one distinct polypeptide produced from a single mRNA transcript. Sequences can be multicistronic by using various linkers, e.g., a polynucleotide sequence encoding a first molecule can be linked to a nucleotide sequence encoding a second molecule, e.g., 5′ first gene: linker: second gene 3′. A linker can encode a 2A ribosome skipping element, such as P2A. In some embodiments, a P2A linker comprises an amino acid sequence of SEQ ID NO: 16. In certain embodiments, the P2A linker is encoded by a nucleotide sequence comprising SEQ ID NO: 18. Other 2A ribosome skipping elements include, but are not limited to, E2A, T2A, and F2A. Such 2A ribosome skipping elements allow production of separate polypeptides encoded by the first and second genes produced during translation.

### Exemplary BCMA-CAR

Exemplary BCMA-CARs of the disclosure and amino acid sequences of the domains are shown in Table 1.

TABLE 1
Receptor 1 BCMA-CAR domains and amino acid sequences

| Domains | Amino Acid Sequence | SEQ ID NO |
|---|---|---|
| CD8a leader or | MALPVTALLLPLALLLHAARP | SEQ ID NO: 6 |
| CD8a signal peptide | | |
| BCMA binding domain or | EVQLQASGGGLAQPGGSLRLSCAASGRTFSTYF | SEQ ID NO: 8 |
| BCMA-nanobody | MAWFRQPPGKGLEYVGGIRWSDGVPHYADSVKG | |
| | RFTISRDNAKNTVYLQMNSLRAEDTAVYFCASR | |
| | GIADGSDFGSYGQGTQVTVSS | |
| CD8a hinge | TTTPAPRPPTPAPTIASQPLSLRPEACRPAAGG | SEQ ID NO: 7 |
| | AVHTRGLDFAC | |
| Extracellular domain | MALPVTALLLPLALLLHAARPEVQLQASGGGLA | SEQ ID NO: 1 |
| (CD8a leader/signal | QPGGSLRLSCAASGRTFSTYFMAWFRQPPGKGL | |
| peptide underlined, CD8a | EYVGGIRWSDGVPHYADSVKGRFTISRDNAKNT | |
| hinge in italic, and ICOS | VYLQMNSLRAEDTAVYFCASRGIADGSDFGSYG | |
| extracellular stalk in italic | QGTQVTVSSTTTPAPRPPTPAPTIASOPLSLRP | |
| and underlined) | EACRPAAGGAVHTRGLDFACSQLCCQLK | |
| Partial ICOS extracellular | SQLCCQLK | SEQ ID NO: 9 |
| stalk region | | |
| ICOS transmembrane | FWLPIGCAAFVVVCILGCILI | SEQ ID NO: 10 |
| domain | | |
| ICOS intracellular domain | CWLTKKKYSSSVHDPNGEYMFMRAVNTAKKSRL | SEQ ID NO: 2 |
| | TDVTL | |
| Truncated CD137 (4-1BB) | QPFMRPVQTTQEEDGCSCRFPEEEEGGCEL | SEQ ID NO: 3 |
| intracellular domain | | |
| Truncated CD3ζ domain | RVKFSRSADAPAYQQGQNQLYNELNLGRREEYD | SEQ ID NO: 4 |
| | VLDKRRGRDPEMGGK | |
| Receptor 1 BCMA-CAR | MALPVTALLLPLALLLHAARPEVQLQASGGGLA | SEQ ID NO: 5 |
| full sequence (also | QPGGSLRLSCAASGRTFSTYFMAWFRQPPGKGL | |
| referred to as NPB5005- | EYVGGIRWSDGVPHYADSVKGRFTISRDNAKNT | |
| ICOSBBtZt) | VYLQMNSLRAEDTAVYFCASRGIADGSDFGSYG | |
| | QGTQVTVSSTTTPAPRPPTPAPTIASQPLSLRP | |
| | EACRPAAGGAVHTRGLDFACSQLCCQLKFWLPI | |
| | GCAAFVVVCILGCILICWLTKKKYSSSVHDPNG | |
| | EYMFMRAVNTAKKSRLTDVTLQPFMRPVQTTQE | |
| | EDGCSCRFPEEEEGGCELRVKFSRSADAPAYQQ | |
| | GQNQLYNELNLGRREEYDVLDKRRGRDPEMGGK | |

In some embodiments, the BCMA-CAR of the present disclosure is encoded by the following nucleotide sequence (“clinical vector format”).

(SEQ ID NO: 11)
ATGGCTCTGCCTGTGACAGCTCTGCTGCTGCCTCTGGCTCTGCTTCTGC
ATGCCGCTAGACCTGAAGTGCAGTTGCAGGCTTCTGGCGGAGGACTTGC
TCAACCTGGCGGAAGCCTGAGACTGTCTTGTGCCGCCTCTGGCAGAACC
TTCAGCACCTACTTCATGGCCTGGTTCAGACAGCCTCCTGGCAAAGGCC
TGGAATACGTTGGCGGAATCCGTTGGAGTGATGGCGTGCCACACTACGC
CGATAGCGTGAAGGGCAGATTCACCATCAGCCGGGACAACGCCAAGAAC
ACCGTGTACCTCCAGATGAACAGCCTGAGAGCCGAGGATACCGCCGTGT
ACTTCTGTGCCAGCAGAGGAATCGCCGACGGCAGCGATTTTGGCTCTTA
TGGCCAGGGCACCCAAGTGACCGTGTCCAGCACAACAACCCCTGCTCCT
AGACCTCCTACACCAGCTCCTACAATCGCCAGCCAGCCTCTGTCTCTGA
GGCCAGAGGCTTGTAGACCTGCTGCTGGCGGAGCCGTGCATACAAGAGG
ACTGGATTTCGCCTGCAGCCAGCTGTGCTGTCAGCTGAAGTTCTGGCTG
CCTATCGGCTGCGCCGCCTTTGTGGTTGTGTGTATCCTGGGCTGCATCC
TGATCTGCTGGCTGACCAAGAAAAAGTACAGCAGCAGCGTGCACGACCC
CAACGGCGAGTACATGTTCATGAGAGCCGTGAACACCGCCAAGAAGTCC
AGACTGACCGACGTGACCCTCCAGCCTTTCATGAGGCCTGTGCAGACCA
CACAAGAAGAGGACGGCTGCTCCTGTCGGTTCCCCGAGGAAGAGGAAGG
CGGTTGCGAGCTGAGAGTGAAGTTCAGCAGATCCGCCGACGCTCCTGCC
TATCAGCAGGGCCAAAACCAGCTGTACAACGAGCTGAACCTGGGGAGAA
GAGAAGAGTACGACGTGCTGGACAAGCGGAGAGGCAGAGATCCTGAAAT
GGGCGGCAAATGA

TABLE 2
Control BCMA-CAR domains and corresponding amino acid sequences

| BCMA-CAR | Amino Acid Sequence |
|---|---|
| NPB5005-28Z | MALPVTALLLPLALLLHAARPEVQLQASGGGLAQPGGS |
| (SEQ ID NO: 12) | LRLSCAASGRTFSTYFMAWFRQPPGKGLEYVGGIRWSD |
| | GVPHYADSVKGRFTISRDNAKNTVYLQMNSLRAEDTAV |
| | YFCASRGIADGSDFGSYGQGTQVTVSSTTTPAPRPPTPAP |
| | TIASQPLSLRPEACRPAAGGAVHTRGLDFACLFPGPSKPF |
| | WVLVVVGGVLACYSLLVTVAFIIFWVRSKRSRLLHSDY |
| | MNMTPRRPGPTRKHYQPYAPPRDFAAYRSRVKFSRSAD |
| | APAYQQGQNQLYNELNLGRREEYDVLDKRRGRDPEMG |
| | GKPQRRKNPQEGLYNELQKDKMAEAYSEIGMKGERRR |
| | GKGHDGLYQGLSTATKDTYDALHMQALPPR |
| NPB5005-BBZ | MALPVTALLLPLALLLHAARPEVQLQASGGGLAQPGGS |
| (SEQ ID NO: 13) | LRLSCAASGRTFSTYFMAWFRQPPGKGLEYVGGIRWSD |
| | GVPHYADSVKGRFTISRDNAKNTVYLQMNSLRAEDTAV |
| | YFCASRGIADGSDFGSYGQGTQVTVSSTTTPAPRPPTPAP |
| | TIASQPLSLRPEACRPAAGGAVHTRGLDFACDIYIWAPL |
| | AGTCGVLLLSLVITLYCKRGRKKLLYIFKQPFMRPVQTT |
| | QEEDGCSCRFPEEEEGGCELRVKFSRSADAPAYQQGQN |
| | QLYNELNLGRREEYDVLDKRRGRDPEMGGKPQRRKNP |
| | QEGLYNELQKDKMAEAYSEIGMKGERRRGKGHDGLYQ |
| | GLSTATKDTYDALHMQALPPR |
| NPB5005-ICOSBBZ | MALPVTALLLPLALLLHAARPEVQLQASGGGLAQPGGS |
| (SEQ ID NO: 14) | LRLSCAASGRTFSTYFMAWFRQPPGKGLEYVGGIRWSD |
| | GVPHYADSVKGRFTISRDNAKNTVYLQMNSLRAEDTAV |
| | YFCASRGIADGSDFGSYGQGTQVTVSSTTTPAPRPPTPAP |
| | TIASQPLSLRPEACRPAAGGAVHTRGLDFACSQLCCQLK |
| | FWLPIGCAAFVVVCILGCILICWLTKKKYSSSVHDPNGE |
| | YMFMRAVNTAKKSRLTDVTLKRGRKKLLYIFKQPFMRP |
| | VQTTQEEDGCSCRFPEEEEGGCELRVKFSRSADAPAYQQ |
| | GQNQLYNELNLGRREEYDVLDKRRGRDPEMGGKPQRR |
| | KNPQEGLYNELQKDKMAEAYSEIGMKGERRRGKGHDG |
| | LYQGLSTATKDTYDALHMQALPPR |
| NPB5005-ICOSBBtZ | MALPVTALLLPLALLLHAARPEVQLQASGGGLAQPGGS |
| (SEQ ID NO: 15) | LRLSCAASGRTFSTYFMAWFRQPPGKGLEYVGGIRWSD |
| | GVPHYADSVKGRFTISRDNAKNTVYLQMNSLRAEDTAV |
| | YFCASRGIADGSDFGSYGQGTQVTVSSTTTPAPRPPTPAP |
| | TIASQPLSLRPEACRPAAGGAVHTRGLDFACSQLCCQLK |
| | FWLPIGCAAFVVVCILGCILICWLTKKKYSSSVHDPNGE |
| | YMFMRAVNTAKKSRLTDVTLQPFMRPVQTTQEEDGCSC |
| | RFPEEEEGGCELRVKFSRSADAPAYQQGQNQLYNELNL |
| | GRREEYDVLDKRRGRDPEMGGKPQRRKNPQEGLYNEL |
| | QKDKMAEAYSEIGMKGERRRGKGHDGLYQGLSTATKD |
| | TYDALHMQALPPR |
| FHVH33_CD8_BB_Z | MALPVTALLLPLALLLHAARPEVQLLESGGGLVQPGGSL |
| (SEQ ID NO: 22) | RLSCAASGFTFSSYAMSWVRQAPGKGLEWVSSISGSGD |
| | YIYYADSVKGRFTISRDISKNTLYLQMNSLRAEDTAVYY |
| | CAKEGTGANSSLADYRGQGTLVTVSSFVPVFLPAKPTTT |
| | PAPRPPTPAPTIASQPLSLRPEACRPAAGGAVHTRGLDFA |
| | CDIYIWAPLAGTCGVLLLSLVITLYCNHRNKRGRKKLLY |
| | IFKQPFMRPVQTTQEEDGCSCRFPEEEEGGCELRVKFSRS |
| | ADAPAYQQGQNQLYNELNLGRREEYDVLDKRRGRDPE |
| | MGGKPQRRKNPQEGLYNELQKDKMAEAYSEIGMKGER |
| | RRGKGHDGLYQGLSTATKDTYDALHMQALPPR |
| LCAR-B38M_CD8_BB_Z | MALPVTALLLPLALLLHAARPQVKLEESGGGLVQAGRS |
| (SEQ ID NO: 23) | LRLSCAASEHTFSSHVMGWFRQAPGKERESVAVIGWRD |
| | ISTSYADSVKGRFTISRDNAKKTLYLQMNSLKPEDTAVY |
| | YCAARRIDAADFDSWGQGTQVTVSSGGGGSGGGGSGG |
| | GGSGGGGSGGGGSAVQLVESGGGLVQAGDSLRLTCTAS |
| | GRAFSTYFMAWFRQAPGKEREFVAGIAWSGGSTAYADS |
| | VKGRFTISRDNAKNTVYLQMNSLKSEDTAVYYCASRGI |
| | EVEEFGAWGQGTQVTVSSTSTTTPAPRPPTPAPTIASQPL |
| | SLRPEACRPAAGGAVHTRGLDFACDIYIWAPLAGTCGVL |
| | LLSLVITLYCKRGRKKLLYIFKQPFMRPVQTTQEEDGCS |
| | CRFPEEEEGGCELRVKFSRSADAPAYQQGQNQLYNELN |
| | LGRREEYDVLDKRRGRDPEMGGKPQRRKNPQEGLYNE |
| | LQKDKMAEAYSEIGMKGERRRGKGHDGLYQGLSTATK |
| | DTYDALHMQALPPR |
| NPB5005_28_BB_Z | MALPVTALLLPLALLLHAARPEVQLQASGGGLAQPGGS |
| (SEQ ID NO: 24) | LRLSCAASGRTFSTYFMAWFRQPPGKGLEYVGGIRWSD |
| | GVPHYADSVKGRFTISRDNAKNTVYLQMNSLRAEDTAV |
| | YFCASRGIADGSDFGSYGQGTQVTVSSTTTPAPRPPTPAP |
| | TIASQPLSLRPEACRPAAGGAVHTRGLDFACLFPGPSKPF |
| | WVLVVVGGVLACYSLLVTVAFIIFWVRSKRSRLLHSDY |
| | MNMTPRRPGPTRKHYQPYAPPRDFAAYRSKRGRKKLLY |
| | IFKQPFMRPVQTTQEEDGCSCRFPEEEEGGCELRVKFSRS |
| | ADAPAYQQGQNQLYNELNLGRREEYDVLDKRRGRDPE |
| | MGGKPQRRKNPQEGLYNELQKDKMAEAYSEIGMKGER |
| | RRGKGHDGLYQGLSTATKDTYDALHMQALPPR |
| NPB5005_28BBt_Z | MALPVTALLLPLALLLHAARPEVQLQASGGGLAQPGGS |
| (SEQ ID NO: 25) | LRLSCAASGRTFSTYFMAWFRQPPGKGLEYVGGIRWSD |
| | GVPHYADSVKGRFTISRDNAKNTVYLQMNSLRAEDTAV |
| | YFCASRGIADGSDFGSYGQGTQVTVSSTTTPAPRPPTPAP |
| | TIASQPLSLRPEACRPAAGGAVHTRGLDFACLFPGPSKPF |
| | WVLVVVGGVLACYSLLVTVAFIIFWVRSKRSRLLHSDY |
| | MNMTPRRPGPTRKHYQPYAPPRDFAAYRSQPFMRPVQT |
| | TQEEDGCSCRFPEEEEGGCELRVKFSRSADAPAYQQGQN |
| | QLYNELNLGRREEYDVLDKRRGRDPEMGGKPQRRKNP |
| | QEGLYNELQKDKMAEAYSEIGMKGERRRGKGHDGLYQ |
| | GLSTATKDTYDALHMQALPPR |
| NPB5005_28_OX40t_Z | MALPVTALLLPLALLLHAARPEVQLQASGGGLAQPGGS |
| (SEQ ID NO: 26) | LRLSCAASGRTFSTYFMAWFRQPPGKGLEYVGGIRWSD |
| | GVPHYADSVKGRFTISRDNAKNTVYLQMNSLRAEDTAV |
| | YFCASRGIADGSDFGSYGQGTQVTVSSTTTPAPRPPTPAP |
| | TIASQPLSLRPEACRPAAGGAVHTRGLDFACLFPGPSKPF |
| | WVLVVVGGVLACYSLLVTVAFIIFWVRSKRSRLLHSDY |
| | MNMTPRRPGPTRKHYQPYAPPRDFAAYRSGGGSFRTPIQ |
| | EEQADAHSTLARVKFSRSADAPAYQQGQNQLYNELNLG |
| | RREEYDVLDKRRGRDPEMGGKPQRRKNPQEGLYNELQ |
| | KDKMAEAYSEIGMKGERRRGKGHDGLYQGLSTATKDT |
| | YDALHMQALPPR |
| NPB5005_ICOS_Z | MALPVTALLLPLALLLHAARPEVQLQASGGGLAQPGGS |
| (SEQ ID NO: 27) | LRLSCAASGRTFSTYFMAWFRQPPGKGLEYVGGIRWSD |
| | GVPHYADSVKGRFTISRDNAKNTVYLQMNSLRAEDTAV |
| | YFCASRGIADGSDFGSYGQGTQVTVSSTTTPAPRPPTPAP |
| | TIASQPLSLRPEACRPAAGGAVHTRGLDFACSQLCCQLK |
| | FWLPIGCAAFVVVCILGCILICWLTKKKYSSSVHDPNGE |
| | YMFMRAVNTAKKSRLTDVTLRVKFSRSADAPAYQQGQ |
| | NQLYNELNLGRREEYDVLDKRRGRDPEMGGKPQRRKN |
| | PQEGLYNELQKDKMAEAYSEIGMKGERRRGKGHDGLY |
| | QGLSTATKDTYDALHMQALPPR |
| NPB5005_ICOS_OX40t_Z | MALPVTALLLPLALLLHAARPEVQLQASGGGLAQPGGS |
| (SEQ ID NO: 28) | LRLSCAASGRTFSTYFMAWFRQPPGKGLEYVGGIRWSD |
| | GVPHYADSVKGRFTISRDNAKNTVYLQMNSLRAEDTAV |
| | YFCASRGIADGSDFGSYGQGTQVTVSSTTTPAPRPPTPAP |
| | TIASQPLSLRPEACRPAAGGAVHTRGLDFACSQLCCQLK |
| | FWLPIGCAAFVVVCILGCILICWLTKKKYSSSVHDPNGE |
| | YMFMRAVNTAKKSRLTDVTLGGGSFRTPIQEEQADAHS |
| | TLARVKFSRSADAPAYQQGQNQLYNELNLGRREEYDVL |
| | DKRRGRDPEMGGKPQRRKNPQEGLYNELQKDKMAEAY |
| | SEIGMKGERRRGKGHDGLYQGLSTATKDTYDALHMQA |
| | LPPR |
| NPB5005_28_Zt | MALPVTALLLPLALLLHAARPEVQLQASGGGLAQPGGS |
| (SEQ ID NO: 29) | LRLSCAASGRTFSTYFMAWFRQPPGKGLEYVGGIRWSD |
| | GVPHYADSVKGRFTISRDNAKNTVYLQMNSLRAEDTAV |
| | YFCASRGIADGSDFGSYGQGTQVTVSSTTTPAPRPPTPAP |
| | TIASQPLSLRPEACRPAAGGAVHTRGLDFACLFPGPSKPF |
| | WVLVVVGGVLACYSLLVTVAFIIFWVRSKRSRLLHSDY |
| | MNMTPRRPGPTRKHYQPYAPPRDFAAYRSRVKFSRSAD |
| | APAYQQGQNQLYNELNLGRREEYDVLDKRRGRDPEMGGK |
| NPB5005_BB_Zt | MALPVTALLLPLALLLHAARPEVQLQASGGGLAQPGGS |
| (SEQ ID NO: 30) | LRLSCAASGRTFSTYFMAWFRQPPGKGLEYVGGIRWSD |
| | GVPHYADSVKGRFTISRDNAKNTVYLQMNSLRAEDTAV |
| | YFCASRGIADGSDFGSYGQGTQVTVSSTTTPAPRPPTPAP |
| | TIASQPLSLRPEACRPAAGGAVHTRGLDFACDIYIWAPL |
| | AGTCGVLLLSLVITLYCKRGRKKLLYIFKQPFMRPVQTT |
| | QEEDGCSCRFPEEEEGGCELRVKFSRSADAPAYQQGQN |
| | QLYNELNLGRREEYDVLDKRRGRDPEMGGK |
| NPB5005_28_BBt_Zt | MALPVTALLLPLALLLHAARPEVQLQASGGGLAQPGGS |
| (SEQ ID NO: 31) | LRLSCAASGRTFSTYFMAWFRQPPGKGLEYVGGIRWSD |
| | GVPHYADSVKGRFTISRDNAKNTVYLQMNSLRAEDTAV |
| | YFCASRGIADGSDFGSYGQGTQVTVSSTTTPAPRPPTPAP |
| | TIASQPLSLRPEACRPAAGGAVHTRGLDFACLFPGPSKPF |
| | WVLVVVGGVLACYSLLVTVAFIIFWVRSKRSRLLHSDY |
| | MNMTPRRPGPTRKHYQPYAPPRDFAAYRSQPFMRPVQT |
| | TQEEDGCSCRFPEEEEGGCELRVKFSRSADAPAYQQGQN |
| | QLYNELNLGRREEYDVLDKRRGRDPEMGGK |
| NPB5005_28_OX40t_Zt | MALPVTALLLPLALLLHAARPEVQLQASGGGLAQPGGS |
| (SEQ ID NO: 32) | LRLSCAASGRTFSTYFMAWFRQPPGKGLEYVGGIRWSD |
| | GVPHYADSVKGRFTISRDNAKNTVYLQMNSLRAEDTAV |
| | YFCASRGIADGSDFGSYGQGTQVTVSSTTTPAPRPPTPAP |
| | TIASQPLSLRPEACRPAAGGAVHTRGLDFACLFPGPSKPF |
| | WVLVVVGGVLACYSLLVTVAFIIFWVRSKRSRLLHSDY |
| | MNMTPRRPGPTRKHYQPYAPPRDFAAYRSGGGSFRTPIQ |
| | EEQADAHSTLARVKFSRSADAPAYQQGQNQLYNELNLG |
| | RREEYDVLDKRRGRDPEMGGK |
| NPB5005_ICOS_OX40t_Zt | MALPVTALLLPLALLLHAARPEVQLQASGGGLAQPGGS |
| (SEQ ID NO: 33) | LRLSCAASGRTFSTYFMAWFRQPPGKGLEYVGGIRWSD |
| | GVPHYADSVKGRFTISRDNAKNTVYLQMNSLRAEDTAV |
| | YFCASRGIADGSDFGSYGQGTQVTVSSTTTPAPRPPTPAP |
| | TIASQPLSLRPEACRPAAGGAVHTRGLDFACSQLCCQLK |
| | FWLPIGCAAFVVVCILGCILICWLTKKKYSSSVHDPNGE |
| | YMFMRAVNTAKKSRLTDVTLGGGSFRTPIQEEQADAHS |
| | TLARVKFSRSADAPAYQQGQNQLYNELNLGRREEYDVL |
| | DKRRGRDPEMGGK |

In some aspects, provided herein is a BCMA-CAR comprising: (a) an extracellular domain comprising the amino acid sequence of SEQ ID NO: 1; (b) a transmembrane domain; and (c) a chimeric intracellular domain comprising a first, a second and at least a third signal transduction domain, wherein the first signal transduction domain comprises an ICOS intracellular domain according to SEQ ID NO: 2, the second signal transduction domain comprises a truncated CD137 (4-1BB) intracellular domain according to SEQ ID NO: 3, and the at least third signal transduction domain comprises a truncated CD3ζ domain according to SEQ ID NO: 4.

In some embodiments, the BCMA-CAR comprises (a) an extracellular domain comprising the amino acid sequence of SEQ ID NO: 1; (b) a transmembrane domain; and (c) a chimeric intracellular domain comprising a first, a second and a third signal transduction domains, wherein the first signal transduction domain comprises an ICOS intracellular domain according to SEQ ID NO: 2, the second signal transduction domain comprises a truncated CD137 (4-1BB) intracellular domain according to SEQ ID NO: 3, and the third signal transduction domain comprises a truncated CD3ζ domain according to SEQ ID NO: 4.

In some embodiments, the BCMA-CAR comprises: (a) an extracellular domain comprising the amino acid sequence of SEQ ID NO: 1; (b) a transmembrane domain, wherein the transmembrane domain comprises the amino acid sequence of SEQ ID NO: 10; and (c) a chimeric intracellular domain comprising a first, a second and at least a third signal transduction domain, wherein the first signal transduction domain comprises an ICOS intracellular domain according to SEQ ID NO: 2, the second signal transduction domain comprises a truncated CD137 (4-1BB) intracellular domain according to SEQ ID NO: 3, and the at least third signal transduction domain comprises a truncated CD3ζ domain according to SEQ ID NO: 4.

In some embodiments, the BCMA-CAR comprises: (a) an extracellular domain comprising the amino acid sequence of SEQ ID NO: 1; (b) a transmembrane domain, wherein the transmembrane domain comprises the amino acid sequence of SEQ ID NO: 10; and (c) a chimeric intracellular domain comprising a first, a second and a third signal transduction domains, wherein the first signal transduction domain comprises an ICOS intracellular domain according to SEQ ID NO: 2, the second signal transduction domain comprises a truncated CD137 (4-1BB) intracellular domain according to SEQ ID NO: 3, and the third signal transduction domain comprises a truncated CD3ζ domain according to SEQ ID NO: 4.

In some embodiments, the BCMA-CAR comprises an extracellular domain comprising the amino acid sequence of SEQ ID NO: 1. In some embodiments, the BCMA-CAR comprises a transmembrane domain comprising the amino acid sequence of SEQ ID NO: 10. In some embodiments, the BCMA-CAR comprises a first signal transduction domain comprising the amino acid sequence of SEQ ID NO: 2, a second signal transduction domain comprising the amino acid sequence of SEQ ID NO: 3, and a third signal transduction domain comprising the amino acid sequence of SEQ ID NO: 4.

In some embodiments, the BCMA-CAR extracellular domain comprises a signal peptide domain comprising the amino acid sequence of SEQ ID NO: 6. In some embodiments, the extracellular domain comprises a hinge domain comprising the amino acid sequence of SEQ ID NO: 7. In some embodiments, the extracellular domain comprises a BCMA-binding domain comprising the amino acid sequence of SEQ ID NO: 8. In some embodiments, the extracellular domain comprises an ICOS stalk comprising the amino acid sequence of SEQ ID NO: 9. In some embodiments, the BCMA-CAR extracellular domain comprises a signal peptide domain of the amino acid sequence of SEQ ID NO: 6. In some embodiments, the extracellular domain comprises a hinge domain of the amino acid sequence of SEQ ID NO: 7. In some embodiments, the extracellular domain comprises a BCMA-binding domain of the amino acid sequence of SEQ ID NO: 8. In some embodiments, the extracellular domain comprises an ICOS stalk of the amino acid sequence of SEQ ID NO: 9. In some embodiments, the BCMA-CAR comprises an extracellular domain comprising the amino acid sequence of SEQ ID NO: 1. In some embodiments, the BCMA-CAR comprises an extracellular domain of SEQ ID NO: 1.

In some embodiments, the BCMA-CAR comprises the amino acid sequence according to SEQ ID NO: 1. In some embodiments, the BCMA-CAR comprises the amino acid sequence having at least 50%, 55%, 60%, 65%, 70%, 75%, 80%, 85%, 90%, 95%, 9, 7% or 99% identity to SEQ ID NO: 1.

In some embodiments, the BCMA-CAR comprises an amino acid sequence of SEQ ID NO: 5. In some embodiments, the BCMA-CAR comprises an amino acid sequence having at least 50%, 55%, 60%, 65%, 70%, 75%, 80%, 85%, 90%, 95%, 97% or 99% identity to according to SEQ ID NO: 5. In some embodiments, the BCMA-CAR comprises an amino acid sequence having at least 50%, 55%, 60%, 65%, 70%, 75%, 80%, 85%, 90%, 95%, 97% or 99% identity to according to SEQ ID NO: 5, wherein the BCMA-binding domain comprises the amino acid sequence of SEQ ID NO: 8.

### Therapeutic Applications

Also provided herein are methods of using a BCMA-CAR of the present disclosure. The therapeutic method can be characterized according to the disease or condition, e.g., cancer, to be treated. The BCMA-CAR of the present disclosure is suitable for use in cancer, e.g., a cancer expressing BCMA.

In some embodiments, the cancer is a soft tissue sarcoma or a bone sarcoma (osteosarcoma). In some embodiments, the cancer is a sarcoma selected from vesicular rhabdomyosarcoma, vesicular soft tissue sarcoma, ameloblastoma, angiosarcoma, chondrosarcoma, chordoma, bright tissue sarcoma, dedifferentiated liposarcoma, Hyperplastic small round cell tumor of connective tissue, embryonic rhabdomyosarcoma, epithelioid fibrosarcoma, epithelioid hemangioendothelioma, epithelioid sarcoma; sensitive neuroblastoma (esthesioneuroblastoma), Ewing sarcoma, extrarenal rhabdomyosarcoma, extraosseous myxoid chondrosarcoma, extraosseous osteosarcoma, fibrosarcoma, giant cell tumor, hemangiopericytoma, infantile fibrosarcoma, inflammatory myofibroblastoma, Kaposi sarcoma, bone smooth muscle sarcoma, liposarcoma, osteosarcoma, malignant fibrous histiocytoma (MFH), malignant fibrous histiocytoma (MFH), malignant mesenchymal tumor, malignant peripheral nerve sheath tumor, mesenchymal chondrosarcoma, myxoid liposarcoma, myxoid inflammatory fibroblastic sarcoma, multiple tumors with perivascular epithelioid cell differentiation, osteosarcoma, extraperiosteal osteosarcoma, tumors with perivascular epithelial cell differentiation, periosteum osteosarcoma, polymorphic liposarcoma, polymorphic rhabdomyosarcoma, PNET/extraosseous Ewing's tumor, rhabdomyosarcoma, small cell osteosarcoma, single fibroids, synovial sarcoma or capillary dilated osteosarcoma.

In some embodiments, the cancer is a carcinoma selected from basal cell carcinoma, squamous cell carcinoma, renal cell carcinoma, ductal carcinoma in situ (DCIS), invasive ductal carcinoma or adenocarcinoma. In some embodiments, the cancer is a carcinoma selected from adenosquamous carcinoma, anaplastic carcinoma, large cell carcinoma, colorectal carcinoma, pancreatic carcinoma, nasopharyngeal carcinoma, or small cell carcinoma.

In some embodiments, the cancer is selected from anal cancer, appendix cancer; cholangiocarcinoma (i.e., biliary tract cancer), breast cancer, bladder cancer, brain tumor, breast cancer, cervical cancer, colon cancer, colorectal cancer, colon polyp, unidentified primary cancer (cup), esophagus cancer, eye cancer, tubal cancer, kidney cancer, liver cancer, lung cancer, medulloblastoma, melanoma, oral cancer, ovarian cancer, prostate cancer, pancreatic cancer, gastric cancer, testicular cancer, laryngeal cancer, thyroid cancer, uterine cancer, vaginal cancer, or vulvar cancer.

In some embodiments, the breast cancer is an invasive breast duct cancer, carcinoma in situ of the duct, invasive lobular carcinoma or lobular carcinoma in situ. In some embodiments, the pancreatic cancer is adenocarcinoma or islet cell carcinoma. In some embodiments, the colorectal cancer is adenocarcinoma. In some embodiments, colonic polyps are associated with familial adenomatous polyposis. In some embodiments, the bladder cancer is transitional cell bladder cancer, squamous cell bladder cancer, or adenocarcinoma. In some embodiments, the lung cancer is non-small cell lung cancer. In some embodiments, the non-small cell lung cancer is adenocarcinoma, squamous cell lung cancer, or large cell lung cancer. In some embodiments, the non-small cell lung cancer is large cell lung cancer. In some embodiments, the lung cancer is small cell lung cancer. In some embodiments, the prostate cancer is adenocarcinoma or small cell carcinoma. In some embodiments, the ovarian cancer is epithelial ovarian cancer. In some embodiments, the cholangiocarcinoma is proximal cholangiocarcinoma or distal cholangiocarcinoma.

In some embodiments, the cancer is any one of the hematological cancers selected from a leukemia, a myeloma, or a lymphoma. In some embodiments, the cancer is a leukemia selected from acute leukemia, acute lymphoblastic leukemia (ALL), acute lymphocytic leukemia, a B cell, T cell or FAB ALL, acute myeloid leukemia (AML), acute myelogenous leukemia, chronic myelocytic leukemia (CML), chronic lymphocytic leukemia (CLL), hairy cell leukemia, acute promyelocytic leukemia (APL), mixed-lineage leukemia (MLL) or myelodysplastic syndrome (MDS).

In some embodiments, the cancer is a myeloma, e.g., a multiple myeloma. In some embodiments, the cancer is a multiple myeloma selected from the hyperdiploid (HMM) or the non-hyperdiploid or hypodiploid subtypes of multiple myeloma. In some embodiments, the multiple myeloma is selected from light chain myeloma, non-secretory myeloma, solitary plasmacytoma, extramedullary plasmacytoma, monoclonal gammopathy of undetermined significance (MGUS), smoldering multiple myeloma (SMM), immunoglobulin D (IgD) myeloma, or immunoglobulin E (IgE) myeloma.

In some embodiments, the cancer is a lymphoma, e.g., a Hodgkin's lymphoma or a non-Hodgkin's lymphoma. In some embodiments, the cancer is a non-Hodgkin's lymphoma. In some embodiments, the cancer is a non-Hodgkin's lymphoma selected from a Small lymphocytic lymphoma (SLL), Lymphoplasmacytic lymphoma, Diffuse large cell lymphoma, Follicle center cell lymphoma, Burkitt's lymphoma, Burkitt-like lymphoma, Mantle cell lymphoma, or Marginal zone B cell lymphoma. In some embodiments, the cancer is a lymphoma, e.g., a Hodgkin's lymphoma. In some embodiments, the cancer is a Hodgkin's lymphoma selected from nodular sclerosis classical Hodgkin lymphoma, lymphocyte-rich classical Hodgkin lymphoma, or lymphocyte-depleted classical Hodgkin lymphoma.

In some embodiments, the cancer is any one of acute leukemia, acute lymphoblastic leukemia (ALL), acute lymphocytic leukemia, B cell, T cell or FAB ALL, acute myeloid leukemia (AML), acute myelogenous leukemia, chronic myelocytic leukemia (CML), chronic lymphocytic leukemia (CLL), hairy cell leukemia, myelodysplastic syndrome (MDS), Hodgkin's lymphoma, Hodgkin's disease, non-Hodgkin's lymphoma, multiple myeloma, colorectal carcinoma, pancreatic carcinoma, nasopharyngeal carcinoma, malignant histiocytosis, paraneoplastic syndrome/hypercalcemia of malignancy, bladder cancer, breast cancer, colorectal cancer, endometrial cancer, head cancer, neck cancer, hereditary nonpolyposis cancer, liver cancer, lung cancer, non-small cell lung cancer, ovarian cancer, pancreatic cancer, prostate cancer, renal cell carcinoma, testicular cancer, adenocarcinomas, sarcomas, malignant melanoma, and hemangioma.

In some embodiments, the extracellular domain of a B cell maturation antigen (BCMA) chimeric antigen receptor (BCMA-CAR) described herein binds to a target with a binding affinity of 1 fM to 100 μM. In some embodiments, the extracellular domain binds to a target with a binding affinity of 1 pM to 100 M. In some embodiments, the extracellular domain binds to a target with a binding affinity of 1 pM to 10 pM. In some embodiments, the extracellular domain binds to a target with a binding affinity of 10 pM to 50 pM. In some embodiments, the extracellular domain binds to a target with a binding affinity of 10 pM to 100 pM. In some embodiments, the extracellular domain binds to a target with a binding affinity of 100 pM to 500 pM. In some embodiments, the extracellular domain binds to a target with a binding affinity of 500 pM to 1 nM. In some embodiments, the extracellular domain binds to a target with a binding affinity of 1 nM to 10 nM. In some embodiments, the extracellular domain binds to a target with a binding affinity of 10 nM to 100 nM. In some embodiments, the extracellular domain binds to a target with a binding affinity of 100 nM to 500 nM. In some embodiments, the extracellular domain binds to a target with a binding affinity of 500 nM to 1 μM. In some embodiments, the extracellular domain binds to a target with a binding affinity of 1 μM to 10 μM. In some embodiments, the extracellular domain binds to a target with a binding affinity of 1 μM to 5 μM. In some embodiments, the extracellular domain binds to a target with a binding affinity of 5 μM to 7.5 pM. In some embodiments, the extracellular domain binds to a target with a binding affinity of 7.5 μM to 10 pM.

In some aspects, provided herein is also a BCMA CAR comprising: (a) an extracellular domain, wherein the extracellular domain has an amino acid sequence according to SEQ ID NO: 1; (b) a transmembrane domain; and (c) a chimeric intracellular domain comprising a first, a second and at least a third signal transduction domain, wherein the first signal transduction domain comprises an ICOS intracellular domain according to SEQ ID NO: 2, the second signal transduction domain comprises a truncated CD137 (4-1BB) intracellular domain according to SEQ ID NO: 3, and the at least third signal transduction domain comprises a truncated CD3ζ domain according to SEQ ID NO: 4. In some embodiments, the BCMA-CAR comprises an amino acid sequence according to SEQ ID NO: 5.

In some aspects, the present disclosure also provides a nucleic acid encoding the BCMA-CAR of the present disclosure. The present disclosure also provides a vector, e.g., an expression vector, comprising the nucleic acid of the present disclosure. In some embodiments, the nucleic acid or vector comprises SEQ ID NO:11. The present disclosure also provides a cell, e.g., an isolated cell, comprising the nucleic acid or the vector of the present disclosure. In some embodiments, the cell comprises a nucleic acid or vector comprising SEQ ID NO: 11.

### Expression on T Cells

In some embodiments, the BCMA-CAR disclosed herein is for expression in a T cell, wherein the T cell co-expresses at least one of the endogenous co-stimulatory molecules CD28, CD2, OX-40, ICOS, CD28, CD3, CD4, CD8, CD40L, or a combination thereof.

In some embodiments, the BCMA-CAR disclosed herein, is co-expressed with a T cell receptor (TCR) in a T cell. In some embodiments, the TCR is an endogenous TCR. In some embodiments, the TCR is an artificial TCR. In some embodiments, the artificial TCR is an affinity enhanced TCR. In some embodiments, the BCMA-CAR when co-expressed with a TCR in a T cell provides a second activation signal for inducing activation and proliferation of the T cell, wherein the first activation signal is provided by antigen binding by the TCR.

In some embodiments, the BCMA-CAR disclosed herein is expressed in a T cell as a component of an artificial receptor for a target. In some embodiments, the artificial receptor is a chimeric antigen receptor (CAR), a receptor for a ligand or a component thereof, an antibody or a fragment thereof. In some embodiments, the BCMA-CAR disclosed herein is expressed as a component of a CAR. In some embodiments, the BCMA-CAR disclosed herein is expressed as a component of an antibody or antigen-binding fragment thereof. In some embodiments, the antibody or antigen-binding fragment thereof is a Fab fragment, a F(ab)2 fragment, a diabody, a nanobody, a sdAb, a Fv, a VHH fragment, or a single chain Fv fragment. In some embodiments, the BCMA-CAR is expressed as a component of an artificial receptor in a T cell as disclosed herein and induces activation and/or proliferation of the T cell upon target binding by the artificial receptor.

In some embodiments, the modified T cell disclosed herein co-expresses at least one of the endogenous co-stimulatory molecules CD28, CD2, OX-40, ICOS, CD28, CD3, CD4, CD8, CD40L or a combination thereof.

In some embodiments, the method disclosed herein further comprises a modification of an endogenous sequence encoding a component of major histocompatibility complex (MHC) class I (MHC-I), wherein the modification reduces or eliminates a level of expression or activity of the MHC-I. In some embodiments, the modification reduces or eliminates the expression or activity of β2-macroglobulin.

The present disclosure also provides a composition comprising a BCMA-CAR disclosed herein. The present disclosure also provides a composition comprising a nucleic acid encoding the BCMA-CAR disclosed herein. The present disclosure also provides a composition comprising a vector comprising a nucleic acid disclosed herein. The present disclosure also provides a composition comprising a cell disclosed herein. The present disclosure also provides a composition comprising a T cell, e.g., a modified T cell, disclosed herein.

The present disclosure also provides a composition comprising a population of cells, wherein the population comprises a plurality of the cell comprising the nucleic acid encoding or a vector comprising the nucleic acid encoding a BCMA-CAR disclosed herein. The present disclosure also provides a composition comprising a population of cells, wherein the population comprises a plurality of the modified T cell disclosed herein.

The present disclosure also provides a method of producing a plurality of modified T cells, wherein the method comprises: a) providing a plurality of primary T cells disclosed herein; b) providing a composition comprising the BCMA-CAR disclosed herein, the nucleic acid encoding the BCMA-CAR disclosed herein, or the vector comprising the nucleic acid encoding the BCMA-CAR disclosed herein; and c) introducing into the plurality of primary T cells of (a) the composition of (b), to produce a plurality of modified T cells under conditions that stably express the BCMA-CAR within the plurality of modified T cells. In some embodiments, the method of producing a plurality of modified T cells disclosed herein, further comprises a step of modifying an endogenous sequence encoding an endogenous T cell Receptor (TCR), wherein the modification reduces or eliminates a level of expression or activity of the endogenous TCR. In some embodiments, the method of producing a plurality of modified T cells disclosed herein, further comprises a step of modifying an endogenous sequence, wherein the modification reduces or eliminates a level of expression or activity of a major histocompatibility complex (MHC) class I (MHC-I).

In some embodiments, the modifying an endogenous sequence encoding a T cell Receptor (TCR) uses a nucleic acid modifying system. In some embodiments, the modifying an endogenous sequence that reduces or eliminates a level of expression or activity uses a nucleic acid modifying system. In some embodiments, the nucleic acid modifying system comprises one or more of a CRISPR/Cas protein, a Transcription Activator-Like Effector Nuclease (TALEN), a Zinc Finger Nuclease (ZFN), and an endonuclease. In some embodiments, the modifying an endogenous sequence is done by nonhomologous end joining repair. In some embodiments, the nonhomologous end joining repair is generated by zinc finger nuclease introduced into the cell, e.g., by physical means, viral vector, or non-viral vector. In some embodiments, the nonhomologous end joining repair is generated by TALE nuclease (i.e., TALEN), introduced into the cell by physical means, viral vector, or non-viral vector. In some embodiments, the modifying an endogenous sequence encoding a T cell Receptor (TCR) reduces or eliminates a level of expression of the alpha chain of the TCR. In some embodiments, the modifying an endogenous sequence encoding a T cell Receptor (TCR) reduces or eliminates a level of expression of beta chain of the TCR. In some embodiments, the modifying an endogenous sequence encoding a T cell Receptor (TCR) reduces or eliminates a level of expression of both the alpha chain and the beta chain TCR alpha chain.

In some embodiments, the modifying an endogenous sequence that reduces or eliminates a level of expression or activity of a major histocompatibility complex (MHC) class I (MHC-I), wherein the modifying of an endogenous sequence reduces or eliminates a level of expression or activity of the MHC-I. In some embodiments, the modifying of an endogenous sequence reduces or eliminates the expression or activity of β2-macroglobulin.

In some embodiments, the method of producing a plurality of modified T cells disclosed herein, further comprises: d) maintaining or expanding the plurality of modified T cells in a suitable cell culture media; and e) either: i) cryopreserving the plurality of modified T cells in a suitable cell freezing media; or ii) preparing the plurality of modified T cells for administering to a subject suffering from a disease or disorder.

The compositions comprising the cells or modified T cells of the disclosure, and the plurality of modified T cells produced by the methods of the disclosure, intended for administration to a subject may be required to meet one or more “release criteria” that indicate that the composition is safe and efficacious for formulation as a pharmaceutical product and/or administration to a subject. Release criteria may include a requirement that a composition of the disclosure (e.g., a cell or modified T cell of the disclosure) comprises a particular percentage of cells or modified T cells expressing the BCMA-CAR of the disclosure on their cell surface. The expansion process should be continued until a specific criterion has been met (e.g., achieving a certain total number of cells or modified T cells of the disclosure or a certain percentage of total number of cells or modified T cells expressing the BCMA-CAR of the disclosure).

Certain criterion may signal a point at which the expansion process should end. For example, cells should be formulated, reactivated, or cryopreserved once they reach a cell size of 300 fL (otherwise, cells reaching a size above this threshold may start to die).

Cryopreservation immediately once a population of cells reaches an average cell size of less than 300 fL may yield better cell recovery upon thawing and culture because the cells have not yet reached a fully quiescent state prior to cryopreservation (a fully quiescent size is approximately 180 fL). Prior to expansion, T cells of the disclosure may have a cell size of about 180 fL but may more than quadruple their cell size (e.g., to approximately 900 fL) at 3 days post-expansion. Over the next 6-12 days of culture, the population of T cells may slowly decrease cell size to full quiescence at 180 fL.

A process for preparing a cell population for formulation may include, but is not limited to the steps of, concentrating the cells of the cell population, washing the cells, and/or further selection of the cells via drug resistance or magnetic bead sorting against a particular surface-expressed marker. A process for preparing a cell population for formulation may further include a sorting step to ensure the safety and purity of the final product. For example, if a tumor cell from a patient has been used to stimulate a modified T cell of the disclosure or that has been modified in order to stimulate a modified T cell of the disclosure that is being prepared for formulation, it is critical that no tumor cells from the patient are included in the final product.

In some embodiments, the cell disclosed herein, or the modified T cell disclosed herein, expresses on the cell surface the BCMA-CAR comprising a mutant CD137 intracellular signaling domain disclosed herein, at a level that is at least about 2×, 3×, 4×, 5×, 6×, 7×, 8×, 9×, 10× or 20×, more as compared to the level of expression of a co-stimulatory molecule comprising a wild type CD137 intracellular domain, respectively.

In some embodiments, the cell disclosed herein further comprises a sequence encoding an artificial antigen receptor, a therapeutic polypeptide, an immune cell modulatory protein, or a combination thereof. In some embodiments, the artificial antigen receptor comprises a chimeric antigen receptor (CAR). In some embodiments, the artificial antigen receptor comprises a recombinant T cell receptor (rTCR). In some embodiments, the artificial antigen receptor comprises an enhanced affinity TCR. In some embodiments, the artificial antigen receptor binds to a tumor associated antigen (TAA), a pathogen associated protein, or an antigen associated with the disease or disorder is a cancer, an autoimmune disease or disorder, an infectious disease, an inflammatory disease, a renal disease or disorder, a lung disease or disorder, a liver disease or disorder a neurodegenerative disorder or disorder, or a metabolic disorder or disorder.

In some embodiments, the artificial antigen receptor binds to a TAA associated with a solid tumor or a hematologic cancer. In some embodiments, artificial antigen receptor binds to a TAA associated with a cancer selected from any one of leukemia, acute leukemia, acute lymphoblastic leukemia (ALL), acute lymphocytic leukemia, B cell, T cell or FAB ALL, acute myeloid leukemia (AML), acute myelogenous leukemia, chronic myelocytic leukemia (CML), chronic lymphocytic leukemia (CLL), hairy cell leukemia, myelodysplastic syndrome (MDS), a lymphoma, Hodgkin's disease, a malignant lymphoma, non-Hodgkin's lymphoma, Burkitt's lymphoma, multiple myeloma, Kaposi's sarcoma, colorectal carcinoma, pancreatic carcinoma, nasopharyngeal carcinoma, malignant histiocytosis, paraneoplastic syndrome/hypercalcemia of malignancy, solid tumors, bladder cancer, breast cancer, colorectal cancer, endometrial cancer, head cancer, neck cancer, hereditary nonpolyposis cancer, Hodgkin's lymphoma, liver cancer, lung cancer, non-small cell lung cancer, ovarian cancer, pancreatic cancer, prostate cancer, renal cell carcinoma, testicular cancer, adenocarcinomas, sarcomas, malignant melanoma, and hemangioma.

In some embodiments, the artificial antigen receptor binds to a TAA selected from kallikrein 4, papillomavirus binding factor (PBF), preferentially expressed antigen of melanoma (PRAME), Wilms' tumor-I (WTI), Hydroxysteroid Dehydrogenase Like I (HSDLI), mesothelin, cancer testis antigen (NY-ES0-1), carcinoembryonic antigen (CEA), p53, human epidermal growth factor receptor 2/neuro receptor tyrosine kinase (Her2/Neu), carcinoma-associated epithelial cell adhesion molecule (EpCAM), ovarian and uterine carcinoma antigen (CAI25), folate receptor a, sperm protein 17, tumor-associated differentially expressed gene-12 (TADG-12), mucin-16 (MUC-16), LI cell adhesion molecule (LICAM), mannan-MUC-1, Human endogenous retrovirus K (HERV-K-MEL), Kita-kyushu lung cancer antigen-I (KK-LC-1), human cancer/testis antigen (KM-HN-1), cancer testis antigen (LAGE-I), melanoma antigen-Al (MAGE-AI), Sperm surface zona pellucida binding protein (Spl 7), Synovial Sarcoma, X Breakpoint 4 (SSX-4), Transient axonal glycoprotein-1 (TAG-I), Transient axonal glycoprotein-2 (TAG-2), Enabled Homolog (ENAH), mammoglobin-A, NY-BR-I, Breast Cancer Antigen, (BAGE-1), B melanoma antigen, melanoma antigen-Al (MAGE-AI), melanoma antigen-A2 (MAGE-A2), mucin k, synovial sarcoma, X breakpoint 2 (SSX-2), Taxol-resistance-associated gene-3 (TRAG-3), Avian Myelocytomatosis Viral Oncogene (c-myc), cyclin B 1, mucin I (MUC I), p62, survivin, lymphocyte common antigen (CD45), DickkopfWNT Signaling Pathway Inhibitor I (DKKI), telomerase, Kirsten rat sarcoma viral oncogene homolog (K-ras), G250, intestinal carboxyl esterase, alpha-fetoprotein, Macrophage Colony-Stimulating Factor (M-CSF), Prostate-specific membrane antigen (PSMA), caspase 5 (CASP-5), Cytochrome C Oxidase Assembly Factor I Homolog (COA-1), 0-linked □-N-acetylglucosamine transferase (OGT), Osteosarcoma Amplified 9, Endoplasmic Reticulum Lectin (OS-9), Transforming Growth Factor Beta Receptor 2 (TGF-betaRII), murine leukemia glycoprotein 70 (gp70), Calcitonin Related Polypeptide Alpha (CALCA), Programmed cell death 1 ligand 1 (CD274), Mouse Double Minute 2Homolog (mdm-2), alpha-actinin-4, elongation factor 2, Malic Enzyme 1 (MEI), Nuclear Transcription Factor Y Subunit C (NFYC), G Antigen 1,3 (GAGE-1,3), melanoma antigen-A6 (MAGE-A6), cancer testis antigen XAGE-lb, six transmembrane epithelial antigen of the prostate 1 (STEAPI), PAP, prostate specific antigen (PSA), Fibroblast Growth Factor 5 (FGF5), heat shock protein hsp70-2, melanoma antigen-A9 (MAGE-A9), Arg-specific ADP-ribosyltransferase family C (ARTCl), B-Raf Proto-Oncogene (B-RAF), Serine/Threonine Kinase, beta-catenin, Cell Division Cycle 27 homolog (Cdc27), cyclin dependent kinase 4 (CDK4), cyclin dependent kinase 12 (CDK12), Cyclin Dependent Kinase Inhibitor 2A (CDKN2A), Casein Kinase 1 Alpha 1 (CSNKIAI), Fibronectin 1 (FN1), Gruwih Anest Specific 7 (GAS7), Glycoprotein nonmetastatic melanoma protein B (GPNMB), HAUS Augmin Like Complex Subunit 3 (HAUS3), LDLR-fucosyltransferase, Melanoma Antigen Recognized By T cells 2 (MART2), myostatin (MSTN), Melanoma Associated Antigen (Mutated) 1 (MUM-1-2-3), Poly (A) polymerase gamma (neo-PAP), myosin class I, Protein phosphatase 1 regulatory subunit 3B (PPP1R3B), Peroxiredoxin-5 (PRDX5), Receptor-type tyrosine-protein phosphatase kappa (PTPRK), Transforming protein N-Ras (N-ras), retinoblastoma-associated factor 600 (RBAF600), sirtuin-2 (SIRT2), SNRPDI, triosephosphate isomerase, Ocular Albinism Type 1 Protein (OAI), member RAS oncogene family (RAB38), Tyrosinase related protein 1-2 (TRP-1-2), Melanoma Antigen Gp75 (gp75), tyrosinase, Melan-A (MART-1), Glycoprotein 100 melanoma antigen (gplOO), N-acetylglucosaminyltransferase V gene (GnTVf), Lymphocyte Antigen 6 Complex Locus K (LY6K), melanoma antigen-AlO (MAGE-AlO), melanoma antigen-Al2 (MAGE-Al2), melanoma antigen-C2 (MAGE-C2), melanoma antigen NA88-A, Taxol-resistant-associated protein 3 (TRAG-3), BDZ binding kinase (pbk), caspase 8 (CASP-8), sarcoma antigen 1 (SAGE), Breakpoint Cluster Region-Abelson oncogene (BCR-ABL), fusion protein in leukemia, dek-can, Elongation Factor Tu GTP Binding Domain Containing 2 (EFTUD2), ETS Variant gene 6/acute myeloid leukemia fusion protein (ETV6-AML1), FMS-like tyrosine kinase-3 internal tandem duplications (FLT3-ITD), cyclin-Al, Fibronectin Type III Domain Containing 3B (FDNC3B,) promyelocytic leukemia/retinoic acid receptor alpha fusion protein (pml-RARalpha), melanoma antigen-Cl (MAGE-Cl), membrane protein alternative spliced isoform (D393-CD20), melanoma antigen-A4 (MAGE-A4), and melanoma antigen-A3 (MAGE-A3).

In some embodiments, the artificial antigen receptor binds to an antigen associated with an autoimmune condition or disorder selected from any one of Type 1 Diabetes, rheumatoid arthritis (RA), systemic lupus erythematosis (SLE), or multiple sclerosis (MS). In some embodiments, the artificial antigen receptor binds to an antigen associated with an autoimmune condition or disorder selected from any one of Carboxypeptidase H, Chromogranin A, Glutamate decarboxylase, Imogen-38, Insulin, Insulinoma antigen-2 and 2β, Islet-specific glucose-6-phosphatase catalytic subunit related protein (IGRP), Proinsulin, α-enolase, Aquaporin-4, β-arrestin, Myelin basic protein, Myelin oligodendrocytic glycoprotein, Proteolipid protein, S100-β, Citrullinated protein, Collagen II, Heat shock proteins, Human cartilage glycoprotein, Double-stranded DNA, La antigen, Nucleosomal histones and ribonucleoproteins (snRNP), Phospholipid-β-2 glycoprotein I complex, Poly (ADP-ribose) polymerase, Sm antigens of U-1 small ribonucleoprotein complex.

In some embodiments, the artificial antigen receptor binds to a pathogen associated antigen from a bacterial, a fungal or a parasitic protein or fragment thereof. In some embodiments, the artificial antigen receptor binds to an antigen associated with HIV infection, human Cytomegalovirus infection, Hepatitis B infection, Hepatitis C infection, Ebola virus infection, Dengue, Yellow fever, Listeriosis, Tuberculosis, Cholera, Malaria, Leishmaniasis, or Trypanosoma infection, or a combination thereof.

In some embodiments, the artificial antigen receptor binds to an antigen associated with a neurodegenerative disorder or condition selected from Alzheimer's disease (AD) and other dementias, Parkinson's disease (PD) and PD-related disorders, Prion disease, Motor neuron diseases (MND), Huntington's disease (HD), Spinocerebellar ataxia (SCA) or Spinal muscular atrophy (SMA). In some embodiments, the antigen associated with the neurodegenerative disorder or condition is any one of Amyloid β (Aβ), tau, alpha-synuclein (α-syn), mHTT, or prion PrPsc, or a combination thereof.

In some embodiments, the therapeutic polypeptide is a cytokine, a cytokine receptor, a chemokine, a chemokine receptor, an immunogenic polypeptide, or a cell surface protein that binds to a target on the surface of another cell. In some embodiments, the immune cell modulatory protein is a cytokine, a chemokine, a transcription factor, a protein kinase, a protease, a component, or an adaptor protein of a cell signaling pathway.

In some embodiments, the cell disclosed herein expresses the BCMA-CAR disclosed herein. In some embodiments, the cell disclosed herein expresses the BCMA-CAR disclosed herein stably or transiently. In some embodiments, the cell disclosed herein expresses the BCMA-CAR disclosed herein stably. In some embodiments, the cell disclosed herein expresses the BCMA-CAR disclosed herein transiently.

In some embodiments, the cell disclosed herein co-expresses at least one of the endogenous co-stimulatory molecules CD28, CD2, OX-40, ICOS, CD28, CD3, CD4, CD8, CD40L, or a combination thereof.

The present disclosure also provides a modified T lymphocyte (T cell), comprising: (a) a modification of an endogenous sequence encoding a T cell Receptor (TCR), wherein the modification reduces or eliminates a level of expression or activity of the TCR and/or (b) a recombinant T cell co-stimulatory receptor (RTCR) disclosed herein. In some embodiments, the modification of an endogenous sequence encoding a T cell Receptor (TCR) is carried out using a nucleic acid modifying system. In some embodiments, the nucleic acid modifying system is one or more of a CRISPR/Cas protein, a Transcription Activator-Like Effector Nuclease (TALEN), a Zinc Finger Nuclease (ZFN), and an endonuclease. In some embodiments, the modification of an endogenous sequence encoding a T cell Receptor (TCR) is done by nonhomologous end joining repair. In some embodiments, the nonhomologous end joining repair is generated by zinc finger nuclease, introduced into the cell by physical means, viral vector, or non-viral vector. In some embodiments, the nonhomologous end joining repair is generated by TALE nuclease, introduced into the cell by physical means, viral vector, or non-viral vector. In some embodiments, the modification of an endogenous sequence encoding a T cell Receptor (TCR) reduces or eliminates a level of expression of the alpha chain of the TCR. In some embodiments, the modification of an endogenous sequence encoding a T cell Receptor (TCR) reduces or eliminates a level of expression of beta chain of the TCR. In some embodiments, the modification of an endogenous sequence encoding a T cell Receptor (TCR) reduces or eliminates a level of expression of both the alpha chain and the beta chain TCR alpha chain

### Pharmaceutical Composition or Formulation

In some embodiments, the compositions disclosed herein, and the population of modified T cells produced using the methods disclosed herein, is in the form of a pharmaceutical formulation (or composition). In some embodiments, the pharmaceutical formulation disclosed herein comprises a pharmaceutically acceptable carrier. A pharmaceutical formulation of the disclosure may be distributed into bags for infusion, cryopreservation, and/or storage.

A pharmaceutical formulation of the disclosure may be cryopreserved using a standard protocol and, optionally, an infusible cryopreservation medium. For example, a DMSO free cryopreservant (e.g., CryoSOfree™ DMSO-free Cryopreservation Medium) may be used to reduce freezing-related toxicity. A cryopreserved pharmaceutical formulation of the disclosure may be stored for infusion to a patient at a later date. An effective treatment may require multiple administrations of a pharmaceutical formulation of the disclosure and, therefore, pharmaceutical formulations may be packaged in pre-aliquoted “doses” that may be stored frozen but separated for thawing of individual doses.

A pharmaceutical formulation of the disclosure may be stored at room temperature. An effective treatment may require multiple administrations of a pharmaceutical formulation of the disclosure and, therefore, pharmaceutical formulations may be packaged in pre-aliquoted “doses” that may be stored together but separated for administration of individual doses.

A pharmaceutical formulation of the disclosure may be archived for subsequent re-expansion and/or selection for generation of additional doses to the same patient in the case of an allogenic therapy who may need an administration at a future date following, for example, a remission and relapse of a condition.

As noted above, the disclosure provides for stable formulations, which may comprise a phosphate buffer with saline or a chosen salt, as well as preserved solutions and formulations containing a preservative, or multi-use preserved formulations suitable for pharmaceutical and/or veterinary use, comprising at least one modified cell in a pharmaceutically acceptable formulation. Preserved formulations contain at least one known preservative or optionally selected from the group consisting of at least one phenol, m-cresol, p-cresol, o-cresol, chlorocresol, benzyl alcohol, phenylmercuric nitrite, phenoxyethanol, formaldehyde, chlorobutanol, magnesium chloride (e.g., hexahydrate), alkylparaben (methyl, ethyl, propyl, butyl and the like), benzalkonium chloride, benzethonium chloride, sodium dehydroacetate and thimerosal, polymers, or mixtures thereof in an aqueous diluent. Any suitable concentration or mixture can be used as known in the art, such as about 0.0015%, or any range, value, or fraction therein. Non-limiting examples include, no preservative, about 0.1-2% m-cresol (e.g., 0.2, 0.3. 0.4, 0.5, 0.9, 1.0%), about 0.1-3% benzyl alcohol (e.g., 0.5, 0.9, 1.1, 1.5, 1.9, 2.0, 2.5%), about 0.001-0.5% thimerosal (e.g., 0.005, 0.01), about 0.001-2.0% phenol (e.g., 0.05, 0.25, 0.28, 0.5, 0.9, 1.0%), 0.0005-1.0% alkylparaben(s) (e.g., 0.00075, 0.0009, 0.001, 0.002, 0.005, 0.0075, 0.009, 0.01, 0.02, 0.05, 0.075, 0.09, 0.1, 0.2, 0.3, 0.5, 0.75, 0.9, 1.0%), and the like.

As noted above, in some aspects the disclosure provides an article of manufacture, comprising packaging material and at least one vial comprising a solution of at least one modified cell with the prescribed buffers and/or preservatives, optionally in an aqueous diluent, wherein said packaging material comprises a label that indicates that such solution can be held over a period of 1, 2, 3, 4, 5, 6, 9, 12, 18, 20, 24, 30, 36, 40, 48, 54, 60, 66, 72 hours or greater.

The articles of manufacture of the present disclosure are useful for administration over a period ranging from immediate to twenty-four hours or greater. Accordingly, the presently claimed articles of manufacture offer significant advantages to the patient. Formulations of the disclosure can optionally be safely stored at temperatures of from about 2° C. to about 40° C. and retain the biological activity of the protein for extended periods of time, thus allowing a package label indicating that the solution can be held and/or used over a period of 6, 12, 18, 24, 36, 48, 72, or 96 hours or greater.

The products of the present disclosure can include packaging material. The packaging material provides, in addition to the information required by the regulatory agencies, the conditions under which the product can be used.

In another aspect, the present disclosure also provides a method of treating a disease or disorder, comprising administering to a subject in need thereof a therapeutically effective number of the cell comprising the nucleic acid encoding or the vector comprising the nucleic acid encoding a CAR disclosed herein, a therapeutically effective number of any one of the modified T cell disclosed herein, a therapeutically effective amount of any one of the compositions disclosed herein, or a therapeutically effective number of the plurality of modified T cells produced by the method disclosed herein. A BCMA-CAR as provided herein can be used in a method of treating a disease or disorder expressing BCMA, e.g., a cancer-expressing BCMA.

In some embodiments, the subject is a mammal. In some embodiments, the mammal is any one of a human, a primate, a rodent, a canine, a feline, an ungulate, an equine, and a porcine. In some embodiments, the mammal is a human. In some embodiments, the mammal is a non-human primate. In some embodiments, the disease or disorder is any one of a cancer, an autoimmune disorder, an infectious disease, an inflammatory disease or condition, a renal disease or disorder, a lung disease or disorder, a liver disease or disorder, a cardiovascular system disease or disorder, a neurodegenerative disorder or condition, or a metabolic disorder or condition. In some embodiments, the cancer is a solid tumor or a hematologic cancer. In some embodiments, the infectious disease is caused by a bacterium, a virus, a fungus, a protozoa, or a parasite. In some embodiments, the neurodegenerative disorder or condition is any one of Alzheimer's disease (AD) and other dementias, Parkinson's disease (PD) and PD-related disorders, Prion disease, Motor neuron diseases (MND), Huntington's disease (HD), Spinocerebellar ataxia (SCA) or Spinal muscular atrophy (SMA).

The following examples are provided to better illustrate the present disclosure and are not to be interpreted as limiting the scope of the disclosure. To the extent that specific materials are mentioned, it is merely for purposes of illustration and is not intended to limit the disclosure. One skilled in the art may develop equivalent means or reactants without the exercise of inventive capacity and without departing from the scope of the disclosure.

### EXAMPLES

### Example 1: Materials and Methods

### Media and Cell Lines

DMEM was supplemented with Pen/Strep/Glutamine, 20 mM HEPES, 10 μg/mL Gentamycin and 10% FBS to make complete DMEM. RPMI was supplemented with Pen/Strep/Glutamine, 20 mM HEPES, 10 μg/mL Gentamycin, 10% FBS, and 50 μM 2-ME to make complete RPMI. T cell growth media was made by supplementing complete RPMI with 50 ng/ml IL2, 10 ng/ml IL7, and 10 ng/mL IL15 (Peprotech). X-Vivo 15™ (Lonza®) was supplemented with 1% Human Serum, 20 mM HEPES, Pen/Strep/Glutamine, and 10 μg/mL Gentamycin to make Cytokine Media. Human peripheral blood mononuclear cells (PBMCs) were purchased from iSpecimen® and cultured in complete RPMI. 293 FT were purchased from Invitrogen®. K562 and A375 cells were purchased from ATCC® and cultured in complete DMEM.

### Plasmids and Cloning

A lentiviral plasmid containing the PGK promoter driving a truncated human EGFR receptor (huEGFRt) followed by the MSCV promoter driving GFP and a subsequent WPRE sequence was ordered from VectorBuilder. Co-stimulatory molecules followed by a P2A sequence were ordered as a single gene block (Invitrogen) and placed in frame with the huEGFRt sequence using NEB® builder homology-based recombination. CAR sequences were constructed from gene block fragments (Invitrogen®) and cloned with NEB® builder downstream of the MSCV promoter following GFP excision. PD-L1_P2A and HLA-A2 were cloned in frame with the huEGFRt and in place of GFP, respectively.

P2A amino acid sequence
(SEQ ID NO: 16)
GSGATNFSLLKQAGDVEENPGP
Human EGFRt amino acid sequence
(Other name: huEGFRt (AA112))
(SEQ ID NO: 17)
MLLLVTSLLLCELPHPAFLLIPRKVCNGIGIGEFKDSLSINATNIKHF
KNCTSISGDLHILPVAFRGDSFTHTPPLDPQELDILKTVKEITGFLLI
QAWPENRTDLHAFENLEIIRGRTKQHGQFSLAVVSLNITSLGLRSLKE
ISDGDVIISGNKNLCYANTINWKKLFGTSGQKTKIISNRGENSCKATG
QVCHALCSPEGCWGPEPRDCVSCRNVSRGRECVDKCNLLEGEPREFVE
NSECIQCHPECLPQAMNITCTGRGPDNCIQCAHYIDGPHCVKTCPAGV
MGENNTLVWKYADAGHVCHLCHPNCTYGCTGPGLEGCPTNGPKIPSIA
TGMVGALLLLLVVALGIGLFM
P2A nucleic acid sequence
(SEQ ID NO: 18)
GGATCCGGCGCCACCAATTTCAGCCTGCTGAAACAGGCTGGCGACGTG
GAAGAGAACCCTGGACCT
Human EGFRt nucleic acid sequence
(SEQ ID NO: 19)
ATGCTGCTGCTGGTTACATCTCTGCTGCTGTGCGAGCTGCCCCATCCT
GCCTTTCTGCTGATCCCCAGAAAAGTGTGCAACGGCATCGGCATCGGA
GAGTTCAAGGACAGCCTGAGCATCAACGCCACCAACATCAAGCACTTC
AAGAACTGCACCAGCATCAGCGGCGACCTGCACATTCTGCCTGTGGCC
TTTAGAGGCGACAGCTTCACCCACACACCTCCACTGGACCCTCAAGAG
CTGGACATCCTGAAAACCGTGAAAGAGATCACCGGATTTCTGTTGATC
CAGGCTTGGCCCGAGAACCGGACAGATCTGCACGCCTTCGAGAACCTG
GAAATCATCAGAGGCCGGACCAAGCAGCACGGCCAGTTTTCTCTGGCT
GTGGTGTCCCTGAACATCACCAGCCTGGGCCTGAGAAGCCTGAAAGAA
ATCAGCGACGGCGACGTGATCATCTCCGGCAACAAGAACCTGTGCTAC
GCCAACACCATCAACTGGAAGAAGCTGTTCGGCACCAGCGGCCAGAAA
ACAAAGATCATCAGCAACCGGGGCGAGAACAGCTGCAAGGCTACAGGC
CAAGTGTGCCACGCTCTGTGTAGCCCTGAAGGCTGTTGGGGACCCGAG
CCTAGAGATTGCGTGTCCTGTCGGAATGTGTCCCGGGGCAGAGAATGC
GTGGACAAGTGCAATCTGCTGGAAGGCGAGCCCCGCGAGTTCGTGGAA
AACAGCGAGTGCATCCAGTGTCACCCCGAGTGTCTGCCCCAGGCCATG
AACATTACCTGTACCGGCAGAGGCCCCGACAACTGTATTCAGTGCGCC
CACTACATCGACGGCCCTCACTGCGTGAAAACATGTCCTGCTGGCGTG
ATGGGAGAGAACAACACCCTCGTGTGGAAGTATGCCGACGCCGGACAT
GTGTGCCACCTGTGTCACCCTAATTGCACCTACGGCTGTACAGGCCCT
GGCCTGGAAGGCTGTCCAACAAACGGACCTAAGATCCCCTCTATCGCC
ACCGGCATGGTTGGAGCCCTGCTGCTTCTGCTGGTGGTGGCCCTTGGA
ATCGGCCTGTTCATGTGA
HLA-A2 signal peptide nucleic acid sequence
(SEQ ID NO: 20)
ATGGCTGTGATGGCCCCTAGAACACTGGTGCTGCTGCTGTCTGGTGCC
CTGGCTCTGACTCAGACATGGGCC
huGMCSF Signal Peptide, nucleic acid sequence
(SEQ ID NO: 21)
ATGCTGCTGCTGGTTACATCTCTGCTGCTGTGCGAGCTGCCCCATCCT
GCCTTTCTGCTGATCCCC

### Lentiviral Production and Preparation of Retronectin Plates

VSV pseudotyped lentivirus was produced in 6 well plates. In brief, 293 FT were seeded the night before or the day of at 0.9×10⁶ or 1.4×10⁶ cells/well, respectively. Once the cells had adhered and reached at least 80% confluency a mix of lentiviral plasmid, packaging vector (psPAX2) and VSV-G envelope expressing plasmid (PMD2.G) were transfected using lipofectamine 3000 (Invitrogen®), according to the manufacturer's protocol. After 18 hrs, the media was replaced with 3 mLs of fresh DMEM. Viral supernatants were harvested 48 hrs following changing the media and spun down at 1500 RPM to remove 293 FT cell/debris. Retronectin was coated on 24 well non-tissue culture treated plates at 20 μg/well in PBS−/− for 2 hrs at 37°° C. or overnight at 4° C. Retronectin was removed and washed once with PBS prior to addition of lentiviral supernatant (2 mLs). The plate was then spun at 1500G for 90 minutes at 32° C. to concentrate viral particles onto the retronectin. Lentiviral supernatant was removed immediately prior to transduction of primary T cells or tumor cells. Alternatively, T cells were transduced with polybrene at 8 μg/mL with a spinfection of 800 G for 2 hrs at 32° C.

### T Cell Culture, Transduction, and Isolation

Human PBMCs were activated in T cell growth media with CD3/CD28 microbeads (Invitrogen®) in complete RPMI (100 μl beads/50×10⁶ PBMCs). 48 hrs after activation, activated PBMCs were transferred to Lentiviral-coated Retronectin plates for 48 hrs before being transferred to 6 well plates containing fresh T cell growth media. After an additional 24 hrs in culture, cell transduction was determined by flow cytometry and transduced cells were enriched based on huEGFRt expression. To isolate cells based on EGFR expression, T cells cultures were collected, and activation beads removed. Cells were then stained in 1:100 anti-EGFR-APC antibody in MACS buffer at 4° C. for 30 minutes. Cells were then washed and incubated with anti-APC microbeads (Miltenyi®) for 15-30 minutes at 4° C. Unbound microbeads were then removed by centrifugation and huEGFRt cells were isolated by positive selection on mini-MACS columns. Cells were eluted from the mini-MACS columns and put back into culture in T cell growth media and used within 2 weeks for experiments. To create stable cell lines, cells were collected and transduced as with primary T cells. EGFR selection was performed twice, two weeks apart.

### T Cell Stimulation

In the cases where T cells were stimulated with plate bound antibodies, Maxisorp™ Flat-bottom plates (Invitrogen™) were coated with the indicated amount of anti-human CD3 antibody (HIT3a-Biolegend®) in PBS−/− for 2 hrs at 37° C. Plates were washed twice in basal RPMI before use. For the myeloma cell line stimulation, RPMI18226 and U266 cell lines were collected and resuspended in Cytokine media and aliquoted to U-bottom plates. Similarly, A375 cells were plated 1 day prior to the addition of T cells in DMEM in 96 well flat-bottom plates. The media was exchanged prior to the addition of cognate T cells. Following EGFR+ selection, T cells were collected, counted, and resuspended at the appropriate concentration in Cytokine media and distributed to antibody or APC-bearing wells. For RPMI18226 and U266 experiments, anti-CD3 (HIT3a/Biolegend™) was added at the indicated dose following 1-2 hrs of RPMI18226 and U266/T cell interaction at 37° C. In the case where T cell proliferation was to be tracked, T cells were labelled with Violet Tracking Dye (CTV) according to Biolegend™'s protocol prior to the addition to stimulatory plates. Supernatant was collected at 18-36 hours post stimulation to assess cytokine secretion and proliferation/T cell killing was assessed following 96 hours of stimulation.

### Cytokine Multiplex Assay

Following collection of T cell supernatants cytokines were measured with the Legendplex™ Multi-Analyte Flow Assay Kit for human Th or Th1 cytokines (Biolegend™). The manufacturer's protocol was followed with the following exceptions: 75 μL T cell supernatant was used to measure cytokines and 2 μL of each reagent was used/well. Secreted cytokines were measured by flow cytometry, and the values were normalized to the maximal response of the control group to combine and analyze multiple experiments and normalize for variability between experiments and donors.

### Lentiviral Vector Manufacturing

10 mg of GMP-like transfer plasmid will be produced. The transfer plasmid is to work with third generation packaging system in adherent or suspension HEK293 cells for virus manufacturing. 20-30 liter of GMP lentiviral vector is manufactured at the titer of 10⁷/ml and concentrated to 10⁹/ml.

### Mouse Experiments

NSG Mice (8-10 weeks old) were injected intravenously (tail vein) with 3e6 MM.1S cells stably expressing Gaussia Luciferase-GFP. After 18-20 days, mice were randomized into groups containing roughly equal mean luciferase counts, and T cells were injected by tail vein at the indicated cell counts. Starting at the time of tumor cell injection, once per week, blood samples were collected and Gaussia luciferase levels in the serum were determined by an in vitro luciferase assay. Briefly, 5 μL of clarified plasma was added induplicate to a 96 well plate. Samples were mixed with Gaussia substrate (Coelenterazine, GoldBio #CZ25) according to manufacturer's protocol and measured immediately. For in vivo imaging, animals were imaged following Coelenterazine IP administration. In vitro Luciferase measurements were normalized to measurements taken on day 0 after T cell injection. Animal studies were completed at the Institute of Human Virology at the University of Maryland.

### Example 2: Construction of BCMA-CAR

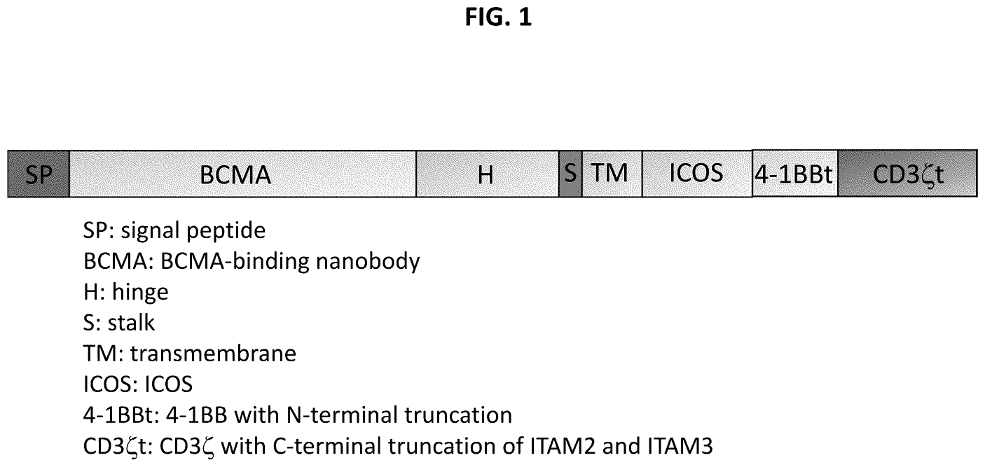

The disclosure herein provides the design of BCMA-CARs comprising a potent BCMA nanobody (Kd=1 nM) driven by the proprietary 3^{rd} generation ICOS and modified 4-1BB co-stimulatory signaling domains and a modified CD3ζ domain (modified 3^{rd} generation BCMA-CAR). FIG. 1 shows a schematic of the structure of an exemplary chimeric antigen receptor comprising BCMA nanobody linked to a chimeric intracellular signaling domain described herein.

### Example 3: Targeted Elimination of BCMA-Positive Multiple Myeloma Cell Lines Using Modified 3^{rd} Generation BCMA-CAR-T Cells Results in Reduced Cytokine Production

Described herein are T cells expressing an exemplary BCMA-CAR of the present disclosure. The disclosure herein provides the design of BCMA CAR molecules and validation of their function on CAR T cell killing of tumor cell lines in vitro.

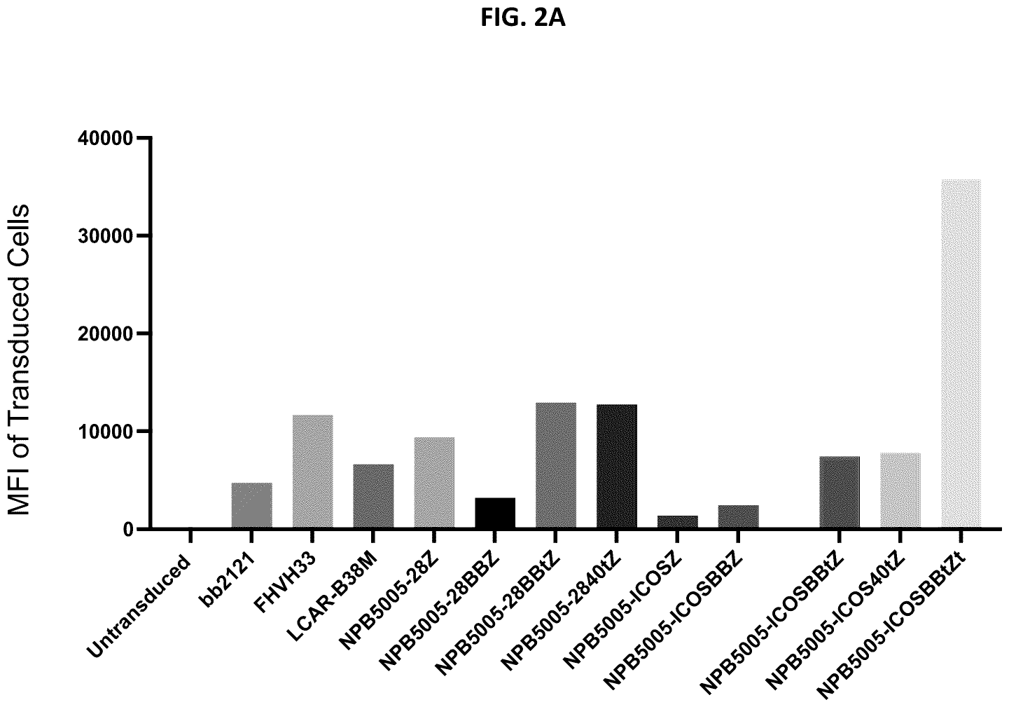

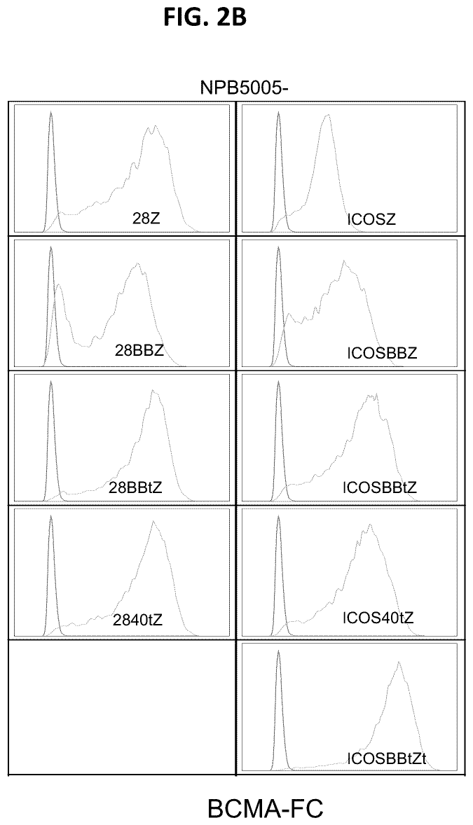

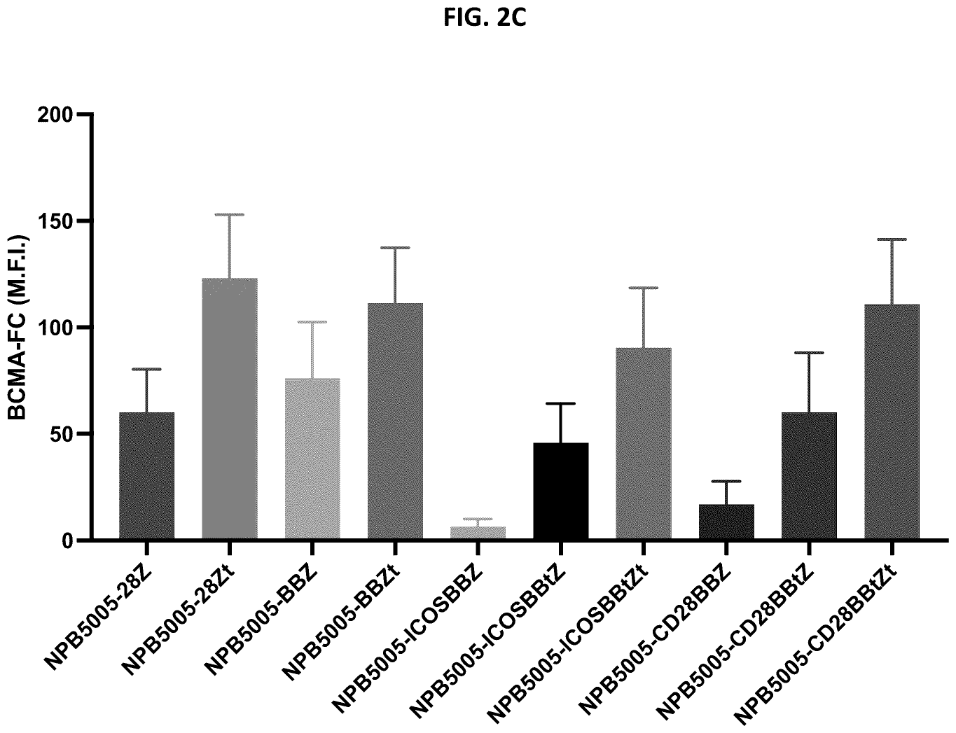

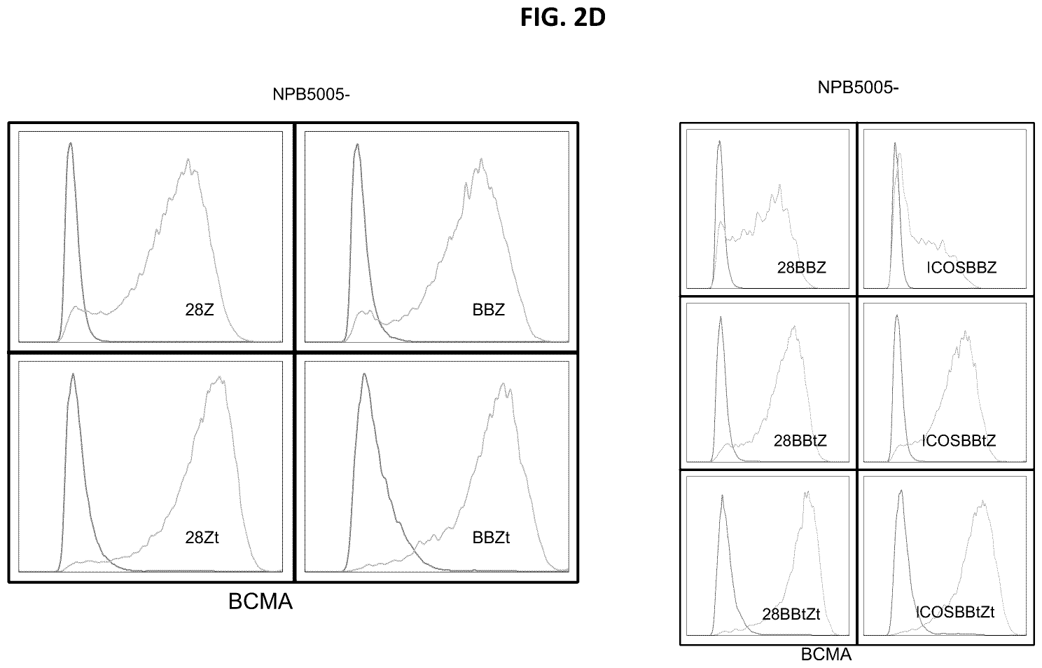

The BCMA-CAR (“Receptor 1” or “NPB5005 BCMA-ICOSBBtZt” or “modified 3rd generation BCMA-CAR”) as described in FIG. 1 was introduced into T cells via transduction. As shown in FIGS. 2A-2C, truncation of the CD3Zeta intracellular signaling domain in a BCMA-CAR of the disclosure increased the surface expression of CAR receptors, improving on surface expression gains made by using modified 3^{rd} generation CAR receptors (FIG. 2A shows bar graphs of expression and FIG. 2B shows corresponding flow plots). FIG. 2C shows BCMA MFI, demonstrating that the surface expression of both second and third generation receptors benefit from truncation of CD3Zeta, and FIG. 2D shows corresponding flow plots.

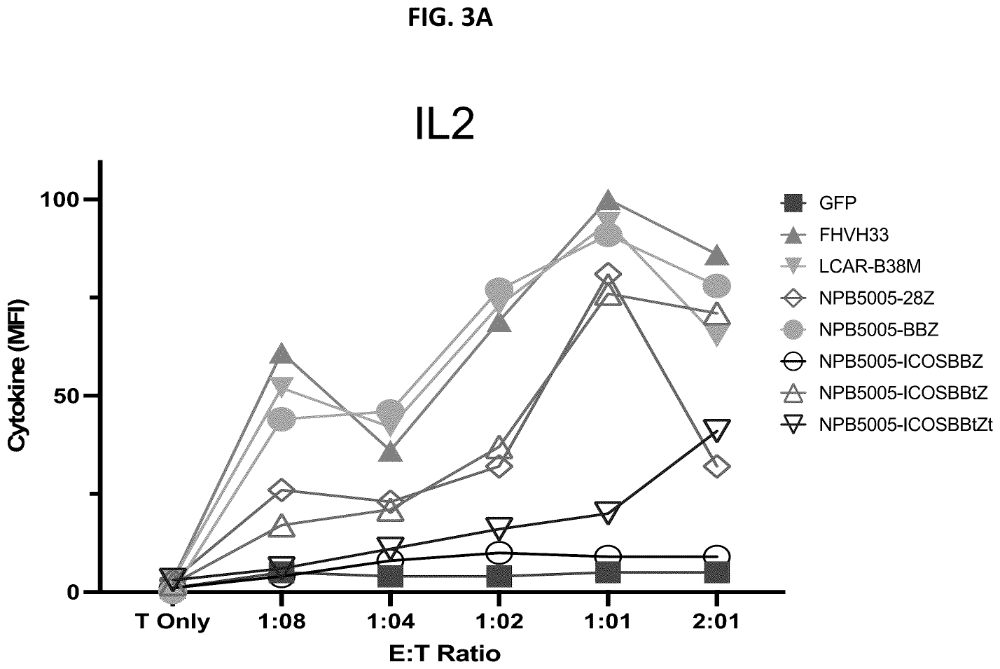

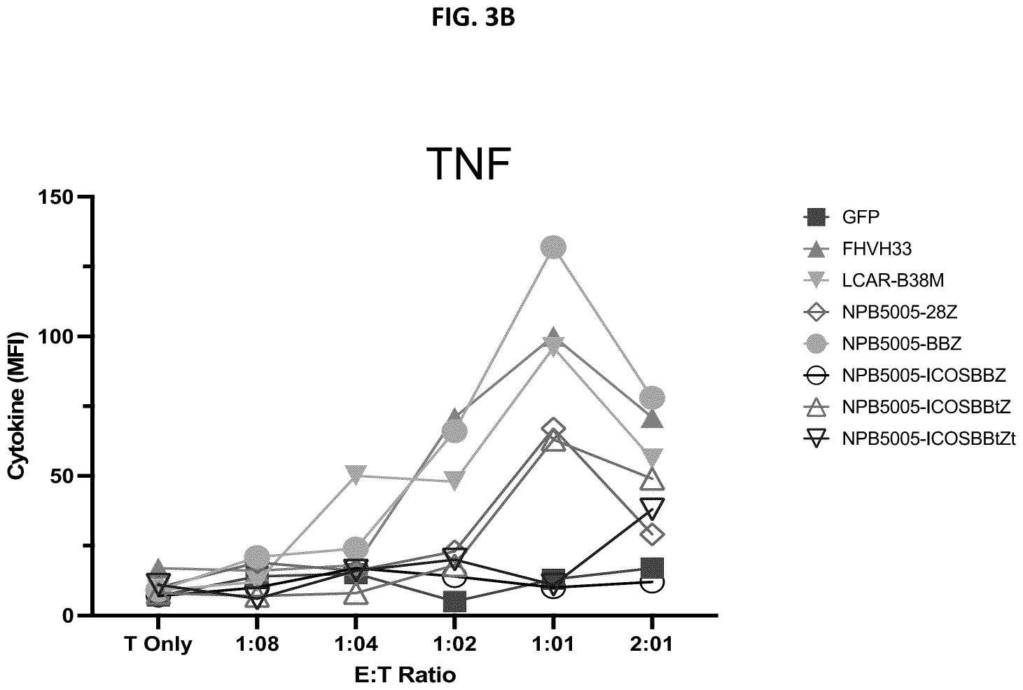

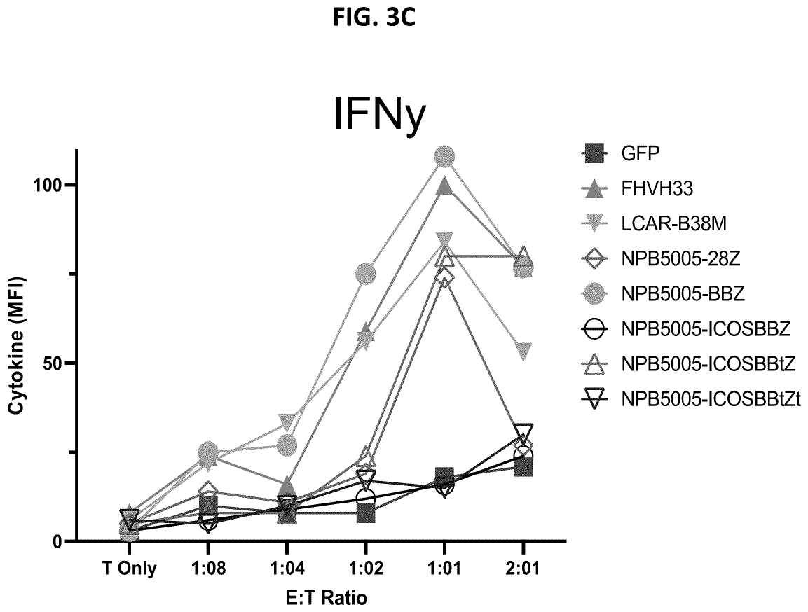

Cytokine production was then assessed. As shown in FIGS. 3A-3C, zeta chain truncation leads to reduced pro-inflammatory cytokine production. IL2 (FIG. 3A), TNFα (FIG. 3B), and IFNγ (FIG. 3C) levels were all reduced, indicating NPB5005-ICOSBBtZt CAR receptor generated less of key inflammatory cytokines relative to the construct with the full-length Zeta intracellular signaling domain and reference constructs.

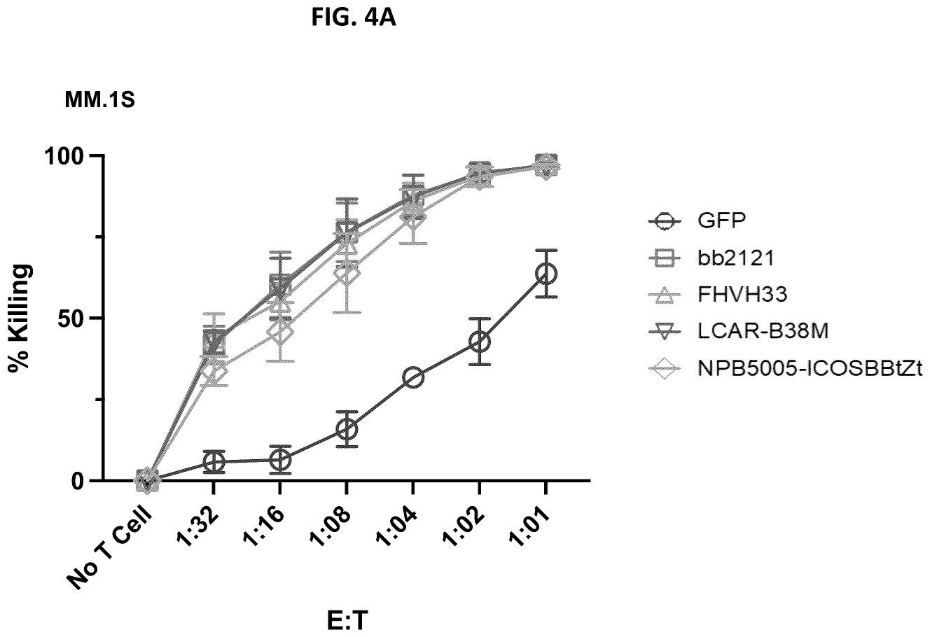

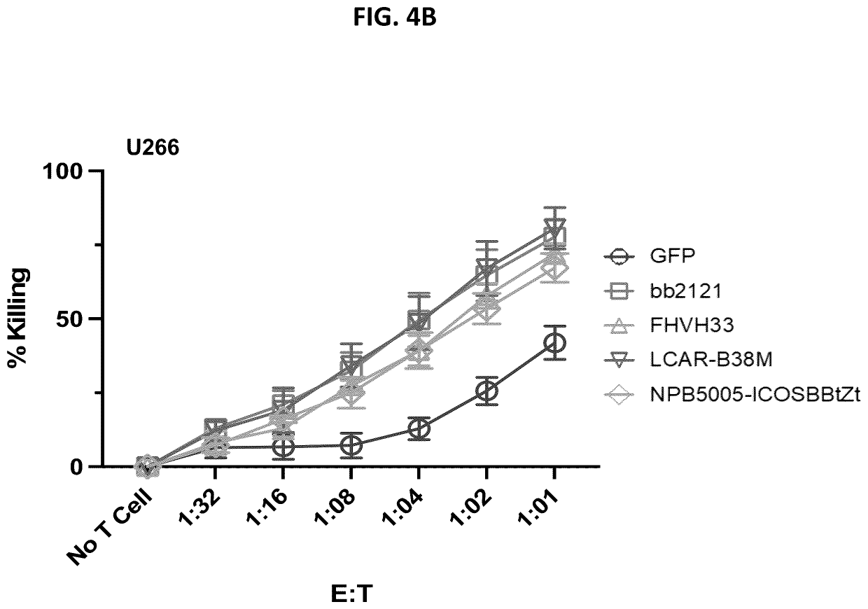

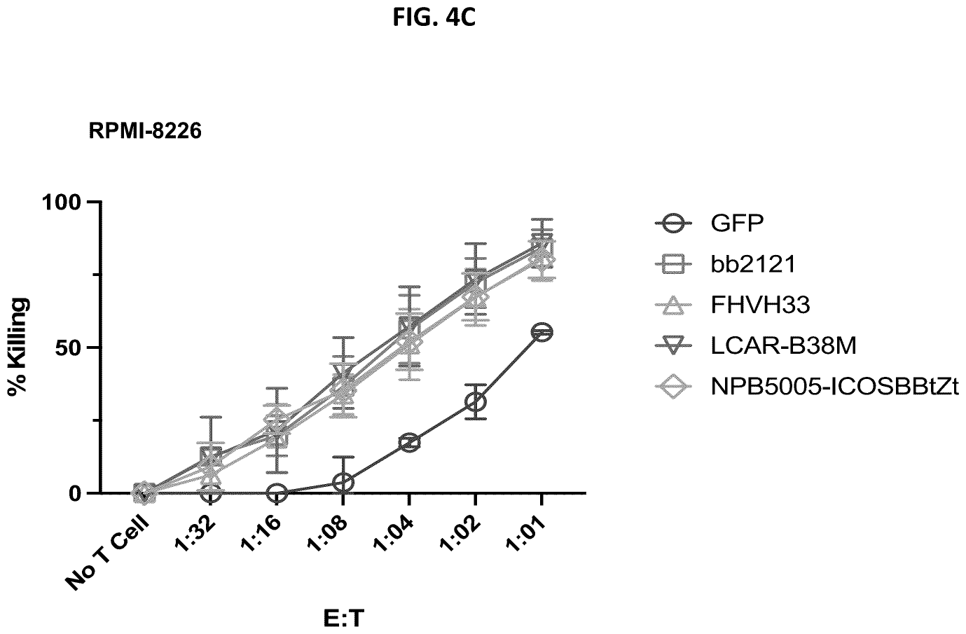

T cells expressing “Receptor 1” were cocultured with multiple myeloma cell lines expressing BCMA, RPMI-8226 and U266, in overnight killing assays to assess the potency of modified 3^{rd} generation BCMA-CAR T cell on target tumor cell killing compared to exemplary clinical BCMA CAR T cell therapies known in the art (bb2121, FHVH33, and LCARB38M) and a GFP control. Target tumor cells included myeloma cell lines expressing BCMA. NPB5005 and control CAR T effector cells (E) were cocultured with target cancer cells (T) at multiple ratios (E:T) from 1:01 to 1:32. The results disclosed herein show that at every ratio tested between 1:01 and 1:32, the modified 3^{rd} generation BCMA-CAR T cells killed a similar percentage of MM.1S, U266, and RPMI-8225 target cells compared to T cells expressing clinical control CARs (FIGS. 4A-4C, respectively).

The exemplary “Receptor 1” or “NPB5005 BCMA-ICOSBBtZt” as described in FIG. 1 was compared with the reference 2^{nd} generation CAR FHVH33 and with the CARs comprised of the same CD8 leader peptide (SEQ ID NO: 6), BCMA-binding nanobody domain (SEQ ID NO: 8), and CD8 hinge (SEQ ID NO: 7) driven by various 2^{nd} generation or 3^{rd} generation signaling domains and a CD3ζ domain (NPB5005-28Z (SEQ ID NO: 12), NPB5005-BBZ (SEQ ID NO: 13), NPB5005-ICOSBBZ (SEQ ID NO: 14), NPB5005-ICOSBBtZ (SEQ ID NO: 15)) in cytokine production (IL-2, IFNγ and TNF). BCMA-CAR T cells were co-cultured with a BCMA positive cell line, RPMI-8226, at various E:T ratios (1:32, 1:16, 1:08, 1:04, 1:02, 1:01 and 2:01). The amount of IL-2, IFNγ and TNFα released when T cells expressing “NPB5005-ICOSBBtZ” were co-cultured with target cells was lower than the cytokine levels released by T cells expressing 2^{nd} generation BCMA-CARs with identical or different BCMA-binding nanobodies (FIGS. 3A-3C). Compared among 3^{rd} generation BCMA-CARs with the same extracellular domain (SEQ ID NO: 1), T cells expressing “NPB5005-ICOSBBtZ” produce similar or slightly higher cytokines as compared to GFP control, and but significantly lower cytokines than T cells expressing than T cells expressing control CARs (FIGS. 3A-3C).

The results of the study described herein show that exemplary BCMA-CAR T cells of the present disclosure eliminate multiple BCMA-positive cancer cells with equivalent potency compared to leading clinical CAR T cells (FIGS. 4A-4c), while producing significantly less cytokine expression (see FIGS. 3A-3C), reducing toxicity and unwanted side effects for the patient.

### Example 4: BCMA-CAR Eliminates BCMA-Positive Myeloma Cell Lines With Superior Efficacy During Long-Term Repetitive Co-Culture

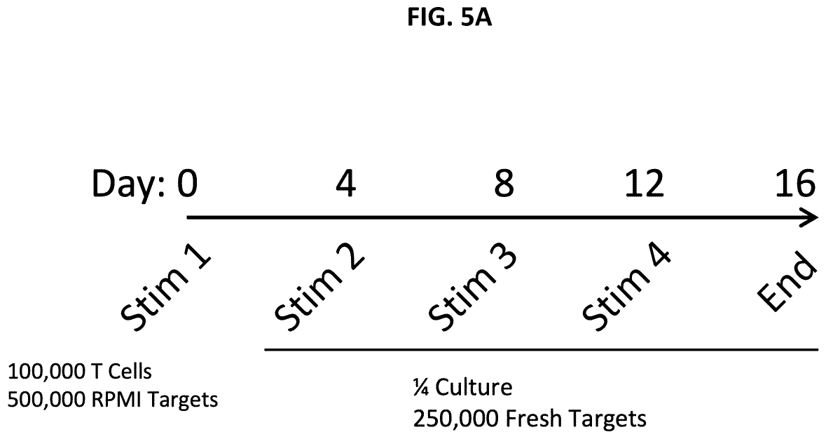

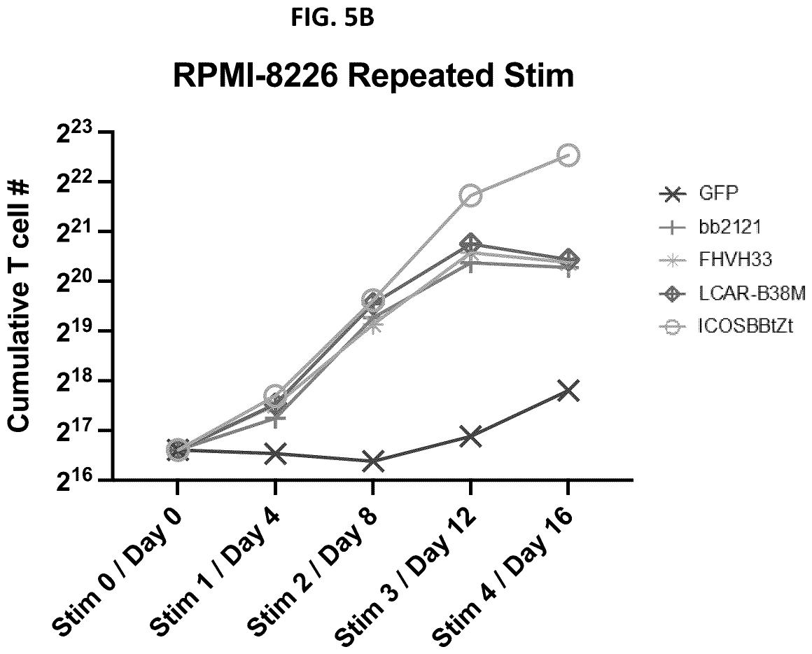

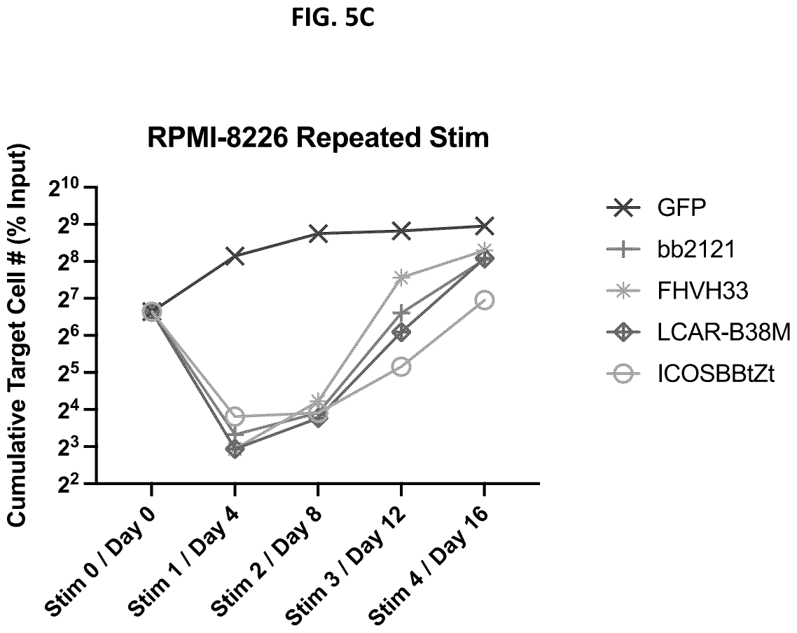

In vitro studies described herein show increased T cell proliferation and persistence by T cells transduced with the modified 3^{rd} generation BCMA-CARs (“Receptor 1” or “NPB5005-ICOSBBtZt”) compared to T cells with control clinical CARs (when co-cultured with a BCMA-expressing myeloma cell line, RPMI-8226, based on an exemplary long term repetitive co-culture assay (FIG. 5A). 100,000 CAR T cells were cultured with 500,000 RPMI-8266 cell targets. Cultures were stimulated with 250,000 additional fresh RPMI-8226 target cells every 4 days (on Day 4, Day 8, and Day 12) and cultured for a total of 16 days. FIGS. 5B and 5C show T cell number and target cell number, respectively, at the end of each stimulation, demonstrating enhanced long-term functional cytolytic potential of the NPB5005-ICOSBBtZt CAR construct relative to reference CARs.

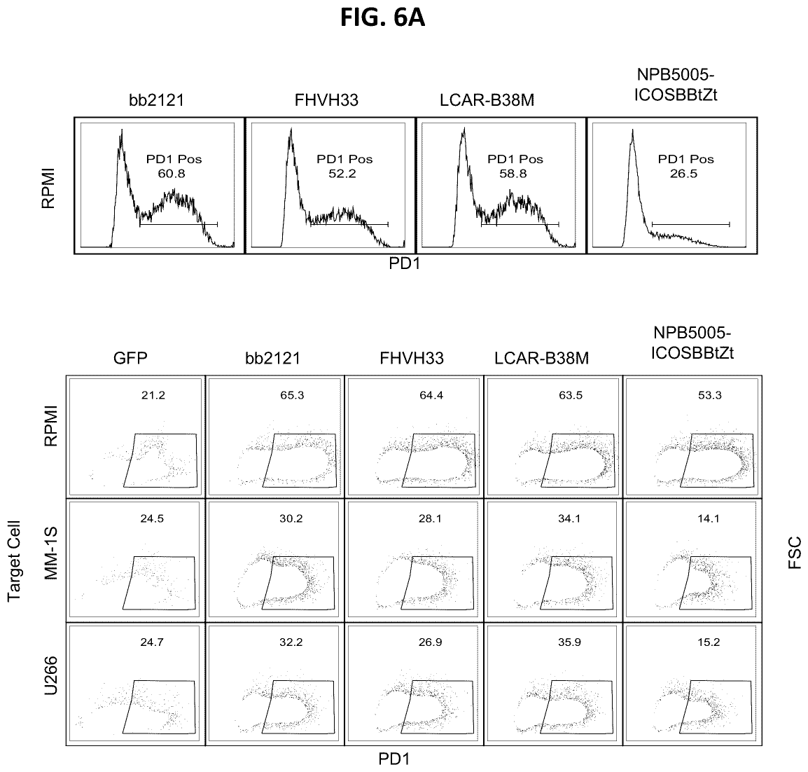

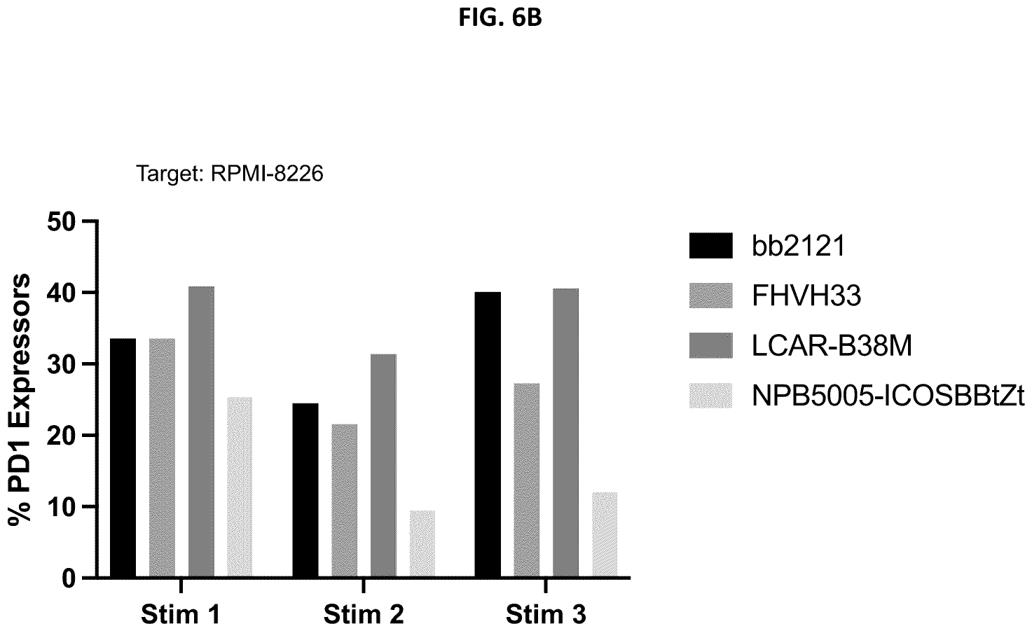

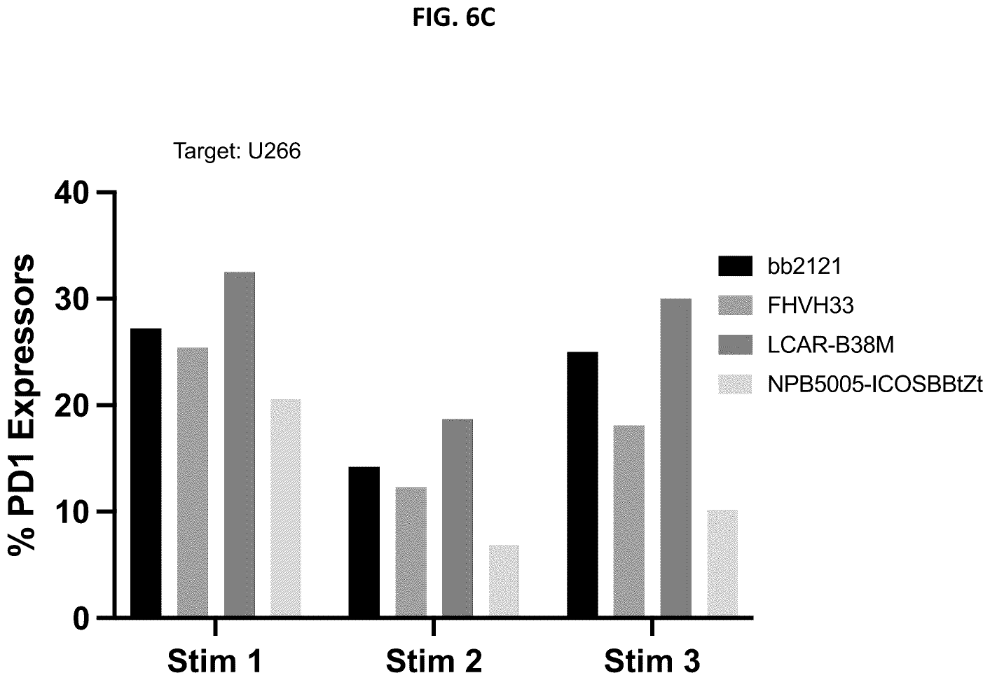

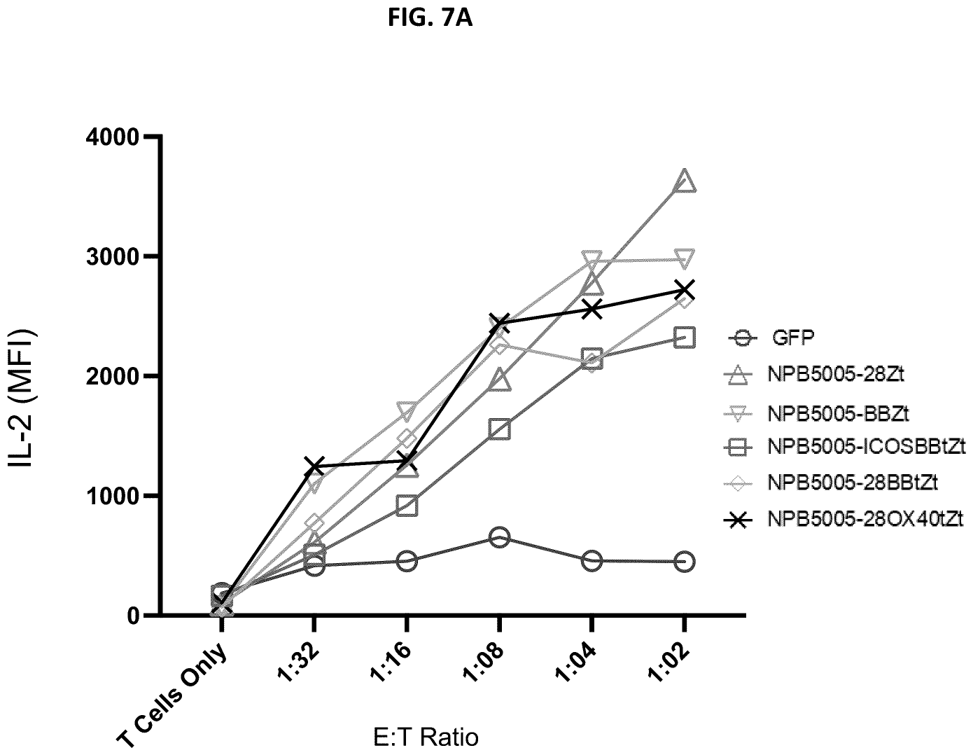

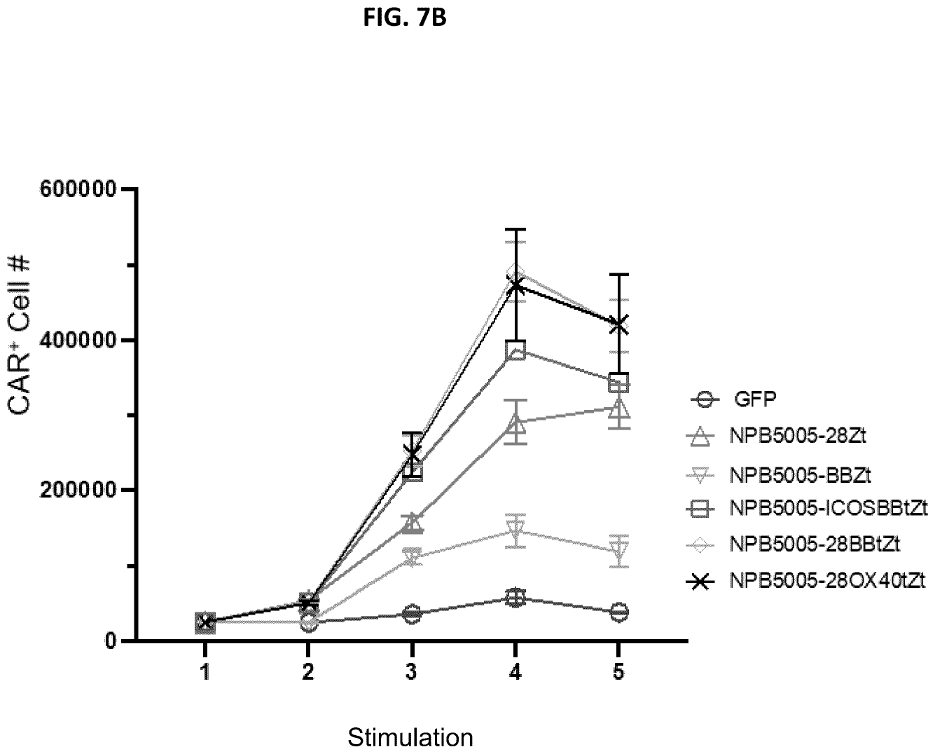

NPB5005-ICOSBBtZt resulted in lower PD-1 expression in antigen-stimulated CAR-T, as indicated in FIGS. 6A-6C. The decreased PD-1 expression on the surface of T cells was observed in both RPMI-8226 (FIG. 6B) and U266 (FIG. 6C) cell lines. It was further observed that the 3^{rd} generation costimulatory domains are necessary for the full functionality of truncated CD3zeta chain-based CAR receptors of the present disclosure, as demonstrated by IL2 production comparison in FIG. 7A and CAR positive cells in FIG. 7B.

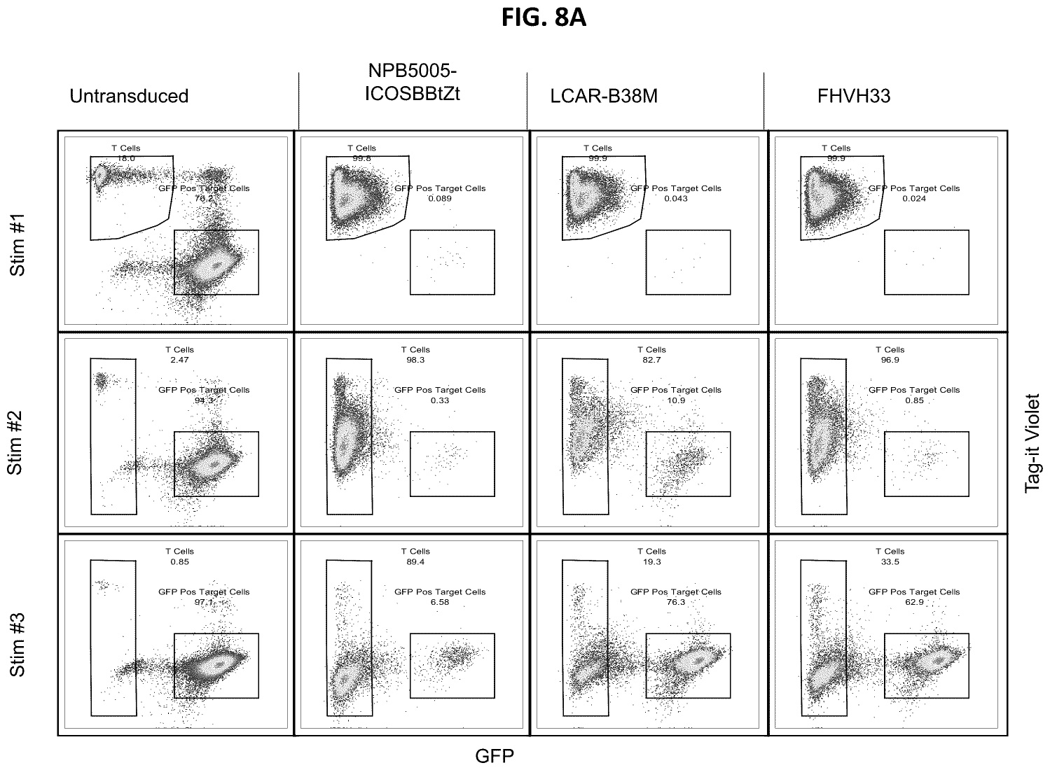

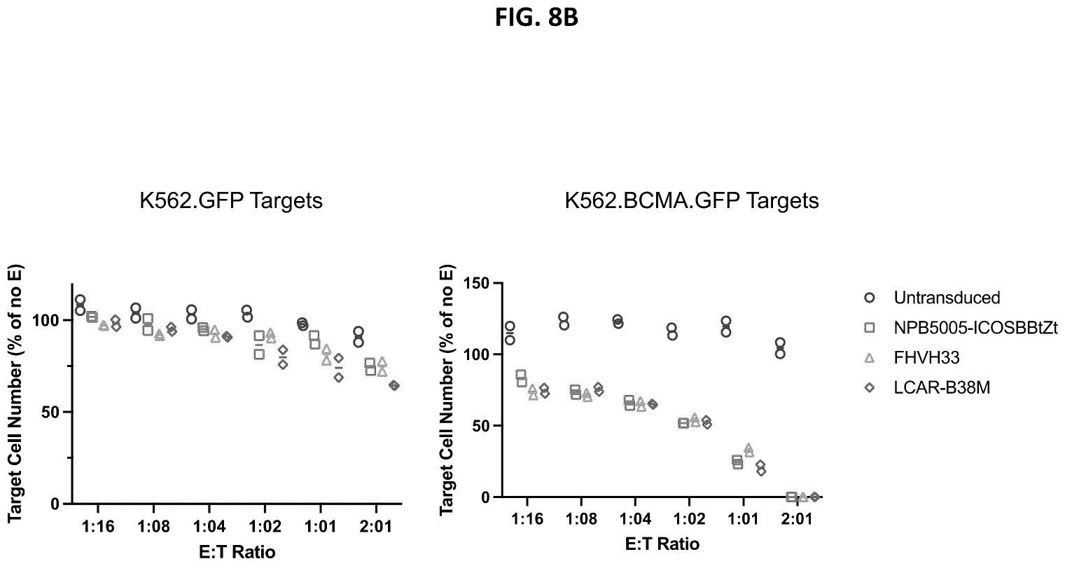

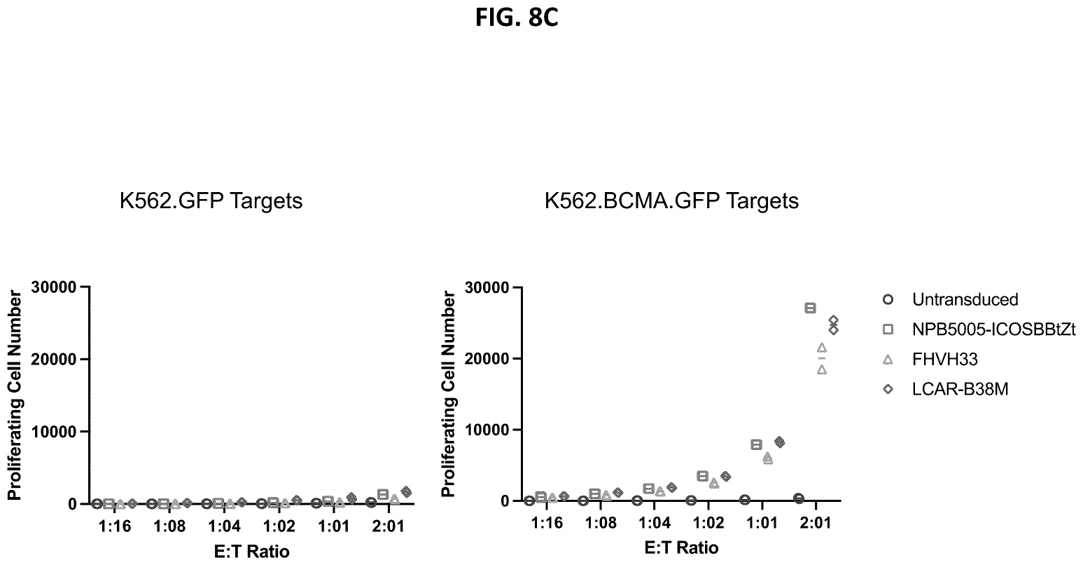

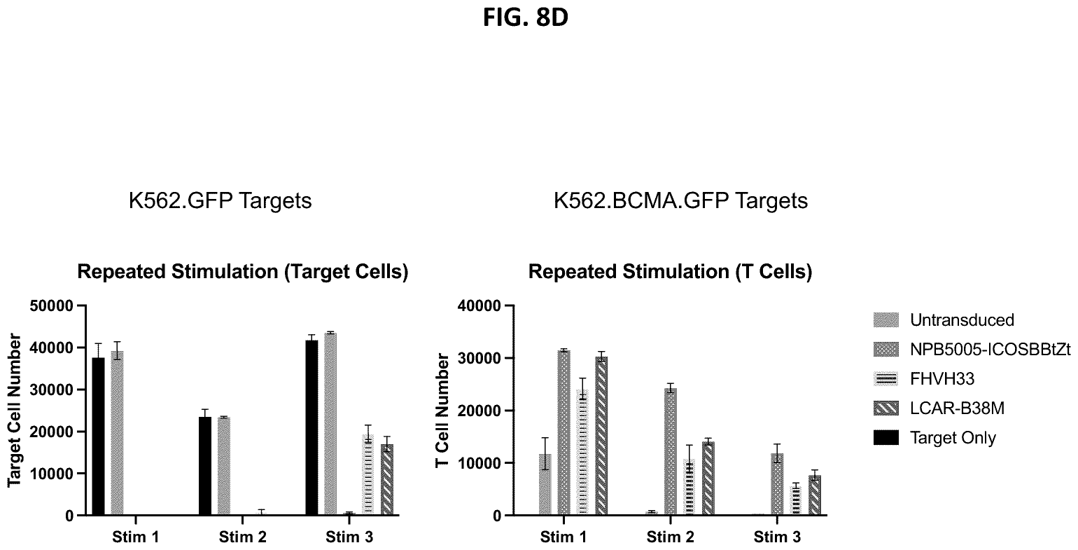

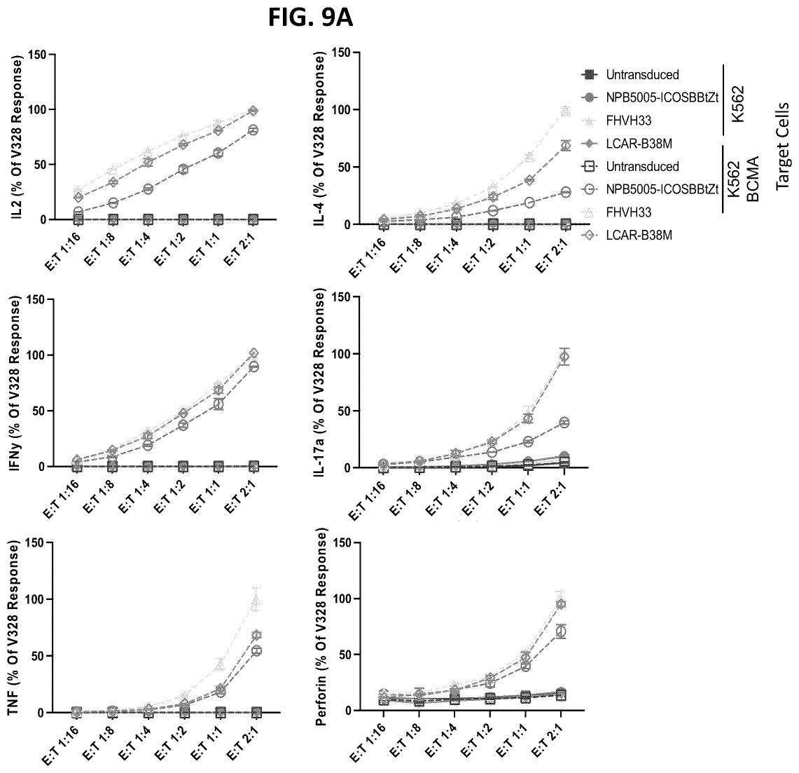

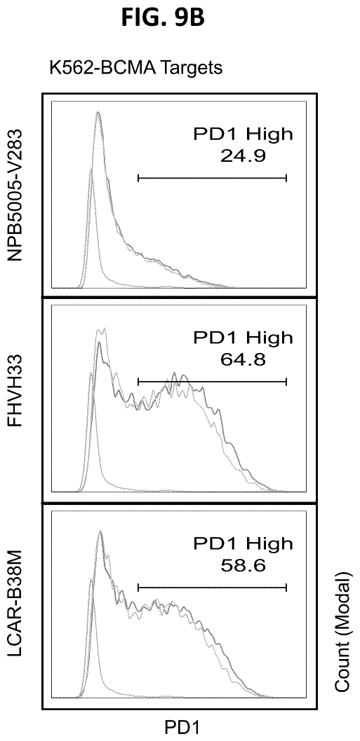

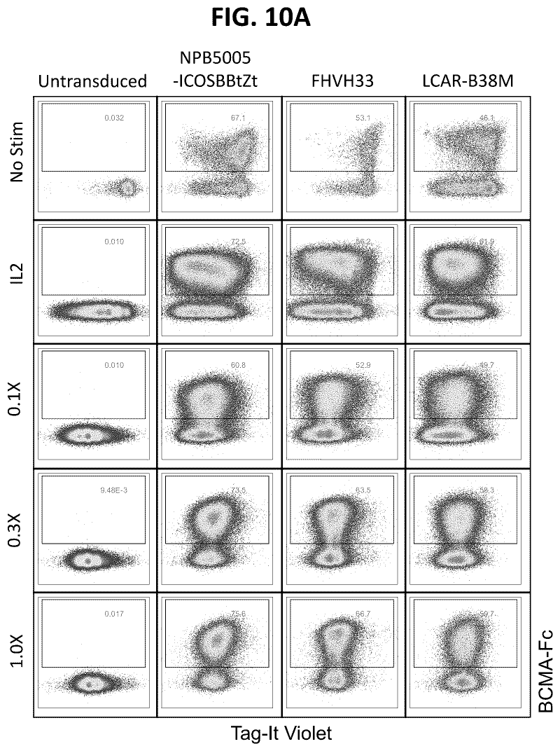

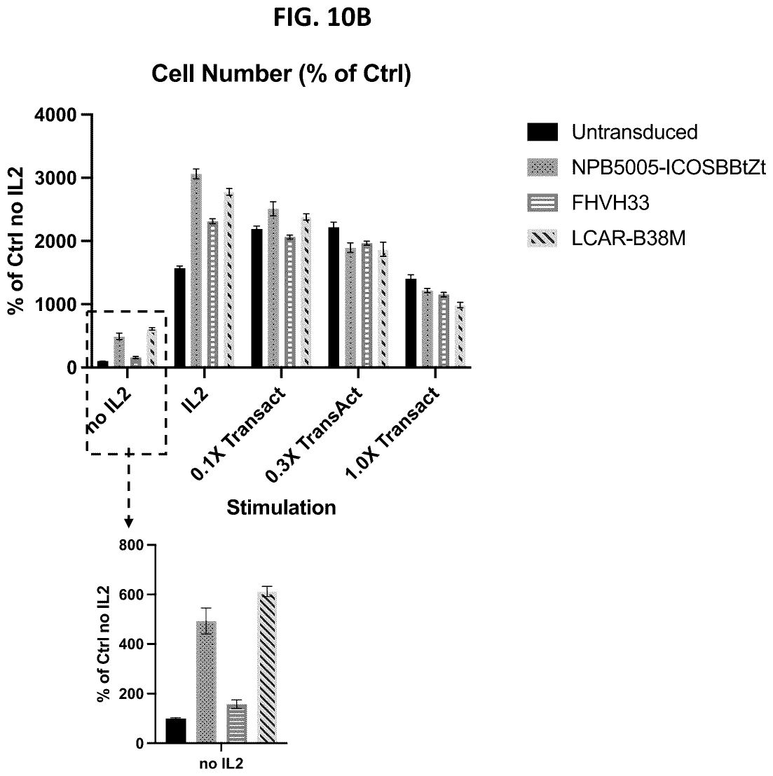

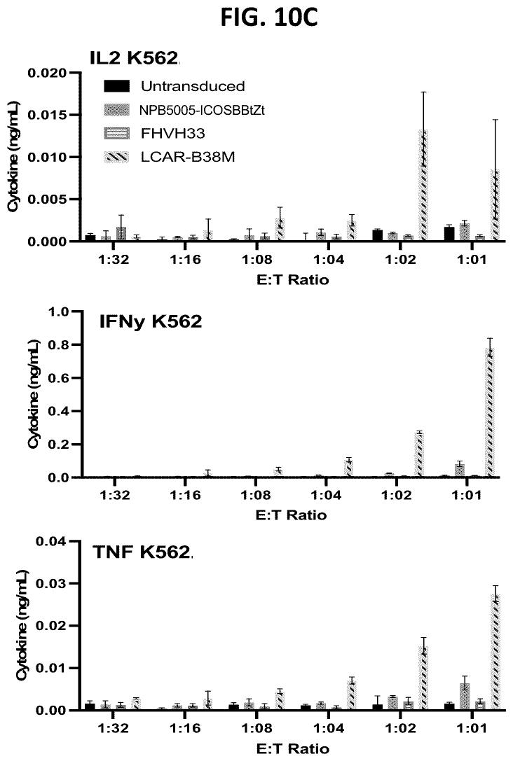

BCMA-CAR of the present disclosure was cloned into clinical vector format, which lacks a eukaryotic selection marker. Functionality of the BCMA-CAR was assessed from the new vector, including proliferative and cytolytic phenotypes (FIGS. 8A-8D) and reduced cytokine production and PD1 expression (FIGS. 9A-9B). Intermediate CAR-intrinsic tonic signaling (FIGS. 10A-10C) was also determined, with NPB5005-ICOSBBtZt demonstrating a level of tonic signaling in-between the two reference CAR-T cells; cell survival was comparable to LCAR-B38M in the absence of IL2 or CAR ligand (FIG. 10A-10B), but tonic cytokine production was strikingly reduced (FIG. 10C).

### Example 5: BCMA-CAR Shows Dose-Dependent Efficacy in a Xenograft Murine Model in Vivo

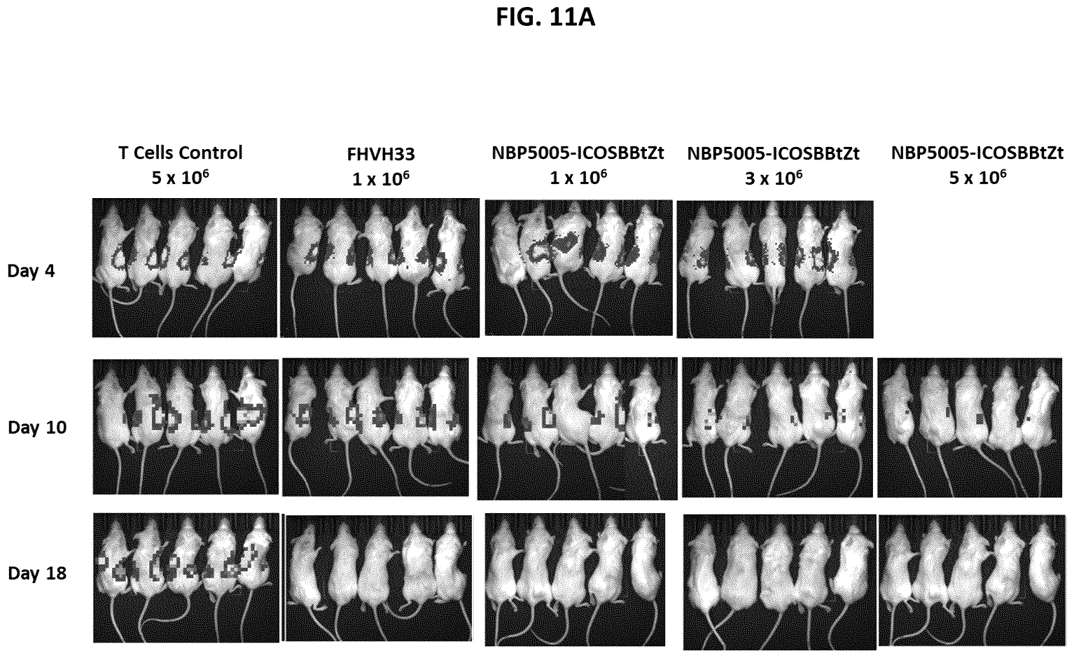

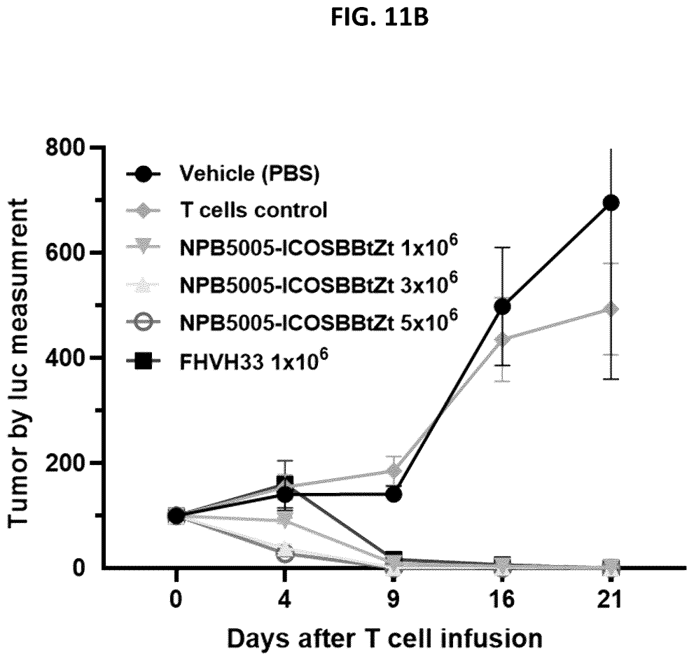

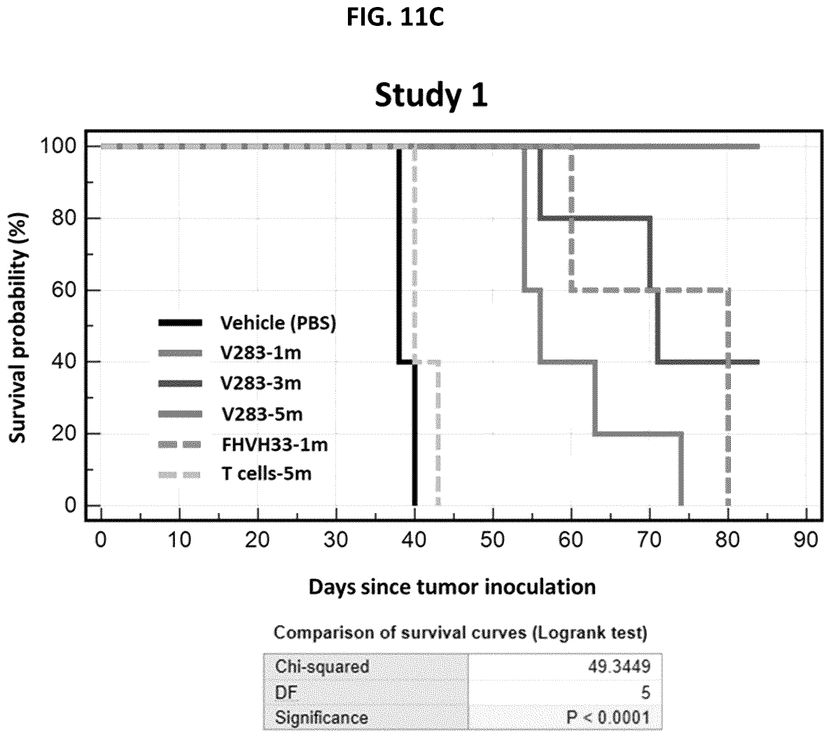

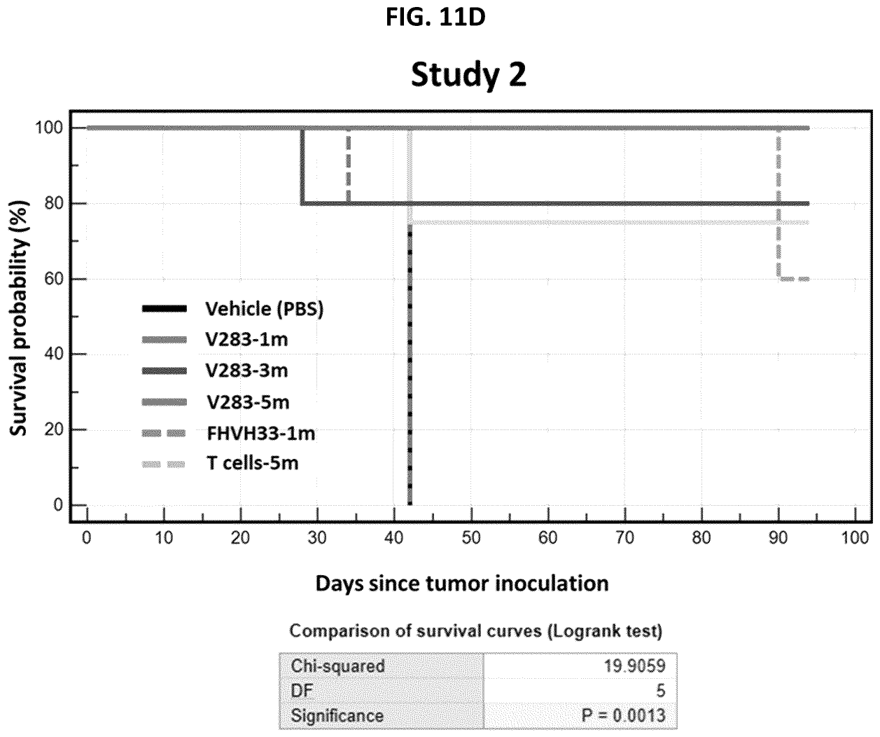

To assess efficacy in vivo, MM.1S cells were injected into mice at 1×10⁶, 3×10⁶, and 5×10⁶ via tail vein injection. Intravital imaging of tumor burden at days 4, 10, and 18 (FIG. 11A), as well as quantification of secreted Gaussia luciferase (FIG. 11B), demonstrated significantly reduced tumor burden in all doses tested, and a dose-dependent effect. Survival curves of female (FIG. 11C) and male (FIG. 11D) mice demonstrated enhanced survival of treated subjects, with mice receiving NPB5005-ICOSBBtZt demonstrating about 100% survival over 90-days post-dose.

Taken together, the results described herein show that T cells expressing the modified 3^{rd} generation BCMA-CAR of the present disclosure (e.g., NPB5005-ICOSBBtZt) can be used to treat cancer with high efficacy and significantly lower adverse events. Specifically, expression of NPB5005-ICOSBBtZt results in increased effector function, proliferation, and persistence, while minimizing cytokine release. As shown above, NPB5005-ICOSBBtZt kills multiple myeloma cell lines expressing BCMA with a comparable efficacy as the leading BMCA-CAR T cell therapies, producing reduced toxic cytokines and expressing lower levels of surface PD-1. Therefore, NPB5005-ICOSBBtZt is more resistant to negative signaling from cells in the tumor microenvironment. Following repeated stimulation with target cells, NPB5005-ICOSBBtZt proliferates to higher numbers compared to alternative BCMA-CAR T cells.

What is claimed is:

1. A chimeric antigen receptor that binds B cell maturation antigen (BCMA-CAR), comprising:
(a) an extracellular domain comprising the amino acid sequence of SEQ ID NO: 8;
(b) a transmembrane domain; and
(c) a chimeric intracellular domain comprising a first, a second and at least a third signal transduction domain,
wherein the first signal transduction domain comprises an ICOS intracellular domain according to SEQ ID NO: 2, the second signal transduction domain comprises a truncated CD137 (4-1BB) intracellular domain according to SEQ ID NO: 3, and the at least third signal transduction domain comprises a truncated CD3ζ domain according to SEQ ID NO: 4.

2. The BCMA-CAR of claim 1, wherein the extracellular domain further comprises a signal peptide, a hinge, an ICOS extracellular stalk, or a combination thereof.

3. The BCMA-CAR of claim 1, wherein the extracellular domain further comprises a CD8a signal peptide, a CD8a hinge, an ICOS extracellular stalk, or a combination thereof.

4. The BCMA-CAR of claim 1, wherein the extracellular domain further comprises a CD8a signal peptide according to SEQ ID NO: 6, a CD8a hinge according to SEQ ID NO: 7, an ICOS extracellular stalk according to SEQ ID NO: 9, or a combination thereof.

5. The BCMA-CAR of claim 1, wherein:
a) the extracellular domain comprises the amino acid sequence of SEQ ID NO: 1; and/or
b) the transmembrane domain comprises the amino acid sequence of SEQ ID NO: 10.

6. The BCMA-CAR of claim 1, wherein the BCMA-CAR comprises the amino acid sequence of SEQ ID NO: 5.

7. A chimeric antigen receptor that binds B cell maturation antigen (BCMA-CAR), comprising:
(a) an extracellular domain comprising the amino acid sequence of SEQ ID NO: 1;
(b) a transmembrane domain comprising the amino acid sequence of SEQ ID NO: 10; and
(c) a chimeric intracellular domain comprising a first, a second and at least a third signal transduction domain,
wherein the first signal transduction domain comprises an ICOS intracellular domain according to SEQ ID NO: 2, the second signal transduction domain comprises a truncated CD137 (4-1BB) intracellular domain according to SEQ ID NO: 3, and the at least third signal transduction domain comprises a truncated CD3ζ domain according to SEQ ID NO: 4.

8. A chimeric antigen receptor that binds B cell maturation antigen (BCMA-CAR) comprising the amino acid sequence of SEQ ID NO:5.

9. A nucleic acid encoding the BCMA-CAR of claim 1, or a vector comprising said nucleic acid.

10. A cell comprising the nucleic acid or vector of claim 9.

11. The cell of claim 10, wherein the cell is:
a) a modified T cell; or
b) a modified NK-T cell.

12. The cell of claim 10, wherein the cell is:
a) an allogeneic T cell; or
b) an autologous T cell.

13. The cell of claim 11, wherein the modified T cell is a naïve T cell, an early memory T cell, a stem cell-like T cell, a stem memory T cell (TSCM), a central memory T cell (TCM), or a regulatory T cell (Treg).

14. The cell of claim 10, wherein the cell co-expresses the BCMA-CAR with at least one endogenous co-stimulatory molecule selected from CD28, CD2, OX-40, ICOS, CD28, CD3, CD4, CD8, CD40L, and a combination thereof.

15. A modified T cell comprising:
(a) a modification of an endogenous sequence encoding a T cell Receptor (TCR), wherein the modification reduces or eliminates a level of expression or activity of the TCR; and
(b) the BCMA-CAR according to claim 1.

16. The modified T cell of claim 15, further comprising:
a) a modification of an endogenous sequence encoding a component of major histocompatibility complex (MHC) class I (MHC-I), wherein the modification reduces or eliminates a level of expression or activity of the MHC-I; and/or
b) at least one endogenous co-stimulatory molecules selected from CD28, CD2, OX-40, ICOS, CD28, CD3, CD4, CD8, CD40L, and a combination thereof.

17. A pharmaceutical composition comprising the cell of claim 10.

18. A method of producing a plurality of modified T cells, wherein the method comprises:
a) providing a plurality of primary T cells;
b) providing a composition comprising the nucleic acid or the vector of claim 9; and
c) introducing into the plurality of primary T cells of (a) the composition of (b), to produce a plurality of modified T cells under conditions that stably express the BCMA-CAR within the plurality of modified T cells.

19. A method of treating a disease or disorder comprising administering to a subject in need thereof a therapeutically effective number of the cell of claim 10.

20. The method of claim 19, wherein the disease or disorder is a cancer, an autoimmune disease or disorder, or an inflammatory disease,
wherein the cancer is selected from acute leukemia, acute lymphoblastic leukemia (ALL), acute lymphocytic leukemia, B cell, T cell or FAB ALL, acute myeloid leukemia (AML), acute myelogenous leukemia, chronic myelocytic leukemia (CML), chronic lymphocytic leukemia (CLL), hairy cell leukemia, myelodysplastic syndrome (MDS), Hodgkin's lymphoma, Hodgkin's disease, non-Hodgkin's lymphoma, and multiple myeloma
